(19) United States
(12) Patent Application Publication
(10) Pub. No.: US 2022/0370493 A1
(43) Pub. Date: Nov. 24, 2022

(54) METHODS AND COMPOSITIONS FOR GENERATING NITRIC OXIDE AND USES THEREOF

(71) Applicant: THIRTY HOLDINGS LIMITED, London (GB)

(72) Inventors: Hugh Semple MUNRO, London (GB); Christopher Barry WOOD, London (GB)

(21) Appl. No.: 17/616,468

(22) PCT Filed: Jun. 2, 2020
(86) PCT No.: PCT/GB2020/051328

§ 371 Date: Dec. 3, 2021

(30) Foreign Application Priority Data
Jun. 4, 2019 (GB) 1907969.8
Oct. 22, 2019 (GB) 1915280.0
Apr. 23, 2020 (GB) 2005980.4

(51) Int. Cl. A61K 33/00; A61K 31/194; A61K 31/047; A61K 9/00

(52) U.S. Cl. CPC A61K 33/00; A61K 31/194; A61K 31/047; A61K 9/008

(57) ABSTRACT

The invention provides a combination, kit or composition comprising: (i) one or more nitrite salt; (ii) a proton source comprising one or more acid selected from organic carboxylic acids and organic non-carboxylic reducing acids; and (iii) one or more organic polyol. On reaction of the one or more nitrite salt with the proton source in the presence of the one or more organic polyol, the combination, kit or composition provides reaction products which include nitric oxide, optionally other oxides of nitrogen and/or optionally precursors thereof and which are useful, for example, in the treatment of various disorders.

### FIELD OF THE INVENTION

The present invention relates to methods and compositions for generating nitric oxide, optionally other oxides of nitrogen and/or optionally precursors thereof, and uses thereof such as for delivering nitric oxide, optionally other oxides of nitrogen and/or optionally precursors thereof to organisms and microorganisms, for example for treating disorders responsive to nitric oxide.

### BACKGROUND OF THE INVENTION

Nitric oxide (NO) and nitric oxide precursors have been extensively studied as potential pharmaceutical agents. Nitric oxide is a potent vasodilator which is synthesised and released by vascular endothelial cells and plays an important role in regulating, inter alia, vascular local resistance and blood flow. In mammalian cells, nitric oxide is principally produced along with L-citrulline by the enzymatic oxidation of L-arginine. Nitric oxide is also released from the skin by a mechanism which appears to be independent of NO synthase enzyme. Nitric oxide is also involved in the inhibition of both platelet and leucocyte aggregation and adhesion, the inhibition of cell proliferation, the scavenging of superoxide radicals and the modulation of endothelial layer permeability. The role of nitric oxide in cancer treatment was discussed in Biochemistry (Moscow), 63(7), 802-809 (1998), the disclosure of which is incorporated herein by reference. Nitric oxide has been shown to possess antimicrobial properties, as reviewed by F C Fang in J. Clin. Invest. 99(12), 2818-2825 (1997) and as described for example in WO 95/22335 and WO 02/20026 (Aberdeen University), the disclosures of which are incorporated herein by reference. Other known uses and applications of systems for generation of nitric oxide, other oxides of nitrogen and precursors thereof are given below in the description of the present invention.

There remain substantial problems in connection with the efficient generation and delivery of nitric oxide, other oxides of nitrogen and precursors thereof to organisms and cells for treatment. A widely adopted system for the generation of nitric oxide relies on the acidification of nitrite salts using a mineral acid to produce initially nitrous acid (HNO₂) in equimolar amounts in comparison with the starting nitrite, which nitrous acid then readily decomposes to nitric oxide and nitrate with hydrogen ions and water. The decomposition can be represented by the following balanced equation (1):

HNO₂→2NO+NO₃⁻+H^{+H}₂O (1)

It has been conventional to perform the acidification of the nitrite at a pH of less than about 4, at which the formation of nitrous acid is generally favoured, in order to seek to maximise the yield of NO. However, the use of pH<4 is not suitable for in vivo use where the acid is in contact with animal tissue. A higher pH would be more benign to cells and living systems, but at pH greater than 4 the prior systems have not produced satisfactory yields of NO. To seek to increase the amount of NO generated above pH 4 large quantities of nitrite are required, which is impractical in therapeutic applications and uneconomic. In addition, the conversion represented by Equation (1) is not readily controllable in view of the short half-life of nitrous acid, so that controlled release of nitric oxide for therapeutic use is difficult. The reaction between one or more nitrite salt and a proton source to generate nitric oxide, optionally other oxides of nitrogen and/or optionally precursors thereof is referred to herein as the “NOx generating reaction” or the “reaction to generate NOx” or like wording, and “NOx” is used to refer to the products of the acidification of nitrite, particularly nitric oxide, other oxides of nitrogen and precursors thereof both individually and collectively in any combination. It will be understood that each component of the generated NOx can be evolved as a gas, or can pass into solution in the reaction mixture, or can initially pass into solution and subsequently be evolved as a gas, or any combination thereof.

WO 00/53193, the disclosure of which is incorporated herein by reference, describes a cream or ointment for treating skin ischaemia and to promote wound healing, in which the proton source is ascorbic acid. Example 1 describes a gel based on KY Jelly™, and in Example 7 the gel was tested both in direct contact with skin and where the skin was protected by a membrane. It was claimed that the use of ascorbic acid avoids significant skin inflammation (WO 00/53193, page 2). In practice, however, the extent of skin inflammation due to the low pH of the gel was unsatisfactory when the gel contacted the skin directly, and the skin-protective membrane attenuated the effect of the gel when the membrane was present. The result is that the gel has not been marketed. The compositions of WO 00/53193 are polyol free.

WO 02/20026, the disclosure of which is incorporated herein by reference, describes a skin preparation for treating a drug resistant infection of the skin, in which the proton source is citric acid or salicylic acid. A nitrite containing composition and an acid containing composition are dispensed from a twin barreled dispenser, which compositions are then mixed to cause the acid to react with the nitrite before being spread on the skin. Propylene glycol and polyethylene glycol are taught to be optional preservatives and glycerin (glycerol) is taught to be an optional thixotropic agent for use with the nitrite composition. Propylene glycol was used in a pair of creams of respectively citric acid and nitrite salt, which were to be mixed in situ to initiate the reaction between the acid and the nitrite salt (e.g. WO 02/20026, Example 3, Formulation 1). Glycerol was used with cetostearyl alcohol in a pair of lotions of respectively citric acid and nitrite salt, which were to be mixed in situ to initiate the reaction between the acid and the nitrite salt (e.g. WO 02/20026, Example 3, Formulation 3). The preferred pH of the reaction mixture is a pH of 5 or below, especially 4 or below, which will be expected to cause undesirable skin inflammation. Nasal sprays are also taught, which may use reducing acids such as ascorbate or ascorbic palmitate so that higher pH's can be used to avoid irritating the sensitive nasal mucosae. However, it is acknowledged (WO 02/20026, page 16, second paragraph) that the higher pH will slow the reaction.

U.S. Pat. No. 6,103,275 (published 15 Aug. 2000), the disclosure of which is incorporated herein by reference, describes the use of a reductant such as ascorbic acid with an organic acid having pKa between 1 and 4, such as maleic acid, to acidify the nitrite salt. A viscous (gel) composition is used to slow down the release of the reaction products for topical use. The acid and the nitrite salt are kept separate until the generation of the nitric oxide is to start, and the reductant is stated to be included in at least one of the first and second gels. The pH range at which the method should be used is not specified. However, the fact that the buffer components are referred to as acids may indicate that these compounds are predominantly present in the protonated form, therefore the pH of the composition should be substantially lower than 4. The presence of acids with pKa between 1 and 4 ensures good buffering capacity of the formulation at that pH. Whilst incorporation of such acids is a convenient way of ensuring that pH is maintained at a level such that a continuous efficiency of converting nitrite to nitric oxide is maintained, the low pH will be expected to cause substantial undesirable skin irritation on contact with the skin. The compositions of U.S. Pat. No. 6,103,275 are polyol free.

In WO 2003/013489, the disclosure of which is incorporated herein by reference, 3% polyvinyl alcohol (PA) was proposed as gel base for respectively citric acid and a nitrite salt, which were to be mixed together in situ (WO 2003/013489, Example 7). However, the test data (WO 2003/013489, Tables 11 and 12) show that stable gels could not be formed with PA, and PA compositions were never mixed or used together. Apart from the above proposal, which was not followed through to a final composition, the compositions of WO 2003/013489 are polyol free.

US Patent Application No. 2005/0037093, the disclosure of which is incorporated herein by reference, describes nitric oxide generating compositions based on the nitrite-acid reaction and mentions optional excipients including polyvinyl alcohol, propylene glycol and polyethylene glycol.

Chinese Patent Application No. CN 101028229, the disclosure of which is incorporated herein by reference, describes cosmetic products which generate nitric oxide by the reaction of a nitrite with an acid. The optional use of inter alia glycerin, propylene glycol and glycerin monostearate as additional ingredients is taught. Trihydroxyethylamine is further mentioned as an ingredient in a specific Example.

Chinese Patent Application No. CN 101062050, the disclosure of which is incorporated herein by reference, describes hair growth promoting products which generate nitric oxide by the reaction of a nitrite with an acid. The optional use of inter alia glycerin, propylene glycol and glycerin monostearate as additional ingredients is taught. D-pantothenyl alcohol and a combination of panthenol and inositol are mentioned as ingredients in specific Examples.

WO 2008/110872, the disclosure of which is incorporated herein by reference, describes foamable nitric oxide donor compositions which optionally contain a polar solvent, for example selected from a polyol and a polyethylene glycol (paragraphs [0055] and [0056]). Specific polyols are stated to be propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol, dibutylene glycol, glycerin, butane-1,2,3-triol, butane-1,2,4-triol and hexane-1,2,6-triol. Polyvinyl alcohol, polyethylene glycol 1000 (PEG 1000), PEG 4000, PEG 6000 and PEG 8000 are mentioned in a list of many polymeric agents as an optional further ingredient (paragraph [0062]). Polyols such as glycerol (glycerin), propylene glycol, hexylene glycol, diethylene glycol and propylene glycol, as well as ethylene glycol, hexylene glycol, other glycols, as well as polyethylene glycol, are also mentioned as optional penetration enhancers in paragraphs [0190] and [0191].

WO 2009/019498, the disclosure of which is incorporated herein by reference, describes the use of a non-thiol reductant which does not have a pKa between 1 and 4, as a component additional to the nitrite salt and a proton source. Examples of the non-thiol reductant are stated to be iodide anion, butylated hydroquinone, tocopherol, butylated hydroxyanisole, butylated hydroxytoluene and beta-carotene. Apart from the butylated hydroquinone, the compositions of WO 2009/019498 are polyol free.

WO 2014/188174 and WO 2014/188175, the disclosures of which are incorporated herein by reference, describe a dressing system for skin lesions and a transdermal delivery system in which the proton source is a hydrogel comprising pendant carboxylic acid and sulphonate groups covalently bonded to a three-dimensional polymeric matrix. The skin contacting primary layer is a polypropylene mesh onto which the nitrite salt is imbibed. When the mesh is placed on the skin and the hydrogel is overlain on the mesh as a top layer, the reaction products of the acid and the nitrite are found to be well delivered to the skin without unacceptable skin irritation. In WO 2014/188175 an alternative skin contacting primary layer is disclosed, which is a dissolvable film formed, for example, from polyvinyl alcohols and containing the nitrite. It is taught in both references that the hydrogel may comprise glycerol, the purpose of which is not stated. However, it is well known that glycerol is added to hydrogels of this type as a plasticizer (see, for example, WO 00/06215, page 14, the disclosure of which is incorporated herein by reference). The references disclose a preference for certain hydroxyl-containing ingredients to be not present, in particular 1-thioglycerol, erythorbate, ascorbic acid and butylated hydroquinone.

US Patent Application No. 2014/0335207, the disclosure of which is incorporated herein by reference, describes a topical mixture that produces nitric oxide on mixing of a “nitrite medium” with an “acidified medium”. Specific embodiments of “nitrite medium” are individually described in paragraphs [0050] to [0055], in which the nitrite is present with one or more polyol components. The generic nitrite media described in paragraphs [0054] and [0055] contain polyols selected from glycerin, glyceryl stearate, caprylyl glycol, ethylhexylglycerin and hexylene glycol and specific embodiments described in other paragraphs contain some of the above and butylene glycol. These polyols are also components of the embodiments of the “acidified medium” described in paragraphs [0056] to [0062].

US Patent Application No. 2015/0030702, the disclosure of which is incorporated herein by reference, describes a skin dressing based on the nitrite-acid reaction. The skin dressing comprises a non-thiol reductant such as hydroquinone or butylated hydroquinone. The skin dressing may comprise a hydrogel, for example comprising hydrophilic polymers such as polyvinyl alcohol or polyethylene glycol.

US Patent Application No. 2017/0209485, the disclosure of which is incorporated herein by reference, describes an apparatus and method for topically applying nitric oxide in a foam or serum carrier. The use of glycerol and (unspecified) “glycerol-like components” as optional additives to increase surface tension and/or lower vapour pressure is described in paragraph [0070].

US Patent Application No. 2019/0134080, the disclosure of which is incorporated herein by reference, describes a composition and method for topically applying a nitric oxide generating system to skin as a foam formed from a multi-part combination comprising a first solution comprising at least one nitrite reactant and a second solution comprising at least one acidic reactant. Devices for holding, aerating and dispensing the components of the combination as a foam are also described. The use of glycerol as an optional additive to increase surface tension and/or lower vapour pressure is mentioned (paragraph [0068]).

The present invention is based on our surprising finding that nitric oxide, optionally other oxides of nitrogen and/or optionally precursors thereof (collectively referred to as NOx) can be generated more efficiently and with an enhanced reaction output than hitherto, using a proton source comprising one or more acid selected from organic carboxylic acids and organic non-carboxylic reducing acids as the nitrite salt acidifier, in the presence of one or more organic polyol. In addition, antimicrobially effective reaction products of such reaction systems using organic reducing acids as the nitrite salt acidifier are found to be deliverable at a physiologically tolerable pH, for example a pH between about 5 and about 8, with or without the use of the one or more organic polyol, making available reaction systems operating at such pHs for direct delivery as compositions with beneficial physiological activity such as in vivo antimicrobial activity. The nitric oxide generating method which underlies the present invention has been found to generate physiologically effective amounts of nitric oxide, optionally other oxides of nitrogen and/or optionally precursors thereof for an extended period of time, for example in excess of about 2 hours, for example in excess of about 5 hours, for example in excess of about 10 hours, optionally after an initial strong burst of NOx generation, leading to potentially significant uses in medicine and other applications. If the initial strong burst is not required, the administration of the reaction mixture to the subject could be done after a period of time after the initiation of the NOx generating reaction, for example about 10 minutes, 30 minutes or one hour or longer after the initiation of the NOx generating reaction.

### SUMMARY OF THE INVENTION

The present invention provides systems, methods, combinations, kits and compositions for generating nitric oxide and optionally other oxides of nitrogen and/or optionally precursors thereof. The systems, methods, combinations, kits and compositions include as reactants one or more nitrite salt and a proton source comprising one or more acid selected from organic carboxylic acids and organic non-carboxylic reducing acids. The systems, methods, combinations, kits and compositions further include one or more organic polyol. The use of reducing acids (that is:

carboxylic reducing acids and non-carboxylic reducing acids) allows nitric oxide and optionally other oxides of nitrogen and/or optionally precursors thereof to be generated at pHs somewhat higher than 4, for example in the range 5 to 8. The present invention further provides systems, methods, combinations, kits and compositions for antimicrobial use, where the one or more organic polyol is optional and the reaction is performed at a starting pH of the proton source in the range 5 to 8.

According to a first aspect, the present invention provides a method for generating nitric oxide, optionally other oxides of nitrogen and/or optionally precursors thereof, comprising reacting one or more nitrite salt with a proton source comprising one or more acid selected from organic carboxylic acids and organic non-carboxylic reducing acids under reaction conditions suitable to generate nitric oxide, optionally other oxides of nitrogen and/or optionally precursors thereof, wherein the reaction is performed in the presence of one or more organic polyol;

characterised by one or more of the following:
(a) the one or more organic polyol is present in a reaction output enhancing amount;
(b) the proton source is not solely a hydrogel comprising pendant carboxylic acid groups covalently bonded to a three-dimensional polymeric matrix;
(c) the one or more organic polyol is not solely glycerol;
(d) the one or more organic polyol is not solely glycerol when one or more viscosity increasing agent is used;
(e) the one or more organic polyol is not solely glycerol when one or more plasticizer is used; (0 the one or more organic polyol is not solely polyvinyl alcohol;
(g) the one or more organic polyol is not solely polyvinyl alcohol when one or more viscosity increasing agent is used;
(h) any one or more of (b) to (g) above, wherein the words “is not solely” are replaced by “does not comprise”;
(i) the one or more organic polyol is not solely propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol;
(j) the one or more organic polyol does not comprise propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol.

The nitric oxide, optionally other oxides of nitrogen and/or optionally precursors thereof prepared by the method according to the first aspect of the invention constitute a second aspect of the present invention.

According to a third aspect, the present invention provides a method of enhancing the output of the reaction of one or more nitrite salt with a proton source to generate nitric oxide, optionally other oxides of nitrogen and/or optionally precursors thereof, comprising using a proton source comprising one or more acid selected from organic carboxylic acids and organic non-carboxylic reducing acids and performing the reaction in the presence of a reaction output enhancing amount of one or more organic polyol. The enhancement of the output of the reaction is in comparison with a reaction performed under the same conditions but without the one or more organic polyol.

According to a fourth aspect, the present invention provides the use of one or more organic polyol in a reaction mixture to enhance the output of the reaction, in the reaction mixture, of one or more nitrite salt with a proton source to generate nitric oxide, optionally other oxides of nitrogen and/or optionally precursors thereof, wherein the proton source comprises one or more acid selected from organic carboxylic acids and organic non-carboxylic reducing acids. The enhancement of the output of the reaction is in comparison with a reaction performed under the same conditions but without the one or more organic polyol.

According to a fifth aspect, the present invention provides a combination, kit or composition for generating nitric oxide, optionally other oxides of nitrogen and/or optionally precursors thereof by reaction of one or more nitrite salt with a proton source, the combination, kit or composition comprising:
(i)one or more nitrite salt;
(ii) a proton source comprising one or more acid selected from organic carboxylic acids and organic non-carboxylic reducing acids; and
(iii) one or more organic polyol;

characterised by one or more of the following:
(a) the one or more organic polyol is present in a reaction output enhancing amount;
(b) the proton source is not solely a hydrogel comprising pendant carboxylic acid groups covalently bonded to a three-dimensional polymeric matrix;
(c) the one or more organic polyol is not solely glycerol;
(d) the one or more organic polyol is not solely glycerol when one or more viscosity increasing agent is used;
(e) the one or more organic polyol is not solely glycerol when one or more plasticizer is used;
(f) the one or more organic polyol is not solely polyvinyl alcohol;
(g) the one or more organic polyol is not solely polyvinyl alcohol when one or more viscosity increasing agent is used;
(h) any one or more of (b) to (g) above, wherein the words “is not solely” are replaced by “does not comprise”;
(i) the one or more organic polyol is not solely propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol;
(j) the one or more organic polyol does not comprise propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol.

When the proton source comprises a hydrogel comprising pendant carboxylic acid groups covalently bonded to a three-dimensional polymeric matrix and the combination or kit comprises two or more separate compositions, it is preferred that the one or more polyol is not present in the separate compositions in direct contact or admixture with the hydrogel.

The chemical substances of the combination, kit or composition of the fifth aspect of the present invention may, for example, consist essentially of the components (i), (ii) and (iii) stated above and optionally water and/or a pH buffer. The expression “consists essentially of” may, for example, permit minor amounts of one or more additional component to be present provided that the effect of the components (i), (ii) and (iii) stated above and optionally water and/or a pH buffer is not adversely affected. The total amount of such one or more additional component may suitably be less than about 20% by weight or volume of the combination, of the chemical ingredients of the kit, or of the composition, for example less than about 15% by weight or volume, for example less than about 10% by weight or volume, for example less than about 5% by weight or volume.

The chemical substances of the combination, kit or composition may, for example, consist of the components (i), (ii) and (iii) stated above and optionally water and/or a pH buffer and/or one or more additional component in an amount of less than about 20% by weight or volume of the combination, of the chemical ingredients of the kit, or of the composition, for example less than about 15% by weight or volume, for example less than about 10% by weight or volume, for example less than about 5% by weight or volume.

According to a sixth aspect, the present invention provides a method of preparing a combination, kit or composition comprising:
(i)one or more nitrite salt;
(ii) a proton source comprising one or more acid selected from organic carboxylic acids and organic non-carboxylic reducing acids; and
(iii) one or more organic polyol;
which comprises bringing components (i), (ii) and (iii) into mutual proximity to form the combination or kit or into admixture to form the composition;

characterised by one or more of the following:
(a) the one or more organic polyol is present in a reaction output enhancing amount;
(b) the proton source is not solely a hydrogel comprising pendant carboxylic acid groups covalently bonded to a three-dimensional polymeric matrix;
(c) the one or more organic polyol is not solely glycerol;
(d) the one or more organic polyol is not solely glycerol when one or more viscosity increasing agent is used;
(e) the one or more organic polyol is not solely glycerol when one or more plasticizer is used;
(f) the one or more organic polyol is not solely polyvinyl alcohol;
(g) the one or more organic polyol is not solely polyvinyl alcohol when one or more viscosity increasing agent is used;
(h) any one or more of (b) to (g) above, wherein the words “is not solely” are replaced by “does not comprise”;
(i) the one or more organic polyol is not solely propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol;
(j) the one or more organic polyol does not comprise propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol.

The expression “combination” used herein refers to separate substances or compositions (referred to as “components”) which are brought into proximity and used together. The bringing of the components into proximity can be achieved in multiple stages, whereby some but not all of the components are initially brought together into a sub-combination or partial combination, which is subsequently brought into proximity with one or more further components or other sub-combinations or partial combinations. “Proximity” can include an intimate admixture, solution or suspension, or can signify close physical proximity which does not amount to intimate admixture, solution or suspension, for example in separate containers in a kit in which the components are provided together for convenient later use. For example, a nitrite component and a proton source component, comprising respectively the one or more nitrite salt (or some of them) and the one or more acid selected from organic carboxylic acids and organic non-carboxylic reducing acids (or some of them), may be stored separately or in separate containers of a kit, and brought together for use by mixing to initiate the NOx generating reaction. The one or more organic polyol may be provided in one or both of the nitrite component and the proton source component, or may be provided separately in an organic polyol component which is also mixed in when the NOx generating reaction is initiated. Any one or more of the components may itself be present in multiple parts and in multiple containers. The combination may be brought into proximity in such a way that the NOx generating reaction is initiated immediately, for example because the nitrite salt and the proton source are in the same solution and are therefore able to react. Alternatively, the combination may be brought into proximity in such a way that the NOx generating reaction is not initiated immediately but requires one or more further step or action to take place before initiation, for example because the nitrite salt and the proton source are in dry powdered admixture or are present as encapsulated particles which require water (e.g from mucosal membranes contacted by the combination) before the NOx generating reaction will start.

In embodiments, the first to sixth aspects of the invention may independently of each other be characterised by the above-mentioned feature (a) only, or by feature (b) only, or by feature (c) only, or by feature (d) only, or by feature (e) only, or by feature (f) only, or by feature (g) only, or by feature (h) as it refers to (b) only, or by feature (h) as it refers to (c) only, or by feature (h) as it refers to (d) only, or by feature (h) as it refers to (e) only, or by feature (h) as it refers to (f) only, or by feature (h) as it refers to (g) only, or by features (a) and (b) only, or by feature (h) as it refers to features (a) and (b), or by features (a) and (c) only, or by feature (h) as it refers to features (a) and (c), or by features (a) and (d) only, or by feature (h) as it refers to features (a) and (d), or by features (a) and (e) only, or by feature (h) as it refers to features (a) and (e), or by features (a) and (f) only, or by feature (h) as it refers to features (a) and (f), or by features (a) and (g) only, or by feature (h) as it refers to features (a) and (g), or by features (b) and (c) only, or by feature (h) as it refers to features (b) and (c), or by features (b) and (d) only, or by feature (h) as it refers to features (b) and (d), or by features (b) and (e) only, or by feature (h) as it refers to features (b) and (e), or by features (b) and (f) only, or by feature (h) as it refers to features (b) and (f), or by features (a), (b), (c) and (f) only, or by feature (h) as it refers to features (a), (b), (c) and (f), or by all of features (a) to (g), or by features (a) and (b) together with feature (h) as it refers to all of features (c) to (g).

In other embodiments, the first to sixth aspects of the invention may independently of each other be characterised by the above-mentioned features (c), (f) and (i) only, or by features (c), (f) and (j) only, or by features (i) and (h) as it refers to features (c) and (f), or by features (j) and (h) as it refers to features (c) and (f), or by features (d), (g) and (i) only, or by features (d), (g) and (j) only, or by features (i) and (h) as it refers to features (d) and (g), or by features (j) and (h) as it refers to features (d) and (g), or by features (e), (f) and (i) only, or by features (e), (f) and (j) only, or by features (i) and (h) as it refers to features (e) and (f), or by features (j) and (h) as it refers to features (e) and (f).

It is preferred that the first to sixth aspects of the invention are characterised either by all of features (a) to (g), or by features (a) and (b) together with feature (h) as it refers to all of features (c) to (g), or by features (c), (f) and (i) only, or by features (c), (f) and (j) only, or by features (i) and (h) as it refers to features (c) and (f), or by features (j) and (h) as it refers to features (c) and (f), or by features (d), (g) and (i) only, or by features (d), (g) and (j) only, or by features (i) and (h) as it refers to features (d) and (g), or by features (j) and (h) as it refers to features (d) and (g), or by features (e), (f) and (i) only, or by features (e), (f) and (j) only, or by features (i) and (h) as it refers to features (e) and (f), or by features (j) and (h) as it refers to features (e) and (f). It will be noted that features (d), (e) and (g) are redundant when features (c) and (f) characterise the invention; in that case, features (d), (e) and (g) (or feature (h) as it refers to features (d), (e) and (g)) may be omitted from the list and considered as examples of the characterising features (c) and (f) (or feature (h) as it refers to features (c) and (f)).

The expression “reaction output enhancing amount of one or more organic polyol” used herein means that the amount of the one or more organic polyol causes the amount and/or output time period of at least one of nitric oxide, optionally other oxides of nitrogen and/or optionally precursors thereof from the NOx generating reaction to be higher than if the reaction had been performed under the same conditions but without the one or more organic polyol. The expression “amount” means particularly the total mass of evolved gaseous nitric oxide, per gram of nitrite available to react in the starting reaction system. The experimental work underlying the present invention has measured the amount of evolved gaseous nitric oxide, optionally also other gases, and has found these to be enhanced. From this it is believed that the total mass of generated NOx is enhanced by the present invention, so that the expression “amount” is also understood to include the total mass of nitric oxide which passes into solution in the reaction mixture as well as the total mass of NOx reaction product. The expression “output time period” means particularly the length of time over which at least one of gaseous nitric oxide, optionally also other gases, is evolved in the reaction before the reaction is completed. For the same reason as explained above in the discussion of the phrase “reaction output enhancing amount of one or more organic polyol”, it is believed that the phrase “output time period” also includes the length of time over which nitric oxide passes into solution in the reaction mixture as well as the length of time over which NOx reaction product is generated. As is well known, eventually the nitrite salt is exhausted by the reaction with the proton source, the pH— which rises during the NOx generating reaction—reaches its maximum and the reaction stops. It is preferred that the method of the first aspect of the present invention enhances the yield of the NOx generating reaction, particularly but not exclusively the amount of NO produced, for example the amount of gaseous NO produced, by at least about 5%, for example at least about 10%, for example at least about 25%, for example up to a degree of enhancement by about 150%, for example up to a degree of enhancement by about 125%, for example up to a degree of enhancement by about 100%, for example up to a degree of enhancement by about 75%. It is preferred that the method of the first aspect of the present invention enhances the length of time over which at least one of nitric oxide, optionally other oxides of nitrogen and/or optionally precursors thereof, preferably nitric oxide, is evolved in the reaction before the reaction is completed by at least about 5%, for example at least about 10%. Using the present invention, the time period over which at least one of nitric oxide, optionally other oxides of nitrogen and/or optionally precursors thereof, preferably nitric oxide and most preferably gaseous nitric oxide, is evolved—and particularly is evolved in effective amounts—can be enhanced to at least about 2 hours, for example at least about 5 hours, for example up to or more than about 10 hours. This degree of time enhancement of the evolution of nitric oxide can represent, for example, up to or more than a degree of enhancement by about 150% of the period for evolution of the same amount of nitric oxide without the use of the polyol component, for example up to a degree of enhancement by about 125%, for example up to a degree of enhancement by about 100%, for example up to a degree of enhancement by about 75%.

The generation of the nitric oxide, optionally other oxides of nitrogen and/or optionally precursors may be for any purpose. Both therapeutic and non-therapeutic purposes are exemplified and discussed below.

According to a seventh aspect, the present invention provides a therapeutic or non-therapeutic method of delivering nitric oxide, optionally other oxides of nitrogen and/or optionally precursors thereof to a target location, for example any cell, organ, surface, structure, subject, or an internal space therewithin, which comprises (a) administering to the target location or to the vicinity thereof, a combination or composition according to the fifth aspect of the invention; or (b) using a method according to the first or third aspect of the invention, or performing a use according to the fourth aspect of the invention, or using a combination, kit or composition according to the fifth aspect of the invention to generate nitric oxide, optionally other oxides of nitrogen and/or optionally precursors thereof and delivering the nitric oxide, optionally other oxides of nitrogen and/or optionally precursors thereof thereby generated to the target location or vicinity thereof; or (c) delivering the nitric oxide, optionally other oxides of nitrogen and/or optionally precursors thereof according to the second aspect of the invention to the target location or vicinity thereof.

The method of the seventh aspect of the present invention may, for example, be a method of treating a microbial infection in a subject in need thereof. The subject may, for example, be a human subject or other mammalian subject. The microbial infection may, for example, be bacterial, viral, fungal, microparasitical or any combination thereof.

The method of the seventh aspect of the present invention may, for example, be a method of vasodilation performed on a subject. The subject may, for example, be a human subject or other mammalian subject.

The method of the seventh aspect of the present invention may, for example, be an antimicrobial method. The antimicrobial method may be to reduce the number of microbes, for example bacteria, viruses, fungal cells and/or microparasitic organisms, at a locus, to prevent proliferation thereof, or to restrict the rate of proliferation thereof. Microbes targeted by such a method may, for example, be planktonic cells or particles or present as a biofilm or other colony. Any population of microbes, whether planktonic or not, targeted by the present invention can consist of one microbial species or strain or can comprise more than one species or strain.

According to an eighth aspect, the present invention provides a combination, kit or composition according to the fifth aspect of the invention, or nitric oxide, optionally other oxides of nitrogen and/or optionally precursors thereof according to the second aspect of the invention, for use in therapy.

The combination, kit or composition or the nitric oxide, optionally other oxides of nitrogen and/or optionally precursors thereof for use according to the eighth aspect of the invention may, for example, be for use in a therapeutic method of delivering nitric oxide, optionally other oxides of nitrogen and/or optionally precursors thereof to a subject, or an internal space therewithin, which comprises (a) administering to the subject or internal space, or to the vicinity thereof, a combination or composition according to the fifth aspect of the invention; or (b) using a method according to the first or third aspect of the invention, or performing a use according to the fourth aspect of the invention, or using a combination, kit or composition according to the fifth aspect of the invention to generate nitric oxide, optionally other oxides of nitrogen and/or optionally precursors thereof and delivering the nitric oxide, optionally other oxides of nitrogen and/or optionally precursors thereof thereby generated to the subject or internal space, or vicinity thereof; or (c) delivering the nitric oxide, optionally other oxides of nitrogen and/or optionally precursors thereof according to the second aspect of the invention to the subject or internal space, or vicinity thereof.

In accordance with the present invention, we have found surprisingly that a good antimicrobial activity in terms of biostatic and biocidal effect, evidenced by up to 100% killing of M. abscessus after 3 days and/or killing of M. tuberculosis, H1N1 Influenza virus, SARS-CoV virus and SARS-CoV-2 virus, is also provided when the proton source is citric acid (an organic carboxylic acid) or ascorbic acid (an organic non-carboxylic reducing acid) having an initial pH in the range of 5 to 8. The expression “initial pH” herein refers to the pH of an initially formed aqueous solution of the proton source, including any desired pH buffer, before other components of the reaction mixture are present that will affect that initial pH. This antimicrobial effect is not dependent on the presence of one or more organic polyol, although it appears to be enhanced by the presence of one or more organic polyol, for example mannitol or sorbitol. The finding of a strong antimicrobial effect from the NOx generating reaction products where the acid (e.g. citric or ascorbic acid) has an initial pH in the range of 5 to 8 is especially surprising, and offers promising applications in the treatment of respiratory tract and lung infections including those which are difficult to treat and/or resistant to antibiotics, including tuberculosis, multi-drug resistant tuberculosis and non-tuberculosis mycobacterium infections. Treatments of such infections can be proposed via inhalation of nebulised aqueous compositions containing the reaction mixture or components or precursors thereof at a pH in the range of 5 to 8. Treatments of infections comprising multiple pathogens, potentially including pathogens from more than one of the groups bacteria, viruses, fungi and parasites, known as “broad spectrum” treatments (including therapeutic and/or prophylactic treatments as well as in vitro treatments of animate and inanimate surfaces and spaces to prevent spread of pathogens) are also enabled by the present invention.

According to a ninth aspect, the present invention provides a modification of the antimicrobial method according to the seventh aspect, which comprises (a) administering to the microbes to be targeted, or to the vicinity thereof, or to a subject infected with microbes or an internal space of such a subject, a combination or composition according to the fifth aspect of the invention; or (b) using a method according to the first or third aspect of the invention, or performing a use according to the fourth aspect of the invention, or using a combination, kit or composition according to the fifth aspect of the invention to generate nitric oxide, optionally other oxides of nitrogen and/or optionally precursors thereof and delivering the nitric oxide, optionally other oxides of nitrogen and/or optionally precursors thereof thereby generated to the microbes to be targeted, or to the vicinity thereof, or to a subject infected with microbes or an internal space of such a subject; or (c) delivering the nitric oxide, optionally other oxides of nitrogen and/or optionally precursors thereof according to the second aspect of the invention to the microbes to be targeted, or to the vicinity thereof, or to a subject infected with microbes or an internal space of such a subject;

provided that the initial pH of an aqueous solution of the proton source including any desired buffer before other components of the NOx generating reaction mixture are present that will affect the pH, or the pH of the reaction mixture at the start of the reaction with the one or more nitrite salt, is in the range of 5 to 8, and the one or more polyol is optional and may be omitted.

In performing the method according to the ninth aspect of the present invention, the combination, kit or composition according to the fifth or eighth aspect of the invention may be used to generate the nitric oxide, optionally other oxides of nitrogen and/or optionally precursors thereof; provided that the initial pH of an aqueous solution of the proton source including any desired buffer before other components of the NOx generating reaction mixture are present that will affect the pH, or the pH of the reaction mixture at the start of the reaction with the one or more nitrite salt, is in the range of 5 to 8, and the one or more polyol is optional and may be omitted.

The method of the ninth aspect of the present invention may, for example, be a method of treating a microbial infection in a subject in need thereof. The subject may, for example, be a human subject or other mammalian subject. The microbial infection may, for example, be bacterial, viral, fungal, microparasitic infection or any combination thereof. The microbial infection may be on the skin of the subject, including mucosae. The microbial infection may be in an internal space of the subject, for example in the nose, mouth, respiratory tract, lungs of the subject or lining of the lung pleura.

The components and mixtures used in all aspects of the present invention to be administered to the human or animal body, as well as any carriers and excipients to be administered to the human or animal body, will preferably be biocompatible and/or pharmaceutically acceptable to minimise irritation and inflammation of tissues on administration.

The combinations, kits and compositions according to the invention may be stored and used with a variety of suitable apparatus and devices, which will be described in more detail below. The methods according to the invention may suitably be performed using such apparatus and devices, as will be described in more detail below.

All embodiments, examples and preferences described specifically in respect of any one or more aspect of the present invention are to be understood as being applicable to any one or more other aspect(s) of the invention. In addition, any method or use according to one aspect of the invention may if desired be performed using a combination, kit or composition according to any other aspect.

### DETAILED DESCRIPTION

The aspects of the present invention are now described in detail with reference to particular embodiments. The particular embodiments described below may apply to any of the aspects of the present invention, unless clearly incompatible with such an aspect. The particular embodiments are also combinable with each and every other particular embodiment unless incompatible to do so.

Nitrite Salts and Nitrite Component

Aspects of the present invention involve the use of one or more nitrite salt. In the following the term “nitrite component” covers the one or more nitrite salt per se and any component of the reaction system for generating nitric oxide, optionally other oxides of nitrogen and/or optionally precursors thereof that contains the one or more nitrite salt.

The choice of nitrite salt is not particularly limited. Specific examples of nitrite salts that may be used in the compositions of the present invention include alkali metal nitrites or alkaline earth metal nitrites. In some embodiments, the one or more nitrite salt is selected from LiNO₂, NaNO₂, KNO_{2,} RbNO₂, CsNO₂, FrNO₂, AgNO₂, Be(NO₂)₂, Mg(NO₂)₂, Ca(NO₂)₂, Sr(NO₂)₂, Mn(NO₂)₂, Ba(NO₂)₂, Ra(NO₂)₂ and any mixture thereof.

In particular embodiments, the nitrite salt is NaNO₂ or KNO₂. In one embodiment, the nitrite salt is NaNO₂.

In one embodiment, the nitrite component it may be provided for use in the invention in dry form, optionally in particulate form such as a powder. If desired, the nitrite component may be encapsulated or microencapsulated, e.g. for the purpose of controlling or delaying the reaction between the one or more nitrite salt and the proton source. The dry form and/or the encapsulation may assist the storage of the nitrite component, whether alone or in admixture with other components of the reaction to generate the nitric oxide according to the invention. Still further, the dry form and/or the encapsulation may assist the incorporation of the nitrite component, whether alone or in admixture with other components of the reaction to generate the nitric oxide according to the invention, into small objects such as medical devices. Such objects include, for example, wound dressings, bandages, vascular and other stents, catheters, pacemakers, defibrillators, heart assist devices, artificial valves, electrodes, orthopaedic screws and pins, and other thin medical and/or implantable articles and inhalers (handheld and nebulizer). Please see the section below headed “Optional Encapsulation (e.g. Microencapsulation) of Components” for more details.

If desired, the nitrite component, optionally encapsulated or microencapsulated, can be present as a dry powder or crystals, or in association with a gel or other carrier system, for example an aqueous carrier, e.g. as an aqueous gel or solution thereof. A nitrite component in dry or powder form may conveniently be made up into solution before use by addition of water. The molarity of nitrite ion in such a nitrite solution before (for example, immediately before) addition of any other components of the NOx generating reaction mixture, and in particular before (for example, immediately before) acidification, may be in the range of about 0.001 M to about 5 M. In some embodiments, the molarity of nitrite ion in the nitrite solution before (for example, immediately before) addition of any other components of the NOx generating reaction mixture, and in particular before (for example, immediately before) acidification is in the range of about 0.01 M to about 2 M. In some embodiments, the molarity of nitrite ion in the nitrite solution before (for example, immediately before) addition of any other components of the NOx generating reaction mixture, and in particular before (for example, immediately before) acidification is in the range of about 0.1 M to about 2 M. In more particular embodiments, the molarity of nitrite ion in the nitrite solution before (for example, immediately before) addition of any other components of the NOx generating reaction mixture, and in particular before (for example, immediately before) acidification is in the range of about 0.2 M to about 1.6 M. In embodiments, the molarity of nitrite ion in the nitrite solution before (for example, immediately before) addition of any other components of the NOx generating reaction mixture, and in particular before (for example, immediately before) acidification can be in the range of 0.8 to 1.2 M. For example, the molarity of nitrite ion in the nitrite solution before (for example, immediately before) addition of any other components of the NOx generating reaction mixture, and in particular before (for example, immediately before) combination with the organic carboxylic acid component may be about 0.8 M, about 0.9 M, about 1.0 M, about 1.1 M, about 1.2 M, about 1.5 M or about 1.7 M.

It is to be noted that the act of combining two or more precursor solutions of the NOx generating reaction mixture will cause a dilution of the concentration of each solute or combination of solutes in each solution, as is well known to those skilled in the art. For example, the act of mixing equal volumes of two 1 M solutions of solutes A and B causes the concentration of A to change to 0.5 M and the concentration of B to change to 0.5 M. Unless otherwise stated or implied, the concentration of nitrite salt described herein is its concentration in an initial solution before (for example, immediately before) addition of any other components of the NOx generating reaction mixture that are added as liquids, e.g. solutions. The actual concentration in the NOx generating reaction mixture can readily be derived knowing the components of the reaction mixture and how it was prepared.

If desired, the nitrite component, whether in dry form or in a carrier liquid, can include the one or more polyol or some of such polyols.

If the nitrite component is desired to be stored in a gel or other carrier system, for example an aqueous carrier, e.g. as an aqueous gel or solution, it is preferred that the system containing the nitrite is buffered to a suitable pH to prevent degradation of the nitrite during storage. A pH of about 6-9, for example about 7, is preferred.

It is preferred that the nitrite component is not brought into contact with the proton source until it is desired to generate the nitric oxide, optionally other oxides of nitrogen and/or optionally precursors thereof. For this reason, the nitrite component is preferably held in a reservoir or container of a kit, apparatus or device. However, it may alternatively be possible for dry components of the nitrite component, the proton source and the one or more polyol to be held as a dry composition, e.g. a particulate mixture, and for the reaction to be initiated by the simple addition of water or another suitable solvent or liquid carrier.

The nitrite salt may be a pharmaceutically acceptable grade of nitrite salt. In some embodiments, the nitrite salt is pharmacopoeia grade. In other words, the nitrite salt may adhere to one or more active pharmacopoeia monographs for the nitrite salt. For example, the nitrite salt may adhere to the monograph of the nitrite salt of one or more of the United States Pharmacopoeia (USP), European Pharmacopoeia or Japanese Pharmacopoeia.
In particular embodiments, the nitrite salt used has one or more of the following limitations on its characteristics: (i) the nitrite salt contains no more than about 0.02%, about 0.01% or about 0.001% by weight of sodium carbonate;
(ii) the nitrite salt contains no more than about 10 ppm (0.001% by weight) of an anti-caking agent, such as sodium alkyl-naphthalene sulfonate;
(iii) the nitrite salt is a white to off-white solid;
(iv) the nitrite salt has a positive identification for the cation determined according to the relevant method in the relevant USP;
(v) the nitrite salt has a positive identification test for nitrite determined according to the relevant method in the relevant USP;
(vi) the nitrite salt contains no less than about 97% or no less than 98% by weight of the nitrite salt and/or no more than 102% or no more than 101% by weight of the nitrite salt, optionally as determined by the relevant USP calorimetric assay, for example, as determined by ion chromatography, such as ion chromatography coupled with suppressed conductivity detection;
(vii) the nitrite salt has a pH between about 7 and about 9 or between about 8 and about 9 when measured in a 10% solution at 25° C., optionally measured according to the relevant USP and/or using a pH meter;
(viii) the nitrite salt has a loss on drying of no more than about 0.25% or about 0.01% by weight;
(ix) the nitrite salt has a water content of no more than about 0.5% by weight, optionally as determined by the Karl Fischer method;
(x) the heavy metal content in the nitrite salt is no more than about 10 ppm of a heavy metal, optionally the heavy metal content in the nitrite salt is no more than about 10 ppm; (xi) the nitrite salt contains no more than about 0.4% by weight of a nitrate salt, optionally no more than about 0.4% by weight sodium nitrate when the nitrite salt is sodium nitrite and no more than about 0.4% by weight potassium nitrate when the nitrite salt is potassium nitrite;
(xii) the nitrite salt contains no more than about 0.005% or about 0.001% by weight of insoluble matter;
(xiii) the nitrite salt contains no more than about 0.005% by weight of chloride;
(xiv) the nitrite salt contains no more than about 0.01% by weight of sulphate;
(xv) the nitrite salt contains no more than about 0.001% by weight of iron;
(xvi) the nitrite salt contains no more than about 0.01% by weight of calcium;
(xvii) the nitrite salt contains no more than about 0.005% or about 0.001% by weight of potassium when the nitrite salt is not potassium nitrite or no more than about 0.005% or about 0.001% by weight of sodium when the nitrite salt is not sodium nitrite;
(xviii) the nitrite salt contains no more than about 0.1% by weight, no more than about 5000 ppm, no more than about 1000 ppm, no more than about 500 ppm, no more than about 100 ppm or no more than about 10 ppm of organic volatile compounds;
(xix) the nitrite salt contains no more than about 0.1% by weight, no more than about 5000 ppm, no more than about 1000 ppm, no more than about 500 ppm, no more than about 100 ppm or no more than about 10 ppm of ethanol;
(xx) the nitrite salt contains no more than about 3000 ppm, no more than about 1000 ppm, no more than about 500 ppm, no more than about 100 ppm or no more than about 10 ppm of methanol;
(xxi) the nitrite salt contains no more than about 50 ppm, no more than about 25 ppm, no more than about 20 ppm, no more than about 10 ppm, no more than about 7.9 ppm, no more than about 8 ppm, no more than about 6 ppm, no more than about 5.6 ppm, or no more than about 2.5 ppm of non-volatile organic carbon;
(xxii) the nitrite salt contains no more than about 0.05 ppm of mercury;
(xxiii) the nitrite salt contains no more than about 2 ppm or 0.2 ppm of aluminium;
(xxiv) the nitrite salt contains no more than about 3 ppm or 1 ppm of arsenic;
(xxv) the nitrite salt contains no more than about 0.003% or 0.001% by weight of selenium;
(xxvi) the total aerobic count of microbial load in the nitrite salt is no more than about 100 CFU/g;
(xxvii) the total yeast and mold count in the nitrate salt is no more than about 20 CFU/g;
(xxviii) the nitrite salt contains no more than about 0.25 EU/mg or 0.018 EU/mg of bacterial endotoxins; and
(xxix) the nitrite salt contains less than about 0.1 ppm of a phosphate salt, such as sodium phosphate, disodium hydrogen phosphate or trisodium phosphate, and preferably the nitrite salt contains no detectable amount of phosphate salt.

In certain embodiments, the nitrite salt has two or more of the characteristics of (i) to (xxix). In further embodiments, the nitrite salt has five or more of the characteristics of (i) to (xxix). In yet further embodiments, the nitrite salt has ten or more of the characteristics of (i) to (xxix). In even further embodiments, the nitrite salt has fifteen or more of the characteristics of (i) to (xxix). In some embodiments, the nitrite salt has twenty or more of the characteristics of (i) to (xxix). In a particular embodiment, the nitrite salt has all of the characteristics of (i) to (xxix). In a more particular embodiment, the nitrite salt is sodium nitrite having all of the characteristics of (i) to (xxix).

In some embodiments the nitrite salt contains in the range of about 97% to about 101% by weight of the nitrite salt, optionally as determined by the relevant USP calorimetric assay, for example, as determined by ion chromatography, such as ion chromatography coupled with suppressed conductivity detection. In alternative embodiments nitrite salt contains in the range of about 98% to about 102% by weight of the nitrite salt, optionally as determined by the relevant USP calorimetric assay, for example, as determined by ion chromatography, such as ion chromatography coupled with suppressed conductivity detection

In particular embodiments the nitrite salt has the following characteristics:
(i) the nitrite salt contains no more than about 0.02% by weight of sodium carbonate;
(ii) the nitrite salt contains no more than about 10 ppm of an anti-caking agent;
(vi) the nitrite salt contains no less than 97% by weight of the nitrite salt and no more than 101% by weight of the nitrite salt as determined by USP calorimetric assay;
(viii) the nitrite salt has a loss on drying of no more than about 0.25% by weight;
(ix) the nitrite salt has a water content of no more than about 0.5% by weight;
(x) the heavy metal content in the nitrite salt is no more than about 10 ppm;
(xi) the nitrite salt contains no more than about 0.4% by weight of a nitrate salt;
(xii) the nitrite salt contains no more than about 0.005% by weight of insoluble matter; (xiii) the nitrite salt contains no more than about 0.005% by weight of chloride;
(xiv) the nitrite salt contains no more than about 0.01% by weight of sulphate;
(xv) the nitrite salt contains no more than about 0.001% by weight of iron;
(xvi) the nitrite salt contains no more than about 0.01% by weight of calcium;
(xviii) the nitrite salt contains no more than about no more than about 5000 ppm, no more than about 1000 ppm, no more than about 500 ppm, no more than about 100 ppm or no more than about 10 ppm of organic volatile compounds;
(xxi) the nitrite salt contains no more than about 10 ppm or no more than about 2.5 ppm of non-volatile organic carbon;
(xxii) the nitrite salt contains no more than about 0.05 ppm of mercury;
(xxiii) the nitrite salt contains no more than about 2 ppm of aluminium;
(xxiv) the nitrite salt contains no more than about 3 ppm of arsenic;
(xxv) the nitrite salt contains no more than about 0.003% by weight of selenium;
(xxvi) the total aerobic count of microbial load in the nitrite salt is no more than about 100 CFU/g;
(xxvii) the total yeast and mold count in the nitrate salt is no more than about 20 CFU/g; and
(xxviii) the nitrite salt contains no more than about 0.25 EU/mg of bacterial endotoxins.

In these embodiments, the nitrite salt may be sodium nitrite and contain no more than about 0.005% by weight of potassium. Preferably the sodium nitrite also has one or more of the following limitations:
(iii) the sodium nitrite is a white to off-white solid;
(iv) the sodium nitrite has a positive identification for sodium determined according to the relevant method in the relevant USP;
(v) the sodium nitrite has a positive identification test for nitrite determined according to the relevant method in the relevant USP;
(vii) the sodium nitrite has a pH between about 7 and about 9 or between about 8 and about 9 when measured in a 10% solution at 25° C., optionally measured according to the relevant USP and/or using a pH meter;
(xix) the sodium nitrite contains no more than about 0.1% by weight, no more than about 5000 ppm, no more than about 1000 ppm, no more than about 500 ppm, no more than about 100 ppm or no more than about 10 ppm of ethanol;
(xx) the nitrite salt contains no more than about 3000 ppm, no more than about 1000 ppm, no more than about 500 ppm, no more than about 100 ppm or no more than about 10 ppm of methanol; and
(xxix) the nitrite salt contains less than about 0.1 ppm of a phosphate salt, such as sodium phosphate, disodium hydrogen phosphate or trisodium phosphate, and preferably the nitrite salt contains no detectable amount of phosphate salt.

The characteristics of (i) to (xxix) may be determined according to the relevant method in USP XXXII (2009). Methods for determining the characteristics of (i) to (xxix) are provided in WO 2010/093746, the disclosure of which is incorporated herein by reference in its entirety. Methods of preparing sodium nitrite having one or more of the characteristics of (i) to (xxix) are also described in WO 2010/093746.

Proton Sources Comprising One or More Organic Carboxylic Acid and Proton Source Component

Aspects of the present invention involve a proton source comprising one or more acid selected from organic carboxylic acids and organic non-carboxylic reducing acids. In the following the term “proton source component” covers the proton source per se and any component of the reaction system for generating nitric oxide, optionally other oxides of nitrogen and/or optionally precursors thereof that contains the proton source.

In this section, the organic carboxylic acids will be exemplified in more detail.

The expression “organic carboxylic acid” herein refers to any organic acid which contains one or more—COOH group in the molecule. An organic carboxylic acid may be straight-chain or branched. The carboxylic acid may be saturated or unsaturated. The carboxylic acid may be aliphatic or aromatic. The carboxylic acid may be acyclic or cyclic. The carboxylic acid may be a vinylogous carboxylic acid.

The organic carboxylic acid may carry one or more substituents, for example one or more hydroxyl group. Examples of hydroxyl-substituted organic carboxylic acids which may be used in the present invention include α-hydroxy-carboxylic acids, β-hydroxy-carboxylic acids and γ-hydroxy-carboxylic acids.

The one or more organic carboxylic acid, or each of them if more than one, should preferably have a pKa₁ less than about 7, more preferably less than 7.0.

The one or more carboxylic acid may be, comprise or consist of one or more reducing carboxylic acid.

The carboxylic acid may be an acid hydrogel containing pendant—COOH groups covalently attached to the polymer molecules forming the three-dimensional polymeric matrix of the hydrogel. Examples of such carboxylic acid containing hydrogels are described, for example, in WO 2007/007115, WO 2008/087411, WO 2008/087408, WO 2014/188174 and WO 2014/188175 and in the documents referred to therein, the disclosures of all of which are incorporated herein by reference. Such hydrogels typically comprise pendant carboxylic acid and sulphonyl groups in acid or salt form covalently bonded to a three-dimensional polymeric matrix. For further discussion please see the section headed “Other Reservoirs for the Components: Hydrogels” below.

Nevertheless, it is generally preferred that at least one of the one or more acid selected from organic carboxylic acids and organic non-carboxylic reducing acids is not covalently bonded to a polymer or macromolecule, for example a polymer or macromolecule forming a three-dimensional polymeric or macromolecular matrix of the hydrogel. Without wishing to be bound by theory, the evidence-for example the evidence of dependence of the effect on the stereoisomerism of the polyol(s), discussed below in the section headed “Organic Polyols”—suggests that the effect of the invention to enhance the output of the reaction of the one or more nitrite salt with the proton source is achieved at least in part by effects of the organic polyol molecule(s) interacting with the nitrite and the protons at the time of the acidification reaction, implying that mobility of the reactant molecules to orientate and reposition during the reaction under the influence of the polyol molecules may be important. Even if a polyol is not necessarily present, such as in the eighth aspect of the invention, it may be surmised that the same mobility between the reactants in the reaction of the one or more nitrite salt with the proton source may be important.

The organic carboxylic acid may, for example, be selected from salicylic acid, acetyl salicylic acid, acetic acid, citric acid, glycolic acid, mandelic acid, tartaric acid, lactic acid, maleic acid, malic acid, benzoic acid, formic acid, propionic acid, α-hydroxypropanoic acid, β-hydroxypropanoic acid, β-hydroxybutyric acid, β-hydroxy-β-butyric acid, naphthoic acid, oleic acid, palmitic acid, pamoic (embolic) acid, stearic acid, malonic acid, succinic acid, fumaric acid, glucoheptonic acid, glucuronic acid, lactobionic acid, cinnamic acid, pyruvic acid, orotic acid, glyceric acid, glycyrrhizic acid, sorbic acid, hyaluronic acid, alginic acid, oxalic acid, salts thereof, and combinations thereof. In particular embodiments, the organic carboxylic acid is selected from citric acid, salts thereof, and combinations thereof. In one particular embodiment, the organic carboxylic acid is citric acid or a salt thereof. The carboxylic acid may be or comprise a polymeric or polymerised carboxylic acid such as, for example, polyacrylic acid, polymethacrylic acid, a copolymer of acrylic acid and methacrylic acid, polylactic acid, polyglycolic acid, or a copolymer of lactic acid and glycolic acid. The term “organic carboxylic acid” used herein covers also partial or full esters of organic carboxylic acids or partial or full salts thereof, provided that those can serve as a proton source in use according to the present invention.

It is preferred that the pH of the proton source immediately before contact between the one or more nitrite salt and the proton source is buffered to control the pH within a known range and to restrict the rate of increase in the pH as the nitrite salt is consumed. Please see the section below headed “pH Control; Optional Buffer Systems” for more details. In particular, it is envisaged that at least one organic carboxylic acid of the proton source may suitably be present with the conjugate base thereof. The acid and its conjugate base may suitably form a buffer in the aqueous carrier. The buffer may be selected so that a desired pH is maintained thereby as the NOx generating reaction proceeds, preferably a pH in the range of about 3 to 9, for example about 4 to 8, preferably for physiological contact or for contact with living cells and organisms in the range of about 5 to about 8. The conjugate base, where present, may be added separately, or may be generated in situ from the proton source by adjustment of the pH using an acid and/or base, preferably a mineral acid and/or a mineral base.

The initial pH of an aqueous solution of the proton source including any desired buffer before (for example, immediately before) other components of the NOx generating reaction mixture are added that will affect the pH, or the pH of the reaction mixture at the start of the reaction with the one or more nitrite salt, is suitably in the range of about 3 to 9, for example about 4 to 8, for example about 5 to 8. The expression “initial pH” used herein in connection with the proton source means the pH of an aqueous solution of the proton source including any desired buffer before (for example, immediately before) other components of the NOx generating reaction mixture (including some but not all components thereof) are added that will affect the pH. Dry powdered proton source materials or other precursors of an aqueous solution of the proton source will be used in the appropriate amounts that will result in an aqueous solution having the desired initial pH.

If the proton source component is desired to be stored in a gel or other carrier system, for example an aqueous carrier, e.g. as an aqueous gel or solution, it is preferred that the system containing the proton source is buffered to a suitable pH to prevent maintain the acidity and prevent degradation of the proton source during storage. A pH of about 3-6, for example about 3-5, is preferred. If desired, the pH can be raised by addition of a base shortly before use of the proton source component.

Some patients have an intolerance to citric acid, for example. Patients should be tested for possible intolerance to the acid before treatment, and the acid component selected accordingly.

In one embodiment, the proton source component or portions of it may be provided for use in the invention in dry form, optionally in particulate form such as a powder. If desired, the proton source component or portions of it may be encapsulated or microencapsulated, e.g. for the purpose of controlling or delaying the reaction between the one or more nitrite salt and the proton source. The encapsulated form may particularly be used when a proton source normally has a liquid or gel state at room temperature. The dry form and/or the encapsulation may assist the storage of the proton source, whether alone or in admixture with other components of the reaction to generate the nitric oxide according to the invention. Still further, the dry form and/or the encapsulation may assist the incorporation of the proton source component, whether alone or in admixture with other components of the reaction to generate the nitric oxide according to the invention, into small objects such as medical devices. Such objects include, for example, wound dressings, bandages, vascular and other stents, catheters, pacemakers, defibrillators, heart assist devices, artificial valves, electrodes, orthopaedic screws and pins, and other thin medical and/or implantable articles. Please see the section below headed “Optional Encapsulation (e.g. Microencapsulation) of Components” for more details.

If desired, the one or more organic carboxylic acid, optionally encapsulated or microencapsulated, can be present in the proton source component as a dry powder or crystals, or in association with a gel or other carrier system, for example an aqueous carrier, e.g. as an aqueous gel or solution thereof. A proton source component containing an organic carboxylic acid in dry or powder form may conveniently be made up into solution before use by addition of water. The molarity of the total proton source (including any organic non-carboxylic reducing acid present) in such a solution before (for example, immediately before) addition of any other components of the NOx generating reaction mixture, and in particular before (for example, immediately before) initiation of the reaction with the nitrite may be in the range of about 0.001 M to about 5 M. In some embodiments, the molarity of the total proton source in such a solution before (for example, immediately before) addition of any other components of the NOx generating reaction mixture, and in particular before (for example, immediately before) initiation of the reaction with the nitrite is in the range of about 0.01 M to about 2 M. In some embodiments, the molarity of the total proton source in such solution prior to initiation of the reaction with the nitrite is in the range of about 0.1 M to about 2 M. In more particular embodiments, the molarity of the total proton source in such a solution prior to initiation of the reaction with the nitrite is in the range of about 0.2 M to about 1.6 M. In embodiments, the molarity of the total proton source in such a solution prior to initiation of the reaction with the nitrite can be in the range of 0.8 to 1.2 M. For example, the molarity of the total proton source in such a solution prior to initiation of the reaction with the nitrite may be about 0.8 M, about 0.9 M, about 1.0 M, about 1.1 M, about 1.2 M, about 1.5 M or about 1.7 M.

The expressions “molarity of the total proton source”, “concentration of the total proton source” and the like, used herein, shall be understood as referring to the concentration of whichever organic carboxylic acid(s) and/or organic non-carboxylic acid(s) is or are used as the proton source according to the present invention at a pH at which the proton (H⁺) donor moiety or at least one of the proton (H⁺) donor moieties (where there is more than one) is predominantly protonated, namely more than 50% protonated on a molar basis. In other words, if before initiation of the NOx generating reaction the pH is adjusted to a higher pH, whereby the degree of protonation is reduced, the molarity or concentration of the total proton source is not to be considered as reduced accordingly.

It is to be noted that the act of combining two or more precursor solutions of the NOx generating reaction mixture will cause a dilution of the concentration of each solute or combination of solutes in each solution, as is well known to those skilled in the art. For example, the act of mixing equal volumes of two 1 M solutions of solutes A and B causes the concentration of A to change to 0.5 M and the concentration of B to change to 0.5 M. Unless otherwise stated or implied, the concentration of proton source described herein is its concentration in an initial solution before (for example, immediately before) addition of any other components of the NOx generating reaction mixture that are added as liquids, e.g. solutions. The actual concentration in the NOx generating reaction mixture can readily be derived knowing the components of the reaction mixture and how it was prepared.

A proton source component in dry or powder form may conveniently be made up into solution before use by addition of water.

If desired, the one or more organic carboxylic acid, whether in dry form or in a carrier liquid, can be present in admixture or solution with the one or more polyol or some of such polyols.

It is preferred that the nitrite component is not brought into reactive contact with the proton source until it is desired to generate the nitric oxide, optionally other oxides of nitrogen and/or optionally precursors thereof. For this reason, the proton source component or a portion of it is preferably held in a reservoir or container of the kit, apparatus or device. However, it may alternatively be possible for dry components of the one or more nitrite salt or nitrite component, the proton source and the one or more polyol to be held as a dry composition, e.g. a particulate mixture, and for the reaction to be initiated by the simple addition of water or another suitable solvent or liquid carrier.

Proton Sources Components Comprising One or More Organic Non-Carboxylic Reducing Acid

The above discussion of proton source components comprising or consisting of one or more organic carboxylic acid applies analogously to proton source components comprising or consisting of one or more organic non-carboxylic reducing acids. In this section, the organic non-carboxylic reducing acids will be exemplified in more detail.

The expression “organic non-carboxylic reducing acid” herein refers to any organic reducing acid which does not contain a —COOH group in the molecule. An organic non-carboxylic reducing acid may be straight-chain or branched. The non-carboxylic reducing acid may be saturated or unsaturated. The non-carboxylic reducing acid may be aliphatic or aromatic. The non-carboxylic reducing acid may be acyclic or cyclic. The non-carboxylic reducing acid may be vinylogous.

The one or more organic non-carboxylic reducing acid, or each of them if more than one, should preferably have a pKa₁ less than about 7, more preferably less than 7.0.

For the reason explained above, it is generally preferred that at least one of the one or more acid selected from organic carboxylic acids and organic non-carboxylic reducing acids is not covalently attached to a polymer molecule, for example a polymer molecule forming a three-dimensional polymeric matrix of the hydrogel.

The organic non-carboxylic reducing acid may, for example, be selected from ascorbic acid; ascorbate palmitic acid (ascorbyl palmitate); ascorbate derivatives such as 3-O-ethyl ascorbic acid, other 3-alkyl ascorbic acids, 6-O-octanoyl ascorbic acid, 6-O-dodecanoyl ascorbic acid, 6-O-tetradecanoyl ascorbic acid, 6-O-octadecanoyl ascorbic acid and 6-O-dodecanedioyl ascorbic acid; acidic reductones such as reductic acid; erythorbic acid; oxalic acid; salts thereof; and combinations thereof. In one particular embodiment, the organic non-carboxylic reducing acid is ascorbic acid or a salt thereof.

The organic non-carboxylic reducing acid may carry one or more substituents, for example one or more hydroxyl group. Examples of hydroxyl-substituted organic non-carboxylic reducing acids which may be used in the present invention include the acidic reductones, for example reductic acid (2.3-dihydroxy-2-cyclopentanone).

It is preferred that the pH of the proton source and/or the reaction mixture after contact between the one or more nitrite salt and the proton source is buffered to control the pH within a known range and to control the increase in the pH as the nitrite salt is consumed. Please see the section below headed “pH Control; Optional Buffer Systems” for more details. In particular, it is envisaged that at least one organic non-carboxylic reducing acid of the proton source may suitably be present with the conjugate base thereof. The acid and its conjugate base may suitably form a buffer in the aqueous carrier. The buffer may be selected so that a desired pH is maintained thereby as the NOx generating reaction proceeds, preferably a pH in the range of about 3 to 9, for example about 4 to 8, preferably for physiological contact or for contact with living cells and organisms in the range of about 5 to about 8. The conjugate base, where present, may be added separately, or may be generated in situ from the proton source by adjustment of the pH using an acid and/or base, preferably a mineral acid and/or a mineral base.

The initial pH of an aqueous solution of the proton source including any desired buffer before (for example, immediately before) other components of the NOx generating reaction mixture are added that will affect the pH, or the pH of the reaction mixture at the start of the reaction with the one or more nitrite salt, is suitably in the range of about 3 to 9, for example about 4 to 8, for example about 5 to 8. Dry powdered proton source materials or other precursors of an aqueous solution of the proton source will be used in the appropriate amounts that will result in an aqueous solution having the desired initial pH.

If the proton source component is desired to be stored in a gel or other carrier system, for example an aqueous carrier, e.g. as an aqueous gel or solution, it is preferred that the system containing the proton source is buffered to a suitable pH to prevent maintain the acidity and prevent degradation of the proton source during storage. A pH of about 3-6, for example about 3-5, is preferred. If desired, the pH can be raised by addition of a base shortly before use of the proton source component.

Some reducing acids such as oxalic acid are toxic. The acid component should be selected accordingly.

One or more organic non-carboxylic reducing acid may be used in the proton source component in addition to, or in place of, the one or more organic carboxylic acid in the manner described above. Please see the section headed “Proton Sources Comprising One or More Organic Carboxylic Acid and Proton Source Component” for further details.

Organic Polyols and Organic Polyol Components

Aspects of the present invention involve one or more organic polyol. In the following the term “organic polyol component” or “polyol component” covers the organic polyol per se and any component of the reaction system for generating nitric oxide, optionally other oxides of nitrogen and/or optionally precursors thereof that contains the organic polyol.

The expression “organic polyol” herein refers to an organic molecule with two or more hydroxyl groups that is not a proton source, particularly for the nitrite salt reaction, and is not a saccharide or polysaccharide (the terms “saccharide” and “polysaccharide” include oligosaccharide, glycan and glycosaminoglycan). The organic polyol will thus have a pKa₁ of about 7 or greater, for example 7.0 or greater.

The expression “organic polyol” herein preferably excludes reductants. In one embodiment of the invention in all its aspects, therefore the organic polyol excludes reductants. Examples of reductants which are organic molecules with two or more hydroxyl groups and not a saccharide or polysaccharide are thioglycerol (for example, 1-thioglycerol), hydroquinone, butylated hydroquinone, ascorbic acid, ascorbate, erythorbic acid and erythorbate. Thioglycerol (for example, 1-thioglycerol), hydroquinone, butylated hydroquinone, ascorbate and erythorbate are thus preferably excluded from the expression “organic polyol” because they are reductants. Ascorbic acid and erythorbic acid are excluded from the expression anyway because they are proton sources, particularly for the nitrite salt reaction. For avoidance of doubt, we confirm that reductants which are proton sources, for example ascorbic acid and/or erythorbic acid, are not excluded from the proton sources of the invention or from the proton source components, combinations, kits, compositions, uses, methods or any other parts of the invention and its means of being put into practice in which they are present as proton sources.

The organic polyol may be cyclic or acyclic or may be a mixture of one or more cyclic organic polyol and one or more acyclic organic polyol. For example, the one or more organic polyol may be selected from one or more alkane substituted by two or more OH groups, one or more cycloalkane substituted by two or more OH groups, one or more cycloalkylalkane substituted by two or more OH groups, and any combination thereof. Most preferably the organic polyol does not carry any substituents other than OH.

Preferably the one or more organic polyol is one or more acyclic organic polyol. A preferred one or more acyclic organic polyol is selected from the sugar alcohols having 4, 5, 6, 7, 8, 9, 10, 11 or 12 carbon atoms. A preferred one or more acyclic organic polyol is selected from the alditols, for example the alditols having 4, 5, 6, 7, 8, 9, 10, 11 or 12 carbon atoms. It is preferred that the one or more organic polyol does not include a saponin, sapogenin, steroid or steroidal glycoside.

Alternatively the one or more organic polyol may be one or more cyclic organic polyol. In these embodiments, the one or more cyclic organic polyol may be a cyclic sugar alcohol or a cyclic alditol. For example the one or more cyclic polyol may be a cyclic sugar alcohol having 4, 5, 6, 7, 8, 9, 10, 11 or 12 carbon atoms or a cyclic alditol having 4, 5, 6, 7, 8, 9, 10, 11 or 12 carbon atoms. A specific example of a cyclic polyol is inositol.

In some embodiments the one or more organic polyol has 7 or more hydroxy groups. In particular embodiments the one or more organic polyol is a sugar alcohol or alditol having 7 or more hydroxy groups. In more particular embodiments the one or more organic polyol has 9 or more hydroxy groups. In further embodiments the one or more organic polyol is a sugar alcohol or alditol having 9 or more hydroxy groups. In some embodiments the one or more organic polyol has 20 or fewer hydroxyl groups. In particular embodiments the one or more organic polyol is a sugar alcohol or alditol having 20 or fewer hydroxy groups. In more particular embodiments the one or more organic polyol has 15 or fewer hydroxyl groups. In further embodiments the one or more organic polyol a sugar alcohol or alditol having 15 or fewer hydroxyl groups. The one or more organic polyol may have a number of hydroxyl groups in the range of 7 to 20, more particularly in the range of 9 to 15. In certain embodiments the one or more organic polyol includes 9, 12, 15 or 18 hydroxy groups.

Preferably the one or more organic polyol is sugar alcohol compound comprising, for example consisting of, one or more monosaccharide units and one or more acyclic sugar alcohol units. The one or more organic polyol may be a sugar alcohol compound comprising, for example consisting of, a straight chain of one or more monosaccharide units and one or more acyclic sugar alcohol units or a branched chain of one or more monosaccharide units and one or more acyclic sugar alcohol units.

A monosaccharide unit as used herein refers to a monosaccharide covalently linked to at least one other unit (whether another monosaccharide unit or an acyclic sugar alcohol unit) in the compound. An acyclic sugar alcohol unit as used herein refers to an acyclic sugar alcohol linked covalently to least one other unit (whether a monosaccharide unit or another acyclic sugar alcohol unit) in the compound. The units in the compound may be linked through ether linkages. In some embodiments, one or more of the monosaccharide units are covalently linked to other units of the compound through a glycosidic bond. In particular embodiments, each of the monosaccharide units are covalently linked to other units of the compound through a glycosidic bond. In certain embodiments, the sugar alcohol compound is a glycoside with a monosaccharide or oligosaccharide glycone and an acyclic sugar alcohol aglycone.

Preferred acyclic sugar alcohol units are sugar alcohol units having 4, 5, 6, 7, 8, 9, 10, 11 or 12 carbon atoms. In particular embodiments the acyclic sugar alcohol unit is selected from the group consisting of units of erythritol, threitol, arabitol, xylitol, ribitol, mannitol, sorbitol, galactitol, fucitol, iditol and volemitol.

In particular embodiments one or more of the monosaccharide units are a C₅ or C₆ monosaccharide unit. In other words, one or more of the monosaccharide units are a pentose or hexose unit. In more particular embodiments, each monosaccharide unit is a C₅ or C₆ monosaccharide unit. In particular embodiments one or more of the sugar alcohol units is a C₅ or C₆ sugar alcohol unit. In more particular embodiments each sugar alcohol unit is a C₅ or C₆ sugar alcohol unit.

In certain embodiments the sugar alcohol compound comprises, for example consists of, n monosaccharide units and m acyclic sugar alcohol units, where n is a whole number and at least one, m is a whole number and at least one and (n+m) is no more than 10. In certain embodiments the sugar alcohol compound comprises, for example consists of, a chain of n monosaccharide units terminated with one acyclic sugar alcohol unit, where n is a whole number between one and nine. In these embodiments, the chain of monosaccharide units may be covalently linked by glycosidic bonds. In particular embodiments, each monosaccharide unit is covalently linked to another monosaccharide unit or the acyclic sugar alcohol unit by a glycosidic bond. In certain embodiments the sugar alcohol compound comprises, for example consists of, a chain of 1, 2 or 3 monosaccharide units terminated with one acyclic alcohol unit. 1, 2, 3 or each monosaccharide unit may be a C₅ or C₆ monosaccharide unit. The acyclic alcohol unit may be a C₅ or C₆ sugar alcohol unit. Examples of the sugar alcohol compound include but are not limited to: isomalt, maltitol and lactitol (n=1); maltotriitol (n=2); and maltotetraitol (n=3).

Such sugar alcohol compounds may be described as sugar alcohols derived from a disaccharide or an oligosaccharide. Oligosaccharide, as used herein, refers to a saccharide consisting of three to ten monosaccharide units. Sugar alcohols derived from disaccharides or oligosaccharides may be synthesised (e.g. by hydrogenation) from disaccharides, oligosaccharides or polysaccharides (e.g. from hydrolysis and hydrogenation), but are not limited to compounds synthesised from disaccharides, oligosaccharides or polysaccharides. For example, sugar alcohols derived from a disaccharide may be formed from the dehydration reaction of a monosaccharide and a sugar alcohol. The one or more organic polyol may be a sugar alcohol derived from a disaccharide, trisaccharide or tetrasaccharide. Examples of sugar alcohols derived from disaccharides include but are not limited to isomalt, maltitol and lactitol. An example of a sugar alcohol derived from a trisaccharide includes but is not limited to maltotriitol. An example of a sugar alcohol derived from a tetrasaccharide includes but is not limited to maltotetraitol.

As suitable organic polyols there may be mentioned any selected from erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and any combination thereof. Glycerol can be used, and when present is preferably in association with one or more other organic polyol, for example erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or any combination thereof.

Many organic polyols contain one or more chiral centre and thus exist in stereoisomeric forms. All stereoisomeric forms and optical isomers and isomer mixtures of the organic polyols are intended to be included within the scope of this invention and patent. In particular, the D and/or L forms of all chiral organic polyols and all mixtures thereof may be used.

Interestingly, it has been found that the effect of the use of polyols in the present invention is stereochemistry dependent. Therefore, the selection of the optical isomeric form or optical isomer mixture of the one or more organic polyol for use in the present invention may affect the outcome of the reaction between the nitrite salt and the proton source, at least in terms of the amount of NO generated.

For example, sorbitol is a stereoisomer of mannitol, differing from each other in the orientation of one hydroxyl group. As shown in Example 2D and 2E below (FIGS. 5 and 6), the effects of sorbitol and mannitol on the output of the reaction between the nitrite salt and the proton source differ in otherwise identical reaction systems.

In particular embodiments, the organic polyol is selected from the group of arabitol, xylitol, mannitol, sorbitol and any combination thereof. The arabitol may be D or L arabitol or a mixture thereof. The xylitol may be D or L xylitol or a mixture thereof. The sorbitol may be D or L sorbitol or a mixture thereof. The mannitol may be D or L mannitol or a mixture thereof.

In specific embodiments the one or more polyol is a sugar alcohol compound comprising, for example consisting of, one or more monosaccharide units and one or more acyclic sugar alcohol units (including sugar alcohols derived from a disaccharide or an oligosaccharide) as described herein when used in the systems, methods, combinations, kits and compositions described herein are for use in or for the treatment of a tuberculosis infection or an antimicrobial method for reducing the number of tuberculosis bacteria.

In one embodiment, the organic polyol component may be provided for use in the invention in dry form, optionally in particulate form such as a powder. If desired, the organic polyol may be encapsulated or microencapsulated, e.g. for the purpose of controlling or delaying the involvement of the polyol in the reaction between the one or more nitrite salt and the proton source. The encapsulated form may particularly be used when an organic polyol normally has a liquid or gel state at room temperature. The dry form and/or the encapsulation may assist the storage of the organic polyol component, whether alone or in admixture with other components of the reaction to generate the nitric oxide according to the invention. Still further, the dry form and/or the encapsulation may assist the incorporation of the organic polyol component, whether alone or in admixture with other components of the reaction to generate the nitric oxide according to the invention, into small objects such as medical devices. Such objects include, for example, wound dressings, bandages, vascular and other stents, catheters, pacemakers, defibrillators, heart assist devices, artificial valves, electrodes, orthopaedic screws and pins, and other thin medical and/or implantable articles. Please see the section below headed “Optional Encapsulation (e.g. Microencapsulation) of Components” for more details.

Alternatively, the organic polyol component may include a carrier medium, for example an aqueous carrier liquid or a gel carrier. If the organic polyol is a normally liquid at room temperature, it may be used as such without any additional carrier component, or may be used in admixture with one or more carrier additives, e.g. water.

If desired, the one or more organic polyol, optionally encapsulated or microencapsulated, can be present in the polyol component as a dry powder or crystals, or in association with a gel or other carrier system, for example an aqueous carrier, e.g. as an aqueous gel or solution thereof. A polyol component containing an organic polyol in dry or powder form may conveniently be made up into solution before use by addition of water. The molarity of the total one or more polyol in such a solution prior to initiation of the reaction with the nitrite can be any concentration up to the saturation limit of the or each polyol in the solution. For example, the molarity of the total one or more polyol may be in the range of about 0.001 M to about 5 M. In some embodiments, the molarity of the total one or more polyol in such a solution prior to initiation of the reaction with the nitrite is in the range of about 0.01 M to about 2 M. In some embodiments, the molarity of the total one or more polyol in such solution prior to initiation of the reaction with the nitrite is in the range of about 0.1 M to about 2 M. In more particular embodiments, the molarity of the total one or more polyol in such a solution prior to initiation of the reaction with the nitrite is in the range of about 0.2 M to about 1.6 M. In embodiments, the molarity of the total one or more polyol in such a solution prior to initiation of the reaction with the nitrite can be in the range of 0.8 to 1.2 M. For example, the molarity of the total one or more polyol in such a solution prior to initiation of the reaction with the nitrite may be about 0.8 M, about 0.9 M, about 1.0 M, about 1.1 M, about 1.2 M, about 1.5 M or about 1.7 M.

It is to be noted that the act of combining two or more precursor solutions of the NOx generating reaction mixture will cause a dilution of the concentration of each solute or combination of solutes in each solution, as is well known to those skilled in the art. For example, the act of mixing equal volumes of two 1 M solutions of solutes A and B causes the concentration of A to change to 0.5 M and the concentration of B to change to 0.5 M. Unless otherwise stated or implied, the concentration of organic polyol described herein is its concentration in an initial solution before (for example, immediately before) addition of any other components of the NOx generating reaction mixture that are added as liquids, e.g. solutions. The actual concentration in the NOx generating reaction mixture can readily be derived knowing the components of the reaction mixture and how it was prepared.

A polyol component in dry or powder form may conveniently be made up into solution before use by addition of water.

If desired, the polyol, whether in dry form or in a carrier liquid, can be present in admixture or solution with the one or more nitrite salt or the proton source or some of such proton sources.

In particular embodiments in which the nitrite salt is kept separate, prior to use, from the other components of the reaction to generate the nitric oxide, the nitrite component may include the one or more polyol. In these embodiments, the organic carboxylic acid component may be substantially free of polyol. In alternative embodiments, the organic carboxylic acid component includes the one or more polyol. In these embodiments, the nitrite component may be substantially free of polyol. In further embodiments, the organic carboxylic acid component and the nitrite component may each include one or more polyols, which may be the same or different as between the two components.

In another embodiment, the organic carboxylic acid component and the nitrite component may be substantially free of polyol and one or more polyols may be included in a separate polyol component.

Relative Concentrations of Nitrite, Proton Source and Any Polyol in the Reaction Mixture

The total molar concentration of any one or more organic polyol in the polyol component or in the reaction solution at (or before) the start of the NOx generating reaction may suitably be between about 0.05 and about 3 times the total molar concentration of the nitrite ion, for example between about 0.1 and about 2, for example between about 0.25 and about 1.5, for example between about 0.3 and about 1.2 times the total molar concentration of the nitrite ion in the nitrite component or in the reaction solution. The same relative molar concentration between the one or more organic polyol and the nitrite ion is suitably provided in the components of the combination or kit according to the invention, or in the composition according to the invention, before (for example, immediately before) initiation of the NOx generating reaction.

The total molar concentration of any one or more organic polyol in the polyol component or in the reaction solution at (or before) the start of the NOx generating reaction may suitably be between about 0.05 and about 3 times the total molar concentration of the proton source, for example between about 0.1 and about 2 times the total molar concentration of the proton source in the proton source component or in the reaction solution. The same relative molar concentration between the one or more organic polyol and the proton source is suitably provided in the components of the combination or kit according to the invention, or in the composition according to the invention, before (for example, immediately before) initiation of the NOx generating reaction.

Optional Additional Components

The combinations, kits or compositions for use in the present invention may be incorporated in a range of diluents, carriers and excipients and/or provided in association with one or more additional components, particular functional components intended to provide one or more specific benefit to the combination, kit or composition in which it is used. Such diluents, carriers, excipients and/or additional components will generally be physiologically compatible when desired for use in vivo.

Examples of suitable physiologically compatible diluents, carriers and/or excipients include without limitation lactose, starch, dicalcium phosphate, magnesium stearate, sodium saccharin, talcum, cellulose, cellulose derivatives, sodium crosscarmellose, glucose, gelatin, sucrose, magnesium carbonate, magnesium chloride, magnesium sulfate, calcium chloride and the like.

Generally speaking, depending on the intended mode of administration the pharmaceutical formulation will contain about 0.005% to about 95%, preferably about 0.5% to about 50% by weight of the combination or composition of the present invention or components thereof. Actual methods of preparing such dosage forms are known, or will be apparent to those skilled in the art.

Excipients may be selected from known excipients depending on the intended use or administration route whereby the reactants and/or reaction products are to be delivered to the target site for the delivery of the nitric oxide, optionally other oxides of nitrogen and/or optionally precursors thereof. For example, creams, lotions and ointments may be formulated by incorporating the nitrite salt into excipients such as cream, lotion and ointment bases or other thickening agents and viscosifying agents (for example Eudragit L100, carbopol, carboxymethylcellulose or hydroxymethylcellulose). The proton source may be incorporated into excipients selected from carbopol, carboxymethylcellulose, hydroxymethylcellulose, methylcellulose, ethanol, lactose or in an aqueous base. If it is desired to form a film, film forming excipients such as, for example, propylene glycol, polyvinylpyrrolidone (povidone), gelatin, guar gum and shellac may be used.

Optional additional components may, for example, be selected from sweetening agents, taste-masking agents, thickening agents, viscosifying agents, wetting agents, lubricants, binders, film-forming agents, emulsifiers, solubilising agents, stabilising agents, colourants, odourants, salts, coating agents, antioxidants, pharmaceutically active agents and preservatives. Such components are well known in the art and a detailed discussion of them is not necessary for the skilled reader. Examples of auxiliary substances such as wetting agents, emulsifying agents, lubricants, binders, and solubilising agents include, for example, sodium phosphate, potassium phosphate, gum acacia, polyvinylpyrrolidone, cyclodextrrin derivatives, sorbitan monolaurate, triethanolamine acetate, triethanolamine oleate and the like. A sweetening agent or a taste-masking agent may, for example, include a sugar, saccharin, aspartame, sucralose, neotame or other compound that beneficially affects taste, after-taste, perceived unpleasant saltiness, sourness or bitterness, that reduces the tendency of an oral or inhaled formulation to irritate a recipient (e.g. by causing coughing or sore throat or other undesired side effect, such as may reduce the delivered dose or adversely affect patient compliance with a prescribed therapeutic regimen). Certain taste-masking agents may form complexes with one or more of the nitrite salts. Examples of thickening agents, viscosifying agents and film-forming agents have been given above.

The choice of pharmaceutically active agent and other additional components, for example those serving as diluents, carriers and excipients, may be determined by its suitability for the treatment regimen of the disease or medical condition concerned, as well as the desired administration route of the combination or composition according to the present invention. Reference can be made to standard reference works such as Martindale, 39^{th} Edition (2017), the Merck Index, 15^{th} Edition (2013), Goodman & Gilman's “The Pharmacological Basis of Therapeutics”, 13^{th} Edition (2017), the British National Formulary on-line (https://bnf.nice.org.uk/), Remington: “The Science & Practice of Pharmacy”, 22^{nd} Edition (2012), or the Physician's Desk Reference, 71^{st} Edition (2017).

Examples of administration routes by which the components and compositions according to the present invention may be administered to an animal (including human) subject for therapeutic purposes include topical (e.g. creams, lotions, gels, ointments, pastes, emollients, sprays), aural, nasal (e.g. sprays), vaginal, rectal (e.g. suppositories), oral (e.g. mists, sprays, mouthwashes, aerosols), enteral (e.g. tablets, pastilles, lozenges, capsules, linctuses, elixirs) and parenteral (e.g. injectable liquids), eye, ear, nose or throat (e.g. drops), or via the respiratory tract or lungs (e.g. mists, aerosols, powder inhalation).

Examples of pharmaceutically active agents that may be incorporated in the components and compositions or co-administered with the components and compositions according to the present invention include antibiotics, steroids, anaesthetics (for example topical anaesthetics such as lignocaine (lidocaine), amethocaine (tetracaine), xylocaine, bupivacaine, prilocaine, ropivfacaine, benzocaine, mepivocaine, cocaine or any combination thereof), analgesics, anti-inflammatory agents (for example non-steroidal anti-inflammatory drugs (NSAIDs)), anti-infective agents, vaccines, immunosuppressants, anticonvulsants, anti-dementia drugs, prostaglandins, antipyretics, anticycotics, anti-psoriasis agents, antiviral agents, vasodilators or vasoconstrictors, sunscreen preparations (e.g. PABA), antihistamines, hormones such as oestrogen, progesterone or androgens, antiseborrhetic agents, cardiovascular treatment agents such as alpha or beta blockers or Rogaine, vitamins, skin softeners, enzymes, mast cell stabilizers, scabicides, pediculicides, keratolytics, lubricants, narcotics, shampoos, anti-acne preparations, burn treatment preparations, cleansing agents, deodorants, depigmenting agents, diaper (nappy) rash treatment products, emollients, moisturizers, photosensitizing agents, poison ivy or poison oak or sumac products, sunburn treatment preparations, proteins, peptides, proteoglycans, nucleotides, oligonucleotides (such as DNA, RNA, etc), minerals, growth factors, tar-containing preparations, honey-containing preparations (for example, preparations containing Manuka honey), wart treatment preparations, wet dressings, wound care products, or any combination thereof.

Particular examples include analgesic agents, such as ibuprofen, indomethacin, diclofenac, acetylsalicylic acid, paracetamol, propranolol, metoprolol, and oxycodone; thyroid release hormone; sex hormones, such as oestragen, progesterone and testosterone; insulin; verapamil; vasopressin; hydrocortisone; scopolamine; nitroglycerine; isosorbide dintirate; anti-histamines, such as terfenadine; clonidine; nicotine; non-steroidal immunosuppressant drugs, such as cyclosporine, methotrexate, azathioprine, mycophenylate, cyclophosphamide, TNF-α antagonists and anti-IL5, -IL4Ra, -IL6, -IL13, -IL17, -IL23 cytokine monoclonal antibodies; anti-convulsants;

and drugs for Alzheimer's, dementia and/or Parkinson's disease, such as apomorphine and rivastigmine.

If desired, any of the optional additional components may be encapsulated or microencapsulated, e.g. for the purpose of controlling or delaying the release thereof. Please see the section below headed “Optional Encapsulation (e.g. Microencapsulation) of Components” for more details.

Optional Encapsulation (e.g. Microencapsulation) of Components

At least some of the components of the combinations, kits and compositions for use in the present invention may be encapsulated, for example microencapsulated.

The use of microencapsulated components for NO generation is useful because it provides for the prolonged production of a relatively unstable compound (such as NO) from precursors that are in a chemically stable form. Multiple microencapsulated reactants and/or one or more optional additional components can readily be stored mixed and in contact with one another in a dry environment, and the production of NO can be initiated simply by providing a small amount of water to the precursor mixture. Alternatively, such a mixture of microencapsulated reactants and/or one or more optional additional components can be applied directly to a subject, for example the skin, mucosal surface or into a subject's nose, mouth, respiratory tract and/or lungs, wherein the physiological environment itself provides sufficient water to cause release of therapeutic amounts of NO. A further advantage is that the volume occupied by the microencapsulated reactants and/or one or more optional additional components is relatively small, so that they can be readily incorporated into small objects such as medical devices. Such objects include, for example, wound dressings, bandages, vascular and other stents, catheters, pacemakers, defibrillators, heart assist devices, artificial valves, electrodes, orthopaedic screws and pins, and other thin medical and/or implantable articles.

One example of a production method for encapsulation or microencapsulation of a reactant and/or one or more optional additional components is spray-drying of a melt or polymer solution of the reactant and/or one or more optional additional components to produce a finely-divided powder of individual particles comprising the material dispersed within a polymer matrix. Other encapsulation or microencapsulation methods such as pan coating, air suspension coating, centrifugal extrusion, fibre spinning, fibre extrusion, nozzle vibration, ionotropic gelation, coacervation phase separation, interfacial cross-linking, in-situ polymerisation and matrix polymerisation may also be used. The encapsulation polymer is preferably biocompatible. Such polymers include ethyl cellulose, natural polymers such as zein (a prolamine seed storage protein found in certain grass species including maize and corn), chitosan, hyaluronic acid and alginic acid, or biodegradable polyesters, polyanhydrides, poly(ortho esters), polyphosphazenes, or polysaccharides (see Park et al, Molecules 10 (2005), pages 141-161). Compositions in which one chemical is microencapsulated as indicated above are well-known for delivery of pharmaceutical and other agents. See Shalaby and Jamiolkowski, U.S. Pat. No. 4,130,639; Buchholz and Meduski, U.S. Pat. No. 6,491,748. However, in virtually all of such compositions, it is the therapeutic agent that is microencapsulated, and the therapeutic agent is not produced by a reaction of microencapsulated reagents. Appropriate modification of the prior art teachings will, however, be within the skill of one of ordinary skill in the art. Nitric oxide releasing polymers have been described for medical articles that involve NO adducts/donors. See, e.g., Arnold, U.S. Pat. No. 7,829,553 (carbon-based diazeniumdiolates attached to hydrophobic polymers); Knapp, U.S. Pat. No. 7,135,189 (a nitrosothiol precursor and a nitric oxide donor).

pH Control; Optional Buffer Systems

The compositions may have a controlled pH value. In particular, the composition may have a pH value in the range of 3.0 to 8.0, or more particularly in the range 4.0 to 8.0. In more particular embodiments, the composition has a pH value in the range of 4.0 to 7.4. In yet more particular embodiments, the compositions may have a pH in the range of 4.0 to 6.0. In these embodiments, the compositions may have a pH in the range of 4.5 to 6.0.

The pH of the compositions may be controlled in any known manner. In particular embodiments, the pH of the organic carboxylic acid component or the organic reducing acid component is controlled prior to combination with the nitrite component. In some embodiments, the organic carboxylic acid component or the organic reducing acid component includes a buffer. The buffer may be pharmacologically acceptable buffer, such as a phosphate buffer.

In some embodiments, the buffer is formed by mixing the organic carboxylic acid or the organic non-carboxylic reducing acid and its salt counterpart. For example, the organic carboxylic acid component may comprise an organic carboxylic acid and a salt of the organic carboxylic acid. The organic non-carboxylic reducing acid component may include an organic non-carboxylic reducing acid and a salt of the organic non-carboxylic reducing acid. In particular embodiments, the organic carboxylic acid component includes citric acid and citrate. In other embodiments, the organic carboxylic acid component or the organic reducing acid component include ascorbic acid and ascorbate. In some embodiments the organic carboxylic acid component includes an organic carboxylic acid and a salt of a further organic acid. For example, the organic carboxylic acid component may include citric acid and ascorbate. In yet further embodiments, the organic carboxylic acid component may include an organic carboxylic acid, a salt of the organic carboxylic acid and a salt of a further organic carboxylic acid. For example, the organic carboxylic acid component may include citric acid, citrate and ascorbate.

In other embodiments, the buffer is formed by adjusting the pH of the organic carboxylic acid or the organic non-carboxylic reducing acid so that the acid (protonated form) coexists in admixture with its salt counterpart. This is suitably achieved by adding a strong mineral base and optionally a strong mineral acid to the organic carboxylic acid or the organic non-carboxylic reducing acid in such amounts as to generate a buffer system in situ. Examples of suitable strong mineral bases include sodium hydroxide, lithium hydroxide, potassium hydroxide, rubidium hydroxide and cesium hydroxide. Examples of suitable strong mineral acids include hydrochloric acid, sulphuric acid, hydrobromic acid and nitric acid.

The buffer may include one or more physiological buffers, especially when the combination or composition according to the invention is to contact cells or animal (including human) skin, mucosae or other tissues, such as in the case of administration to the nose, mouth, respiratory tract or the lungs. Examples of suitable physiologically compatible buffers include Good's buffers, which buffer in the pH range of about 5 to about 9, for example 2-amino-2-methyl-1.3-propanediol, N-2-aminoethanesulfonic acid (ACES), N-(2-acetamido)-iminodiacetic acid (ADA), N-(1,1-dimethyl-2-hydroxyethyl)-3-amino-2-hydroxypropanesulfonic acid (AMPSO), N,N-bis(2-hydroxyethyl)-2-aminoethanesulfonic acid (BES), N,N-bis(2-hydroxyethyl)glycine (BICINE), 2-bis(2-hydroxyethyl)amino-2-(hydroxymethyl)-1,3-propanediol (BIS-TRIS), 1,3-bis[tris(hydroxymethyl)methylamino]-propane (BIS-TRIS Propane), N-cyclohexyl-2-aminoethanesulfonic acid (CHES), 3-(N,N-bis[2-hydroxyethyl]amino)-2-hydroxypropanesulfonic acid (DIPSO), 4-(2-hydroxyethyl)-1-piperazinepropanesulfonic acid (EPPS), diglycine, N-(2-hydroxyethyl)piperazine-N′-(4-butanesulfonic acid) (HEPBS), 4-(2-hydroxyethyl)-1-piperazineethanesulfonic acid (HEPES), 3-(N-morpholino)propanesulfonic acid (MOPS), 3-morpholino-2-hydroxypropanesulfonic acid (MOPSO), piperazine-N,N′-bis(2-ethanesulfonic acid) (PIPES), piperazine-1,4-bis(2-hydroxy-3-propanesulfonic acid), dehydrate (POPSO), sodium phosphate dibasic, sodium phosphate monobasic, potassium phosphate dibasic, potassium phosphate monobasic, [tris(hydroxymethyl)methylamino]propanesulfonic acid (TAPS), 2-hydroxy-3-[tris(hydroxymethyl)methylamino]-1-propanesulfonic acid (TAPSO), 2-[(2-hydroxy-1,1-bis(hydroxymethyl)ethyl)amino]ethanesulfonic acid (TES), N-[tri(hydroxymethyl)-methyl]glycine (Tricine), or 2-amino-2-(hydroxymethyl)-1,3-propanediol (TRIZMA).

### Osmolarity of the Compositions

The solute strength of any solutions of the nitrite salt, the proton source, the organic polyol or any combinations thereof to be delivered to a physiological system, particularly by a route that will give rise to contact with the skin, mucosae, nose, mouth, respiratory tract or lungs of a human or animal subject should be controlled to avoid any undesirable dehydration of the subject's organs and tissues.

The osmolality (Osm), defined as the number of moles of solute dissolved in one kilogram of solvent, may be represented as osmoles per kilogram (Osmol/kg). The osmolality of any solutions to be administered to a human or animal subject in accordance with the present invention should be generally in the range of about from 100 to about 5000 mOsmol/kg, for example from about 100, 200, 300, 400, 500, 600, 700, 800, 900 or 1000 to about 2000, 2250, 2500, 2750, 3000, 3250, 3500, 3750, 4000, 4250, 4500, 4750 or 5000 mOsmol/kg.

### Mixing of the Components to Initiate NOx Generation

We have found that the order in which the components of the NOx generating system are mixed in order to initiate the NOx generation can have an effect on the outcome of using the NOx thereby generated. Evidence of this effect is provided in Example 6 below.

In that Example, we demonstrate that the efficacy of a composition according to the present invention to kill the bacterium M. tuberculosis HN878 in THP-1 cells is different, according to whether—on the one hand—the nitrite salt, the proton source and the organic polyol components are first mixed in the desired proportions at a concentration higher than desired in the composition in the form in which it is to be used, and that concentrate is then diluted, suitably with water, to arrive at the composition to be used, or—on the other hand—the nitrite salt, the proton source and the organic polyol components are first mixed in the desired proportions at the desired concentration for the composition in the form in which it is to be used.

Furthermore, it is not predictable, which way of mixing the components will produce the better outcome in terms of the antimicrobial effect. While generally it seems that diluting a relatively concentrated pre-mix to arrive at the composition to be used may produce a better antimicrobial effect against M. tuberculosis HN878 in THP-1 cells, in some cases that produces an outcome that is not so good as the method in which the components are first mixed at the desired concentration for the use.

In one embodiment of the present invention, therefore, a method of preparing the NOx generating composition comprises mixing the nitrite salt, the proton source and the organic polyol components in desired proportions at a concentration higher than desired in the composition in the form in which it is to be used, to form a concentrate pre-mix, and subsequently diluting that concentrate pre-mix, suitably with water, to provide the composition to be used.

In another embodiment of the present invention, therefore, a method of preparing the NOx generating composition comprises mixing the nitrite salt, the proton source and the organic polyol components in desired proportions at the desired concentration for the composition in the form in which it is to be used, to provide the composition to be used.

### PREFERRED EMBODIMENTS

Preferred embodiments of the first to eighth aspects of the present invention are those wherein on or more of the following is present:
the one or more nitrite salt comprises (for example, includes or consists essentially of or consists only of) one or more alkali metal or alkaline earth metal nitrite salt, for example: sodium nitrite; potassium nitrite; or any combination thereof;
the proton source comprises (for example, includes or consists essentially of or consists only of) ascorbic acid or ascorbic acid/ascorbate buffer; citric acid or citric acid/citrate buffer; or any combination of two or more thereof;
the molecules of the said ascorbic acid or ascorbic acid/ascorbate buffer, citric acid or citric acid/citrate buffer, or any combination of two or more thereof, are not covalently bonded to a polymer or macromolecule;
the one or more organic polyol comprises (for example, includes or consists essentially of or consists only of) a straight-chain sugar alcohol or alditol having from 4 to 12 carbon atoms and from 4 to 12 OH groups per molecule; for example sorbitol; mannitol; arabitol; xylitol; or any combination of two or more thereof;
the one or more organic polyol is a sugar alcohol compound comprising, for example consisting of, a chain of 1, 2 or 3 monosaccharide units terminated with one acyclic alcohol unit, optionally where. 1, 2, 3 or each monosaccharide unit is a C₅ or C₆ monosaccharide unit and/or the acyclic alcohol unit is a C₅ or C₆ sugar alcohol unit; for example, isomalt, maltitol, lactitol, maltotriitol, maltotetraitol;
the total molar concentration of the one or more organic polyol in the polyol component or in the reaction solution at or before the start of the NOx generating reaction is between 0.05 and 3 times the total molar concentration of the nitrite ion in the nitrite component or in the reaction solution;
the total molar concentration of the one or more organic polyol in the polyol component or in the reaction solution at or before the start of the NOx generating reaction is between 0.05 and 3 times the total molar concentration of the proton source in the proton source component or in the reaction solution;
the pH of the proton source before, particularly immediately before, initiation of the NOx generating reaction is in the range 3.0 to 9.0 for applications which do not involve contact between the reaction mixture and cells or animal (including human) skin (including mucosae), organs or other tissue;
the pH of the proton source before, particularly immediately before, initiation of the NOx generating reaction is in the range 4.0 to 8.0 for applications which involve contact between the reaction mixture and cells or animal (including human) skin (including mucosae), organs or other tissue;
the pH of the proton source before, particularly immediately before, initiation of the NOx generating reaction is in the range 5.0 to 8.0 for applications which involve contact between the reaction mixture and the nose, mouth, respiratory tract or lungs of an animal (including human) subject;
the microbe targeted is selected from the microbes listed below in the section headed “Targets for Antimicrobial Use”, for example without limitation Influenza virus, SARS-CoV, SARS-CoV-2, Mycobacterium tuberculosis, Mycobacterium abscessus, Pseudomonas aeruginosa including antibiotic-resistant strains thereof.

Preferred embodiments of the ninth aspect of the present invention are those wherein one or more of the following is present:
the one or more nitrite salt comprises (for example, includes or consists essentially of or consists only of) one or more alkali metal or alkaline earth metal nitrite salt, for example: sodium nitrite; potassium nitrite; or any combination thereof;
the proton source comprises (for example, includes or consists essentially of or consists only of) ascorbic acid or ascorbic acid/ascorbate buffer; citric acid or citric acid/citrate buffer; or any combination of two or more thereof;
the molecules of the said ascorbic acid or ascorbic acid/ascorbate buffer, citric acid or citric acid/citrate buffer, or any combination of two or more thereof, are not covalently bonded to a polymer or macromolecule;
the one or more organic polyol comprises (for example, includes or consists essentially of or consists only of) a straight-chain sugar alcohol or alditol having from 4 to 12 carbon atoms and from 4 to 12 OH groups per molecule; for example sorbitol; mannitol; arabitol; xylitol; or any combination of two or more thereof;
the one or more organic polyol is a sugar alcohol compound comprising, for example consisting of, a chain of 1, 2 or 3 monosaccharide units terminated with one acyclic alcohol unit, optionally where. 1, 2, 3 or each monosaccharide unit is a C₅ or C₆ monosaccharide unit and/or the acyclic alcohol unit is a C₅ or C₆ sugar alcohol unit; for example, isomalt, maltitol, lactitol, maltotriitol, maltotetraitol;
the total molar concentration of the one or more organic polyol in the polyol component or in the reaction solution at or before the start of the NOx generating reaction is between 0.05 and 3 times the total molar concentration of the nitrite ion in the nitrite component or in the reaction solution;
the total molar concentration of the one or more organic polyol in the polyol component or in the reaction solution at or before the start of the NOx generating reaction is between 0.05 and 3 times the total molar concentration of the proton source in the proton source component or in the reaction solution;
the pH of the proton source before, particularly immediately before initiation of the NOx generating reaction is in the range 3.0 to 9.0 for applications which do not involve contact between the reaction mixture and cells or animal (including human) skin (including mucosae), organs or other tissue;
the pH of the proton source before, particularly immediately before, initiation of the NOx generating reaction is in the range 4.0 to 8.0 for applications which involve contact between the reaction mixture and cells or animal (including human) skin (including mucosae), organs or other tissue;
the pH of the proton source before, particularly immediately before, initiation of the NOx generating reaction is in the range 5.0 to 8.0 for applications which involve contact between the reaction mixture and the nose, mouth, respiratory tract or lungs of an animal (including human) subject;
the microbe targeted is selected from the microbes listed below in the section headed “Targets for Antimicrobial Use”, for example without limitation Influenza virus, SARS-CoV, SARS-CoV-2, Mycobacterium tuberculosis, Mycobacterium abscessus, Pseudomonas aeruginosa including antibiotic-resistant strains thereof.

### Combinations and Compositions

The NOx generating reaction may be initiated in a number of ways. They are generally characterised by bringing the one or more nitrite salt and the proton source into contact under conditions whereby the NOx generating reaction can start.

The reaction may be initiated by combining separate components of a combination. The combining may be achieved in vitro, and the resulting composition may then be administered to a subject or applied to any surface to be treated according to the invention. Alternatively, the evolved gas may be administered to a subject or applied to any surface to be treated according to the invention. Still further, both uses of the resulting composition may proceed, spaced timewise so that the composition is administered to a subject or applied to any surface to be treated after some evolution of gas has taken place.

The combining may be stepwise, with, for example, dry powder forms of the components being initially mixed and then mixed with water or another liquid carrier medium to initiate the reaction. Alternatively, dry powder forms of the components can be initially mixed individually with water or another liquid carrier medium, and the two or more liquids subsequently mixed to initiate the reaction.

Alternatively, at least some of the components of the NOx generating reaction according to the present invention may be present in admixture in a single composition, and the NOx generating reaction initiated on the composition. One possible way to initiate the NOx generating reaction may, for example, be to add a critical component or additive that initiates the reaction, for example water if the components of the composition are in dry or encapsulated form; or the proton source if the components of the composition lack the proton source.

A kit according to the invention typically comprises one or more component of a combination according to the invention or a composition according to the invention, under circumstances in which the NOx generating reaction is prevented from occurring. The parts of the kit are typically held in containers, which may be separate or adapted to facilitate the mixing that would be required to initiate the NOx generating reaction. The critical initiating component for initiating the NOx generating reaction, which needs to be introduced to the other necessary components by a user of the kit, may for example be one of the nitrite salt component, the proton source component or the polyol component, or may be an additional ingredient, typically a commonly available component such as water, which may be supplied by the user.

The parameters of the combinations and compositions defined and described in this patent typically include physical parameters such as pH, concentration and osmolality. Wherever possible, these are to be measured before initiation of the NOx generating reaction. The pH parameter, unless otherwise stated, refers to the pH of the proton source in deionised water at the concentration intended for initiation of the NOx generating reaction. The concentration of a solution, unless otherwise stated, refers to the concentration before mixing with other components to initiate the NOx generating reaction. Typically, when the nitrite salt and the organic carboxylic acid or organic reducing acid react on mixing to generate nitric oxide gas, it is not possible to easily measure such parameters while the NOx generating reaction is in progress.

Furthermore, it will be noted that the concentration of ingredients when in the reaction mixture will not necessarily correspond to their concentration in the parts of the combination before mixing. For example, assume that the composition for initiating the NOx generating reaction according to the present invention is formed from approximately equal volumes of a nitrite component and a proton source component added together as pre-made solutions. In that embodiment, the as-mixed reaction composition has a nitrite concentration half the concentration of the nitrite component and a proton source concentration of half the concentration of the proton source component.

The parts of the combination and the compositions may be in any suitable physical form according to the intended use of the system during or after the NOx generating reaction. For example, the parts of the combination and the compositions may each be in the form of a liquid, gel, or film, so that the NOx generating reaction mixture is similarly in the form of a liquid, gel or film. The liquid may be adapted to be able to be nebulized for inhalation into the respiratory tract or the lungs. The parts of the combination and the compositions may be in the form of a mouthwash or drink, if the NOx generating mixture is intended to be applied to the mouth or throat. Alternatively, the parts of the combination and the compositions may be in the form of an ointment, lotion, or cream, if the NOx generating reaction mixture is intended to be applied to the skin in topical administration.

### Multicomponent Systems, Kits and Dispensers

The multicomponent system described herein may include a nitrite component and a proton source component, optionally with a polyol component, as defined in accordance with the present invention and as described herein. The components in the multicomponent system are adapted to be brought into contact with each other and the reaction mixture and/or the evolved gas dispensed by means of suitable containers or reservoirs for holding the components before use and means for mixing the components, dispensing the reaction mixture and/or the evolved gas, and generally controlling the said mixing and dispensing. In one preferred embodiment, the reaction mixture can be dispensed in the form of a mist or aerosol of droplets entrained in an airstream.

The kits and dispensers of the present invention generally comprise at least some of the containers for holding the components before use, the at least one device or other means for mixing the components, dispensing the reaction mixture and/or the evolved gas, and generally controlling the said mixing and dispensing, as well as that or those component(s), if any, that are contained in the container(s) of the kit or dispenser before use. Instructions for use, or directions to where instructions for use may be found, for example on-line instructions for use, may suitably be present. Such kits and dispensers constitute a further aspect of the present invention

Kits of the present invention may be relatively simple collections of containers and means for mixing the components, dispensing the reaction mixture and/or the evolved gas, and generally controlling the said mixing and dispensing. Such kits may suitably be provided for research purposes or where a wide latitude of variation in the mixing and dispensing operation can be expected and tolerated.

Other kits of the present invention may be more sophisticated collections of one or more container comprising consumables (being the combination(s) and/or composition(s) required by the user to initiate the NOx generating reaction, optionally with water or other commonly available ingredient(s) to be supplied by the user) together with one or more dispenser of the present invention.

Dispensers of the present invention will generally be adapted for a repeated similar action of dispensing the reaction mixture, a carrier that comprises the reaction mixture, and/or the evolved gas. The dispensers may comprise pumps or propellant systems to carry the composition comprising the reaction mixture generating NOx or the evolved gases out of the dispenser and direct it to a target. Propellant systems may use a pressurised and/or liquefied gas, which for medical use will suitably be pharmaceutically acceptable or biocompatible, for example pressurised air or pressurised/liquefied butane. Alternatively, suction from the lungs of a user may be used to carry the composition comprising the reaction mixture generating NOx or the evolved gases out of the dispenser and direct it to a target. Dispensers for use in the present invention may suitably comprise an actuator device such as a manually operable trigger or button whereby a user can actuate the dispenser. Such dispensers may be adapted for professional, research, consumer or patient use, and be correspondingly adapted to facilitate the intended route whereby the target is treated.

A wide range of kits and dispenser apparatus is in principle known, which can be used or readily adapted for holding the components before use, mixing the components or facilitating said mixing, dispensing the composition comprising the reaction mixture and/or the evolved gas, and generally controlling the said mixing and dispensing or facilitating said control.

For example:
syringes, for example twin barrel dispensing syringes.
container systems, for example pump action containers, squeeze action containers or shake action containers, for example comprising two containers, to mix at least the nitrite component and the proton source component and to dispense the composition comprising the NOx generating reaction or the evolved gas. Such systems are described in US 2019/0134080, the disclosure of which is incorporated herein by reference.
apparatus for holding the components before use in aqueous solution, mixing the components, nebulizing the liquid reaction mixture and dispensing the same for inhalation into the lungs of a human, and for generally controlling the said mixing and dispensing. Examples include soft mist inhalers, jet nebulizers, ultrasonic wave nebulizers and vibrating mesh nebulizers. The selection of suitable nebulizers, droplet sizes, co-agents, packaging forms, etc for inhalation of a nebulized NOx generating reaction medium by acidification of nitrite salts is described in WO 03/032928 and WO 2009/086470, the disclosures of which are incorporated herein by reference.
the above apparatus can be arranged to nebulize a pre-mixed liquid reaction mixture after it has been loaded into the nebulizer and dispensing the same for inhalation into the lungs of a human, and for generally controlling the said mixing and dispensing.
apparatus for holding the components before use in aqueous solution, mixing the components, aerosolising the liquid reaction mixture and dispensing the same for inhalation into the lungs of a human, and for generally controlling the said mixing and dispensing. Examples include metered dose inhalers. The selection of suitable droplet sizes, co-agents, packaging forms, etc for inhalation of a nebulized NOx generating reaction medium by acidification of nitrite salts is described in WO 03/032928 and WO 2009/086470, the disclosures of which are incorporated herein by reference.
techniques and apparatus for spraying nitric oxide releasing solutions into the upper respiratory tract are described in U.S. Pat. No. 9,730,956, the disclosure of which is incorporated herein by reference.
apparatus for holding the components before use in dry powder form and dispensing the same for inhalation into the lungs of a human. Examples include dry powder inhalers (DPI), which may be formulated as a single dose capsule or as a multi-dose dry powder inhaler, either as a reservoir powder or multi-dose separate blisters. The selection of suitable powder particle sizes, co-agents, packaging forms, etc for inhalation of the dry powder combination for providing a reaction medium within the lung to generate NO in situ by acidification of nitrite salts is described in WO 2009/086470, the disclosure of which is incorporated herein by reference.
dispensers for holding the components before use in solution form, aerating them and dispensing the same as a foam for skin disinfectant use or to treat skin disorders is described in US Patent Application No. 2013/0200109, U.S. Pat. No. 7,066,356 and US Patent Application No. 2019/0134080, the disclosures of which are incorporated herein by reference;
a transdermal patch assembly for holding the components and dispensing them to the skin of a subject is described in WO 2014/188175, the disclosure of which is incorporated herein by reference.

Dosages of the combinations and compositions or the evolved gas of the present invention can vary between wide limits, depending on the disease, disorder or condition to be treated (in the case of a medical treatment) or the effect desired (in the case of a non-medical treatment), the severity of the treatment required, and the condition, age and health of the subject to be treated or, in the case of non-medical treatments the nature of the target to be treated. In the case of medical treatments, ultimately a physician will determine the appropriate dosages to be used. In the case of non-medical treatments, the skilled person will be able to research appropriate dosages and treatment methods by a review of relevant literature of by reasonable trials.

In some embodiments, the composition in which the NOx generating reaction is taking place, or the evolved gas therefrom, can be administered to a target location, for example a microbial cell, living tissue, organ, structure or subject, within 600 seconds after combining the nitrite component and the proton source component. In this way, the target location may be exposed to a large burst of nitric oxide.

In some embodiments, the composition in which the NOx generating reaction is taking place can be formed in situ at or in the vicinity of a target location, for example on, within, or in the vicinity of, a microbial cell, living tissue, organ, structure or subject, including inanimate surfaces and spaces. In these embodiments, the administration is effectively 0 seconds after combining the nitrite component and the proton source component. In other embodiments, the composition is administered to the target location or its vicinity in the range of more than 0 seconds and less than 600 seconds after combining the nitrite component and the proton source component. In more particular embodiments, the composition is administered in the range of 0 and 120 seconds. In yet further embodiments, the composition is administered in the range of 0 and 60 seconds.

In other embodiments, the composition in which the NOx generating reaction is taking place, or the evolved gas therefrom, can be administered to a target location or its vicinity, for example a microbial cell, living tissue, organ, structure or subject, more than 600 seconds, for example more than 2000 seconds, for example more than 4000 seconds, for example more than 8000 seconds, after combining the nitrite component and the proton source component. The target location, for example microbial cell, living tissue, organ, structure or subject, may in that case not necessarily be exposed to a large burst of nitric oxide, but may still experience beneficial properties, such as antimicrobial effects. In these embodiments, the composition in which the NOx generating reaction is taking place, or the evolved gas therefrom, may be administered up to 48 hours after combining the nitrite component and the proton source component. In particular embodiments, the composition, or the evolved gas therefrom, may be administered up to several weeks or months, for example up to about 6 months, or up to about 2 months, or up to about 1 month, of up to about 3 weeks, or up to about 2 weeks, or up to about 1 week, or up to about 3 days, or up to 24 hours after combining the nitrite component and the proton source component.

The composition in which the NOx generating reaction is taking place, or the evolved gas therefrom, can be administered more than 48 hours after the nitrite component the proton source component are combined if stored appropriately. For example, the composition may be stored in a hermetically sealed container, for example under vacuum. The storage in a hermetically sealed container is typically performed no more than 24 hours after combining the nitrite salt and organic carboxylic acid or organic reducing acid. The composition may be stored in a hermetically sealed container no more than 600 seconds after combining the nitrite component and the proton source component. In this way, a proportion of nitric oxide gas may be retained. If the NOx generating composition is stored at low temperatures, for example temperatures in the range of about −30° C. to about +10° C., for example in the range of about 1° C. to about 10° C., the rate of evolution of gas can be substantially slowed, making available very long storage times of the compositions.

In a particular embodiment, an aerosol dispenser may include a plurality of reservoirs, with a first reservoir containing a nitrite component in liquid form (e.g. aqueous solution) and a second reservoir containing a proton source component in liquid form (e.g. aqueous solution). In this embodiment, each component may suitably be mixed with propellant before, during or after the said nitrite and proton source components are mixed.

In another particular embodiment, the dispenser may be a single-barrel syringe which contains the composition of the present invention. The viscosity of the composition may be selected to be able to be dispensed from the syringe by manual action or by powered operation of the syringe. For example, the composition may be a liquid or a gel.

In another particular embodiment, the dispenser may be a multi-barrel syringe having a first barrel containing a nitrite component and a second barrel containing a proton source component. The viscosity of the components may be selected to be able to be dispensed from the syringe by manual action or by powered operation of the syringe. For example, each component may independently be a liquid or a gel.

### Other Reservoirs for the Components: Hydrogels

In some embodiments of the present invention, molecular reservoirs, for example hydrogels, may be used. Hydrogels are highly hydrated, normally cross-linked, three-dimensional polymeric (homopolymeric or copolymeric) or macromolecular networks which have the ability to imbibe and retain many times their dry weight of water, other aqueous liquids or other non-aqueous hydrophilic liquids. Imbibing of liquids is normally accompanied by swelling of the hydrogel. By suitable selection of the component chemical groups covalently bonded to the polymer or macromolecule, acidic hydrogels or hydrogels with other special chemical properties can be prepared.

Hydrogels which can serve as a proton source component in the present invention are known. Examples of such acidic—COOH group containing hydrogels are described, for example, in WO 2007/007115, WO 2008/087411, WO 2008/087408, WO 2014/188174 and WO 2014/188175 and in the documents referred to therein, the disclosures of all of which are incorporated herein by reference. Uses of such hydrogels in skin care using NOx generation, including transdermal delivery of pharmaceuticals in conjunction with NOx generation, are described particularly in WO 2014/188174 and WO 2014/188175. Specific examples of such hydrogels include homopolymers and copolymers of acrylic acid, methacrylic acid, 2-acrylamido-2-methylpropanesulfonic acid (ATBS, available from Vinati Organics Ltd) and salts thereof. Polymers formed from monomers which include or consist of (meth)acrylic acid will include pendant carboxylic acid groups for use as proton source in accordance with the present invention.

Thus, for example, a multi-component system can comprise first acidic hydrogel pad or layer component comprising the proton source component, optionally further containing the organic polyol, and the other component may be the nitrite component. The nitrite component may, for example, be a liquid medium containing dissolved nitrite salt. In this way, a surface of the hydrogel pad or layer may be contacted with the nitrite component to initiate the NOx generating reaction. Alternatively, the nitrite component may be a solid carrier, for example a pad or layer, containing the nitrite salt in a form whereby it is accessible to dissolve in the imbibed liquid of the hydrogel on contact between the solid carrier and the hydrogel.

Typically, the solid carrier pad or layer is permeable (fully permeable or at least semi-permeable) to the diffusion of nitric oxide. In this way, nitric oxide may diffuse to an area of treatment when the solid carrier pad or layer and hydrogel are combined to combine the nitrite component and the proton source component. The solid carrier pad or layer may, for example, be a mesh, non-woven bat, film, foam, alginate layer or a membrane.

In particular embodiments, the solid carrier layer is a mesh. A mesh may be a number of connected strands of solid, typically flexible, that form a lattice of holes or gaps through which certain substances pass. The mesh may be woven or non-woven. In some embodiments, the mesh is non-woven.

The solid carrier layer, e.g. mesh, may be made of a polymeric material. Examples of suitable polymeric material include but are not limited to viscose, polyamide, polyester, polypropylene or blends thereof. The polymeric material may be treated to, for example, to increase its hydrophilicity. In particular embodiments, the solid carrier layer is a polypropylene mesh.

In particular embodiments, the solid carrier is absorptive and the nitrite component is at least partially absorbed, imbibed or impregnated in the solid carrier. The absorbed, imbibed or impregnated nitrite component may be solid (dried) or may be in aqueous solution within the solid carrier.

In particular embodiments, the solid carrier comprises more than one layer, and the nitrite component is absorbed, imbibed or impregnated in at least one layer or is coated on at least an outer layer. For example, the solid carrier may include 2, 3, 4, 5, 6, 7, 8, 9, 10 or more layers, such as polypropylene mesh layers, absorbed, imbibed, impregnated or coated with one or more nitrite salt in dry and/or solution form.

An acidic hydrogel has a natural buffering capacity due to the large supply of interior protonated pendant acidic groups, from which 1-1±ions can migrate via the imbibed aqueous medium to maintain a relatively acidic pH at the surface of the hydrogel structure as the pendant acidic moieties at the surface become deprotonated during the NOx generating reaction.

Non-acidic (e.g. neutral or basic) hydrogels are also known, in which a nitrite component and/or a polyol component can be imbibed and contained for use in the present invention. The proton source component can be contacted with such hydrogels, by the proton source being provided in a liquid medium contacted with the hydrogel, and/or by the proton source being absorbed in, imbibed in, impregnated in or coated onto, a solid carrier. In such hydrogels, it may be provided that none of the nitrite component, the proton source component or the polyol component is covalently bonded to the polymeric or macromolecular network of the hydrogel; for example, all of the components necessary for the present invention—taking into account that the nitrite component and the proton source component must not react together until initiation of the NOx generating reaction is desired—may be imbibed in the hydrogel and contained in the aqueous medium within the hydrogel mass but not covalently bonded to the polymer or macromolecule of the hydrogel.

The thickness of a hydrogel pad or layer may be in the range of 0.5 to 2 mm. In some embodiments, the thickness of the hydrogel pad or layer is in the range of 1 to 2 mm. In particular embodiments, the thickness of the hydrogel pad or layer is in the range of 1.0 to 1.6 mm.

The features described above in relation to the proton source component to generally will apply equally to any acidic hydrogel serving as the proton source component. Thus, for example, the hydrogel may contain a buffer to maintain the pH of the hydrogel in the range of 4.0 to 9.0, or 5.0 to 8.0.

In some embodiments, the hydrogel may include a barrier layer. The barrier layer is typically a polymeric film, such as polyurethane film, and located on an exterior surface of the hydrogel. In use, the barrier layer is typically located on the opposite surface of the hydrogel to the, for example, skin of a subject in order to provide a barrier between the multicomponent system as combined and the atmosphere. The surface of the barrier film adjacent to the hydrogel typically has a larger surface area than the adjacent hydrogel surface. In this way, the barrier layer may extend beyond the periphery of the hydrogel. In these embodiments, the barrier layer may have an adhesive around its peripheral edge to, in use, adhere the hydrogel to, for example, a subject's skin.

In a particular embodiment, the present invention provides a two-component system comprising: a) one or more mesh imbibed, impregnated or coated with one or more nitrite salt, such as NaNO₂; and
b) a hydrogel comprising a proton source comprising one or more acid selected from organic carboxylic acids and organic non-carboxylic reducing acids, wherein component (a) is separate from component (b) and wherein one or more of components (a) and (b) further comprises one or more organic polyol;

characterised by one or more of the following:

(a) the one or more organic polyol is present in a reaction output enhancing amount;

(b) the proton source is not solely a hydrogel comprising pendant carboxylic acid groups covalently bonded to a three-dimensional polymeric matrix;

(c) the one or more organic polyol is not solely glycerol;

(d) the one or more organic polyol is not solely glycerol when one or more viscosity increasing agent is used;

(e) the one or more organic polyol is not solely glycerol when one or more plasticizer is used;

(f) the one or more organic polyol is not solely polyvinyl alcohol;

(g) the one or more organic polyol is not solely polyvinyl alcohol when one or more viscosity increasing agent is used;

(h) any one or more of (b) to (g) above, wherein the words “is not solely” are replaced by “does not comprise”;

(i) the one or more organic polyol is not solely propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol;

(j) the one or more organic polyol does not comprise propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol.

For avoidance of doubt, it is hereby confirmed that the embodiments and preferences for the characterising features (a) to (j) described above in relation to the aspects of the invention apply equally to this embodiment.

Such a system may be used, for example, by combining the components (a) and (b) to initiate the NOx generating reaction. Such a combination may then be used in therapy or other treatment of the human or animal body, for example by topical application. The uses may be as described in WO 2014/188174 and WO 2014/188175, or may be as described below. The system may also be employed in non-medical uses as described below. When used for topical medical applications in which the system contacts a subject's skin (including mucosae), the one or mesh may be skin-contacting layer(s).

Uses in Therapy or Surgery

Compositions in which the NOx generating reaction is proceeding according to the present invention, and the evolved gas therefrom, have many applications in therapy and surgery, including curative and/or prophylactic therapy, surgery to correct diseases and disorders and conditions, cosmetic surgery, reconstructive surgery, including human and veterinary medicine and surgery. Where a physical disfigurement or abnormality that is responsive to treatment with the compositions or the evolved gas therefrom causes or exacerbates anxiety, depression or another mental disease or disorder, the treatment, prevention or alleviation of the physical condition can correspondingly treat, prevent or alleviate the mental condition, whereby the uses of the present invention extend also into the mental health arena.

Many physiological effects of nitric oxide and nitric oxide generating compositions and medical treatments based thereon have been reported in the literature, and as a result many therapeutic treatments have been developed. The following non-exhaustive list is provided as illustration. The listed uses as well as others not listed are encompassed within the present invention and patent.
Dilation of blood vessels by nitric oxide to raise blood supply and/or lower blood pressure (see van Faassen et al., Med. Res. Rev. 2009 September; 29(5), pages 683-741);
The acute effects of an oral nitric oxide supplement to lower blood pressure, improve vascular compliance and restore epithelial function in patients with hypertension are described by Houston et al. in J. Clin. Hypertens. (Greenwich), Jul. 2014, 16(7), pages 524-529;
Protection by nitric oxide of tissues from damage due to low blood supply (see van Faassen et al., Med. Res. Rev. 2009 September; 29(5), pages 683-741);
Action of nitric oxide as a neurotransmitter in nitrergic neurons, for example nitrergic neurons active on smooth muscle, for example in the gastrointestinal tract and erectile tissue (see Toda et al., Pharmacol. Ther., 2005 May; 106(2), pages 233-266);
Inhibition by nitric oxide of vascular smooth muscle contraction and growth, platelet aggregation and leukocyte adhesion to the endothelium, assisting vessel homeostasis (see Dessey and Ferron, Current Medical Chemistry—Anti-inflammatory and Anti-allergy Agents in Medicinal Chemistry, 2004; 3(3), pages 207-216);
Action of nitric oxide to decrease heart contractility and heart rate (see Navin et al., J. Cardiovascular Pharmacology, 2002; 39(2), pages 298-309);
Critical neonatal care to promote capillary and pulmonary dilation, for example treatment of primary pulmonary hypertension in neonatal patients, and post-meconium aspiration (see Barrington et al., The Cochrane Database of Systematic Reviews, 2017; 1, CD000399 (https://www.ncbi.nlm.nih.gov/pubmed/17375630); also Chotigeat et al., J. Med. Assoc. Thai., 2007; 90(2), pages 266-271; also Hayward et al., Cardiovascular Research, 1999; 43(3), pages 628-638);
Prevention of vascular damage, endothelial dysfunction and vascular inflammation, neuropathy and non-healing ulcers, and reducing the consequent danger of requiring lower limb amputation, in diabetes patients (see nfb University Studies—“Nitric Oxide Holds Promise for Diabetes”, http://www.nfb.org/Images/nfb/Publications/vod/vod212/vodspr0613.htm);
Improvement of hypoxemia in acute lung injury, acute respiratory distress syndrome and severe pulmonary hypertension; treatment of reversible causes of hypoxemic respiratory distress (see Mark et al., N. Eng. J. Med., Dec. 22, 2005; 353(25), pages 2683-2695);
Administration of nitric oxide as salvage therapy in patients with acute right ventricular failure secondary to pulmonary embolism (see Summerfield et al., 2011; Respir. Care 57(3), pages 444-448);
Treatment of angina, the effects of paraquat poisoning and other cardiovascular disorders (see Abrams, The American Journal of Cardiology, 1996; 77(13), pages 31C-37C;
Treatment of bladder contractile dysfunctions (see Moro et al., Eur. J. Pharmacol., January 2012; 674(2-3), pages 445-449; also Andersson et al., Br. J. Pharmacol. February 2008; 153(7), pages 1438-1444);
Treatment of acute and chronic lung infections and sepsis (see Fang et al., Nature Reviews. Microbiology, October 2004; 2(10), pages 820-832; also Goldfarb et al., Critical Care Medicine, January 2007; 35(1), pages 290-292);
Toxic reactive nitrogen intermediates (RNIB) including nitric oxide have been suggested as effector molecules in the antimycobacterial effect of activated murine macrophages against virulent Mycobacterium tuberculosis (see Chan et al., J. Exp. Med., April 1992; 175, pages 1111-1122);
Gaseous nitric oxide may be efficacious for the treatment of antibiotic resistant bacterial and fungal lung infections in patients with cystic fibrosis (see Deppisch et al., 9 Feb. 2016; “Gaseous nitric oxide to treat antibiotic resistant bacterial and fungal lung infections in patients with cystic fibrosis: a Phase I clinical study”, Springer, DOI 10.1007/s15010-016-0879-x);
Nitric oxide has been reported as a potential topical broad-spectrum antimicrobial agent for dermatologic diseases, with a small likelihood of resistance developing (see B L Adler and A J Friedman, Future Sci. OA, 2015; 1(1), F5037);
Nitric oxide is a neurotransmitter and has been associated with neuronal activity and various functions ranging from avoidance learning to genital erection in males and females (see Kim et al., J. Nutrition, 2004, 134, page 28735);
The use of nitric oxide to treat male impotence and erectile dysfunction is described in Sullivan et al., Cardiovascular Research, August 1999, 43(3), pages 658-665;
The potential uses of nitric oxide as a surgical adjuvant for assisting wound healing, reducing ischemia-reperfusion injury, assisting heart and lung recovery from surgery and assisting recovery from vascular surgery, as well as assisting postoperative recovery from orthopaedic surgery have been reported (see A Krausz and A J Friedman, Future Sci. OA, 2015; 1(1), FSO56);
The antimicrobial and wound healing effects of NO are described in WO 95/22335 and by Hardwick, et al., 2001, Clin, Sci. 100, pages 395-400;
European Patent No. 1411908 (Aberdeen University) reports data that are said to show that nitric oxide is effective to treat subungual infections, including Aspergillus niger;
Topical application of NOx generating compositions to the skin for treatment of fungal skin infections such as Tinea Pedis (Athlete's Foot) (see Weller, et al. J. Am. Acad. Dermatol., 1998 April, 38(4), pages 559-563);
Topical application of NOx generating compositions to the skin for treatment of viral skin infections (see WO 99/44622);
Topical application of NOx generating compositions to the skin for treatment of conditions where vasoconstriction is the underlying problem, such as Raynaud syndrome (also known as Raynaud's phenomenon) (see Tucker, et al. Lancet, 13 Nov. 1999, 354, 9191, pages 1670-1675);
The use of acidified nitrite as an agent to produce local production of nitric oxide at the skin surface for the treatment of peripheral ischaemia and associated conditions such as Raynaud's phenomenon and wounds such as post-operative wounds and burns is described in WO 2000/053193;
The use of a liquid nitric oxide releasing solution (NORS) to treat wounds in humans is claimed by U.S. Pat. No. 9,730,956 (Stenzler, et al.). The NORS is also alleged to have antibacterial, antifungal and/or antiviral properties, and data is provided which is said to demonstrate antibacterial efficacy on Acetobacter baumanii, methicillin-resistant Staphylococcus aureus, Escherichia coli and Mannheimia haemolytica. Data is provided which is said to demonstrate antiviral efficacy of the NORS on H1N1 influenza virus, Infectious Bovine Rhinotracheitis virus, Bovine Respiratory Syncytial virus and Bovine Parainfluenza-3 virus. Data is provided which is said to demonstrate antifungal efficacy of the NORS against Trichophyton rubrum and Trichophyton mentagrophytes;
Chou S-H, et al., The effects of debanding on the lung expression of ET-1, eNOS, and cGMP in rats with left ventricular pressure overload. Exp. Biol. Med. 2005, 231, pages 954-959;
Gladwin M T, et al., Nitrite as a vascular endocrine nitric oxide reservoir that contributes to hypoxic signaling, cytoprotection, and vasodilation, Am. J. Physiol. Heart Circ. Physiol. 2006, 291, pages H2026-H2035;
Hunter C J, et al., Inhaled nebulized nitrite is a hypoxia-sensitive NO-dependent selective pulmonary vasodilator. Nat. Med. 2004, 10, pages 1122-1127;
Ozaki M, et al., Reduced hypoxic pulmonary vascular remodeling by nitric oxide from the endothelium. Hypertension. 2001, 37, pages 322-327;
Rubin L J, 2006. Pulmonary arterial hypertension. Proc. Am. Thorac. Soc. 3, pages 111-115;
Yellon D. M. et al., 2007. Myocardial Reperfusion Injury, N. Engl. J. Med., 357, pages 1121-35;
Duranski M. R., et al., Cytoprotective effects of nitrite during in vivo ischemia-reperfusion of the heart and liver. J. Clin. Invest. 2005, 115, pages 1232-1240;
Jung K-H., et al., Early intravenous infusion of sodium nitrite protects brain against in vivo ischemic-reperfusion injury, Stroke, 2006, 37, pages 2744-2750;
Esme H., et al., Beneficial Effects of Supplemental Nitric Oxide Donor Given during Reperfusion Period in Reperfusion-Induced Lung Injury. Thorac. Cardiovasc. Surg. 2006, 54, pages 477-483;
The use of acidified nitrite for releasing NO as an agent to improve skin quality in humans is described in Chinese Patent Application No. CN 101028229;
The use of acidified nitrite or releasing NO as an agent to promote hair growth and prevent or treat alopecia in humans is described in Chinese Patent Application No. CN 101062050.

Other general discussions of the physiological effects of nitric oxide can be found, for example, in Lancaster et al., Proc Natl Acad Sci, 1996, 91, pages 8137-8141; Ignarro et al., Proc Natl Acad Sci, 1987, 84, pages 9265-9269; reviewed in Brent, J Cell Science, 2003, 116, pages 9-15; reviewed in Murad, N Engl J Med, 2006, 355, pages 2003-2011.

Pharmacological forms which have been published for delivery of NO are reviewed in Butler and Feelisch, Circulation, 2008, 117, pages 2151-2159.

The disclosure of each of the publications cited above is incorporated herein by reference.

The present invention is applicable to all therapeutic and surgical uses of nitric oxide and nitric oxide generating systems, including without limitation the specific therapies and surgical uses published in the above references and all other published therapies and surgical uses as well as therapies and surgical uses based on the underlying knowledge of the physiological effects of nitric oxide and the products of the nitric oxide generating reaction.

Vasodilation

The property of nitric oxide to induce vasodilation characterises many of the treatments using the combinations and compositions of the present invention and the gas evolved therefrom.

Particular examples of diseases, disorders and conditions responsive to vasodilation include, but are not limited to conditions associated with ischaemia and skin lesions.

Conditions associated with tissue ischaemia include Raynauld syndrome, severe primary vasospasm, and tissue ischaemia, for example tissue ischaemia caused by surgery, septic shock, irradiation or a peripheral vascular disease (for example diabetes and other chronic systemic disease).

When used in the treatment or prevention of conditions associated with tissue ischaemia as a result of surgery, a combination or composition of the present invention, or nitric oxide evolved from an NOx generating reaction using the present invention may be administered to a subject before, during or after the surgery. The combination, composition or evolved gas may be administered to the site of the surgery or in the vicinity of the site of the surgery. Examples of surgical procedures in which this treatment or prevention of tissue ischaemia may be used include transplantation surgery, tissue or organ grafting surgery, coronary surgery, carotid arterial catheterisation, surgery to provide indwelling arterial or venous catheters for administering systemic agents such as chemotherapy drugs, cosmetic surgery procedures including but not limited to a pedicled or rotation flap, repeat surgery where the incision is made at the same site as a prior surgical procedure, surgical operations performed in areas of poor skin and/or poor underlying tissue perfusion or where poor perfusion might be anticipated as a result of concomitant diseases (such as in patients with arteriosclerosis or diabetes mellitus), surgery in cases of trauma in which the blood vessels are damaged or compromised, and surgery to remove or rectify cutaneous or subcutaneous arteriovenous malformations.

For example, the combination, composition or evolved gas may be used in the treatment or prevention of ischemic reperfusion injury of an organ by administering a combination, composition or evolved gas according to the present invention to an organ. The organ may be one or more selected from the heart (e.g. to prevent or treat myocardial ischemia), the brain (e.g. to treat or prevent cerebral ischemia and or an infarction (stroke)), a lung (e.g. to treat or prevent ischemic reperfusion injury of the lung), a kidney (e.g. to treat or prevent ischemic reperfusion injury of the kidney), and a liver (e.g. to treat or prevent ischemic reperfusion injury of the liver). The surgery may be the transplantation of an organ. Administration of the combination, composition or evolved gas may follow an ischemic episode or may be prophylactic.

Transdermal Drug Delivery Uses

The property of nitric oxide to induce transdermal delivery of drugs represents another important utility of the combinations and compositions of the present invention and the gas evolved therefrom.

WO 02/17881 and WO 2014/188175, the disclosures of which are incorporated herein by reference, describe the use for transdermal drug delivery of combinations and compositions for generating nitric oxide and the gas evolved therefrom, and the same conditions, preferences and examples described in those publications for such uses are applicable also to the combinations and compositions of the present invention and the gas evolved therefrom.

Typically, the combinations and compositions of the present invention will comprise one or more pharmaceutically active agent to be transdermally delivered to a subject, and will be provided as a topical combination or composition form for application to the subject's skin. For examples of the pharmaceutically active agent(s) that can be used, please see the section headed “Optional Additional Components” above.

A suitable topical combination may comprise a nitrite-containing mesh and a separate proton-source-containing hydrogel, the two being adapted to be used together on the subject's skin as described above in the section headed “Other Reservoirs for the Compositions or Composition Systems; Hydrogels”. The polyol(s) and the pharmaceutically active agent(s) may be provided in one or more separate components of the combination or incorporated in the hydrogel, or any combination of these options may be employed respectively for the polyol(s) and for the pharmaceutically active agent(s).

Wounds, Skin Lesions and Burns Treatment

The properties of nitric oxide to induce vasodilation and the transdermal delivery of drugs and to kill or prevent the proliferation of microbes have given rise to another important utility of the combinations and compositions of the present invention and the gas evolved therefrom in the treatment of wounds, skin lesions and burns.

The conditions treatable using the present invention include ulcers, skin donor sites, surgical wounds (post-operative) burns (such as scalds, superficial, partial thickness and full thickness burns), lacerations and abrasions. Wounds may be chronic or acute. Ulcers may be of various origins, such as of arterial or venous origin. Examples of ulcers include leg ulcers, for example chronic leg ulcers or acute leg ulcers, pressure ulcers, for example chronic pressure ulcers or acute pressure ulcers, venous ulcers and ulcers associated with diabetes, such as diabetic foot ulcers.

WO 2014/188174, the disclosure of which is incorporated herein by reference, describes the use for treating wounds, skin lesions and burns of combinations and compositions for generating nitric oxide and the gas evolved therefrom, and the same conditions described in this publication is applicable also to the combinations and compositions of the present invention and the gas evolved therefrom.

Typically, the combinations and compositions of the present invention will comprise one or more pharmaceutically active agent, and will be provided as a topical combination or composition form for application to the subject's skin. For examples of the pharmaceutically active agent(s) that can be used, please see the section headed “Optional Additional Components” above. For the treatment of wounds, skin lesions and burns, the one or more pharmaceutically active agent may suitably be selected from analgesics and/or anaesthetics (for example, local anaesthetics) (for example, analgesics and/or anaesthetics to reduce chronic pain, acute pain or neuropathic pain), antimicrobial agents, disinfectants, anti-inflammatory agents and anti-scarring agents.

A suitable topical combination may comprise a nitrite-containing mesh and a separate proton-source-containing hydrogel, the two being adapted to be used together on the subject's skin as described above in the section headed “Other Reservoirs for the Compositions or Composition Systems; Hydrogels”. The polyol(s) and the pharmaceutically active agent(s) may be provided in one or more separate components of the combination or incorporated in the hydrogel, or any combination of these options may be employed respectively for the polyol(s) and for the pharmaceutically active agent(s).

Topical Antimicrobial Uses

In anti-microbial applications, the therapeutically-effective NO dose can be small, for example as low as a few hundred parts per million (ppm), for example 100 to 600 ppm (see, for example, Ghaffari et al., Nitric Oxide Biology and Chemistry, 2009, 14, pages 21-29, disclosure of which is incorporated herein by reference), but the effectiveness of the nitric oxide depends substantially on how long the skin contact is maintained (Ormerod et al., BMC Research Notes, 2011, 4, pages 458-465, the disclosure of which is incorporated herein by reference).

Proposals for slow topical release of nitric oxide have been published (see, for example, U.S. Pat. No. 6,103,275). However, the resultant topical NO dose lasts for less than one hour, which provides a poor topical antimicrobial action. As discussed above in the section headed “Multicomponent Systems, Kits and Dispensers”, and elsewhere, and as shown in the Examples below, the present invention enables much longer NO dosing periods, in both topical and non-topical administration systems, leading to substantial clinical advantages.

In particular, the combination and composition of the present invention have been found to enable the provision of a strong output of nitric oxide in the first approximately 200-500 seconds after the NOx generating reaction begins (“initial burst”), followed optionally by a long period of slower release of nitric oxide extending over many hours (“tail”) before the evolution of gas stops or falls below an effective level. The NO dose evolved by the combination and composition of the present invention exceeds the published minimum effective antimicrobial dose, leading to potential effective topical antimicrobial uses of the combination and composition of the present invention and the gas evolved therefrom.

The formulation of NOx generating combinations and compositions for topical antimicrobial application are well described in the prior art, for example US Patent Application No. 2014/0056957, the disclosure of which is incorporated herein by reference, and such formulations are applicable also to the combination and composition of the present invention. Another suitable topical combination may comprise a nitrite-containing mesh and a separate proton-source-containing hydrogel, the two being adapted to be used together on the subject's skin as described above in the section headed “Other Reservoirs for the Compositions or Composition Systems; Hydrogels”. The polyol(s) and any pharmaceutically active agent(s) may be provided in one or more separate components of the combination or incorporated in the hydrogel, or any combination of these options may be employed respectively for the polyol(s) and for the pharmaceutically active agent(s).

Other Dermal or Topical Treatments

Other topical applications of nitric oxide and nitric oxide generating compositions include stimulating hair growth and treating impotence and erectile dysfunction.

The combinations and compositions of the present invention may be formulated for topical application for such treatments.

Topical Dressings and Dressing Systems, for example Wound Dressings

In topical treatments, it is often desirable to cover or protect the treated area of skin while the treatment is being applied. This may assist in preventing contamination of a wound, assist in removing pus or debris from the healing process, prevent or restrict loss of the treatment composition on bathing or showering or through contact with clothing or as a result of a subject's normal activity, and cushion the treated area against knocks or rubbing.

For this purpose, it is common to incorporate the treatment in a topical dressing or dressing system, for example a wound dressing or dressing system. The dressing, or at least one component part of the dressing system, typically includes a backing sheet which may be water-impermeable or water-permeable and may optionally be provided with skin-adherent portions and optionally other layers such as gauze or pad layers.

In a further aspect, the present invention provides a topical dressing, for example a wound or skin dressing, or dressing system comprising a combination or composition according to the fifth aspect of the present invention, the dressing or at least one component of the dressing system comprising a backing sheet and optionally one or more other layer such as, for example, layers selected from gauze and pad layers. The combination or composition according to the fifth aspect of the present invention is suitably disposed on the skin-directed side of the backing sheet and arranged so that the desired skin area is treated with the NOx generating reaction mixture or the gas evolved therefrom when the dressing is applied to the skin and the NOx generating reaction initiated.

The dressing or dressing system may suitably be provided in a sealed sterile pack before use.

Nose, Mouth, Respiratory Tract and Lung Uses

The properties of nitric oxide to induce vasodilation and the transdermal delivery of drugs and to kill or prevent the proliferation of microbes have given rise to another important utility of the combinations and compositions of the present invention and the gas evolved therefrom in the treatment of the mucosae and tissues of the nose, mouth, respiratory tract and lungs, and/or the use of the nose, mouth, respiratory tract and lungs as the administration route for delivering to a human or animal subject the combinations and compositions of the present invention.

The conditions treatable using the present invention include lung diseases such as viral infections for example influenza, SARS-CoV or SARS-CoV-2, pulmonary arterial hypertension, ischemic reperfusion injury of the heart, brain and organs involved in transplantation, chronic obstructive pulmonary disease (COPD) (particularly, emphysema, chronic bronchitis), asthma including severe asthma and viral and bacterial induced exacerbations of asthma and refractory (non-reversible) asthma, intra-nasal or pulmonary bacterial infections such as pneumonia, tuberculosis, non-tuberculosis mycobacterial infections and other bacterial and viral lung infections, for example secondary bacterial infections following virus infections of the respiratory tract.

WO 2009/086470, the disclosure of which is incorporated herein by reference, describes the use for treating diseases of the nose, mouth, respiratory tract and lungs of nebulized liquid combinations and compositions for generating nitric oxide and the gas evolved therefrom, and/or the use of the nose, mouth, respiratory tract and lungs as the administration route for delivering such combinations and compositions to a human or animal subject, and the same conditions, preferences and examples described in that publication for such uses are applicable also to the combinations and compositions of the present invention and the gas evolved therefrom.

Typically, the combinations and compositions of the present invention for delivery to the nose, mouth, respiratory tract and lungs will comprise one or more pharmaceutically active agent. For examples of the pharmaceutically active agent(s) that can be used, please see the section headed “Optional Additional Components” above.

Two principle delivery methods are possible for performing the present invention via the delivery route of the nose, mouth, respiratory tract or lung(s). The first is that the combination or composition of the present invention is delivered directly to the nose, mouth, respiratory tract or lung(s). The second is that the gas evolved from the NOx generating reaction using the present invention is delivered to the nose, mouth, respiratory tract or lung(s) without the combination or composition of the present invention entering the patient's body.

1. Delivery of the Combination or Composition Directly to the Nose, Mouth, Respiratory Tract or Lung(s)

The combination or composition, or components thereof, may be delivered directly to the nose, mouth, respiratory tract or lung(s) in dry solid form, whereby the fluids of the mucosae dissolve the solid component materials and initiate the NOx generating reaction.

The components of the combination may be administered separately or together. In one preferred embodiment, the proton source or at least one component of it may be administered before the remaining components, so that a relatively acidic environment is established in the mucosae which assists a rapid initiation of the NOx generating reaction when the nitrite component contacts the proton source component in situ.

The delivery of any dry components of the combination, or the dry composition, directly to the nose, mouth, respiratory tract or lung(s) may suitably take place by dry powder inhalation using a dry powder inhaler, delivering to the subject a therapeutically effective dose of one or more dry powder component (e.g. one or more of the nitrite component, the proton source component and the polyol component), or the dry powder composition, wherein the dry powder inhaler delivers to the subject an aerosol containing particles of less than 6 microns volumetric mean diameter. The dry powder inhaler may be adapted for single or multiple dosing loaded with a dry powder so that the dry powder inhaler delivers between about 0.1 mg and about 100 mg per inhalation breath of one or more dry powder component, or the dry powder composition, to the subject in particles of less than 6 microns volumetric mean diameter.

Additionally, or alternatively, the combination or composition, or components thereof, may be delivered directly to the nose, mouth, respiratory tract or lung(s) in as a mist or spray of liquid droplets of a solution of one or more of the nitrite component, the proton source component and the polyol component.

The embodiments of the invention described herein are generally applicable to direct delivery to the nose, mouth, respiratory tract or lung(s) of the subject. Without limitation, for example, the combination or composition, or components thereof, may be administered directly to the nose, mouth, respiratory tract or lung(s) of the subject in association with one or more physiologically compatible diluents, carriers and/or excipients and/or provided in association with one or more additional components, particular functional components intended to provide one or more specific benefit. Examples of suitable physiologically compatible diluents, carriers and/or excipients include without limitation lactose, starch, dicalcium phosphate, magnesium stearate, sodium saccharin, talcum, cellulose, cellulose derivatives, sodium crosscarmellose, glucose, gelatin, sucrose, magnesium carbonate, magnesium chloride, magnesium sulfate, calcium chloride and the like. If desired, minor amounts of non-toxic auxiliary substances such as wetting agents, emulsifying agents, lubricants, binders, and solubilising agents, for example sodium phosphate, potassium phosphate, gum acacia, polyvinylpyrrolidone, cyclodextrrin derivatives, sorbitan monolaurate, triethanolamine acetate, triethanolamine oleate and the like may also be present. Generally speaking, depending on the intended mode of administration the pharmaceutical formulation will contain about 0.005% to about 95%, preferably about 0.5% to about 50% by weight of the combination or composition of the present invention or components thereof. Actual methods of preparing such dosage forms are known, or will be apparent to those skilled in the art. See, for example, Martindale, 39^{th} Edition (2017), the Merck Index, 15^{th} Edition (2013), Goodman & Gilman's “The Pharmacological Basis of Therapeutics”, 13^{th} Edition (2017), the British National Formulary on-line (https://bnf.nice.org.uk/), Remington: “The Science & Practice of Pharmacy”, 22^{nd} Edition (2012), or the Physician's Desk Reference, 71^{st} Edition (2017).

In one preferred embodiment, a combination or composition for delivery to the nose, mouth, respiratory tract or lung(s) of the subject will take the form of a unit dosage form such as a vial containing a liquid, solid to be suspended, dry powder, lyophilisate, or other composition, which combination or composition may suitably contain, along with the components of the NOx generating reaction, a diluent such as, for example, lactose, sucrose, dicalcium phosphate or the like; a lubricant such as magnesium stearate or the like; a binder such as starch, gum acacia, polyvinylpyrrolidone, gelatin, cellulose, cellulose derivatives or the like.

The delivery of any liquid droplets comprising components of the combination, or the composition in liquid droplet form, directly to the nose, mouth, respiratory tract or lung(s) may suitably take place by inhalation using a nebulizer, delivering to the subject a therapeutically effective dose of one or more liquid component (e.g. one or more of the nitrite component, the proton source component and the polyol component), or the composition in liquid form, wherein the nebulizer delivers to the subject an aerosol containing particles of less than 5 microns volumetric mean diameter. The nebulizer may be adapted for single or multiple dosing loaded with the liquid component of the combination or the liquid composition so that the nebulizer delivers between about 0.1 mg and about 100 mg per inhalation breath of one or more liquid component, or the composition in liquid form, to the subject in droplets of less than 5 microns volumetric mean diameter, preferably in droplets having a size in the range of about 2 to about 5 μm.

In one embodiment, a nebulizer is selected on the basis of allowing the formation of an aerosol of liquid droplets comprising components of the combination, or the composition in liquid droplet form having a mass median aerodynamic diameter (MMAD) predominantly between about 2 to about 5 microns.

In one embodiment, the delivered amount of liquid droplets comprising components of the combination, or the composition in liquid droplet form provides a therapeutic effect for pulmonary pathology, respiratory infections and/or extrapulmonary, systemic distribution to also treat extrapulmonary and systemic diseases.

Previously, two types of nebulizers, jet and ultrasonic, have been shown to be able to produce and deliver aerosol particles having sizes between 2 and 4 μm. These particle sizes have been shown as being optimal for middle airway deposition and hence, treatment of pulmonary bacterial infections caused by gram-negative bacteria such as Pseudomonas aeruginosa, Escherichia coli, Enterobacter species, Klebsiella pneumoniae, K. oxyloca, Proteus Pseudomonas aeruginosa, Serratia marcescens, Haemophilus influenzae, Burkholderia cepacia, Stenotrophomonas maltophilia, Alcaligenes xylosoxidans. Staphylococcus aureus and multidrug resistant Pseudomonas aeruginosa. However, unless a specially formulated solution is used, these nebulizers typically need larger volumes to administer sufficient amount of drug to obtain a therapeutic effect. A jet nebulizer utilizes air pressure breakage of an aqueous solution into aerosol droplets. An ultrasonic nebulizer utilizes shearing of the aqueous solution by a piezoelectric crystal. Typically, however, the jet nebulizers are only about 10% efficient under clinical conditions, while the ultrasonic nebulizer is only about 5% efficient. The amount of pharmaceutical deposited and absorbed in the lungs is thus a fraction of the 10% in spite of the large amounts of the drug placed in the nebulizer. Smaller particle sizes or slow inhalation rates permit deep lung deposition. Both middle-lung and alveolar deposition may be desired for this invention depending on the indication, e.g., middle airway deposition for antimicrobial activity, or middle and/or alveolar deposition for pulmonary arterial hypertension and systemic delivery. Exemplary disclosure of compositions and methods for formulation delivery using nebulizers can be found in, e.g., US 2006/0276483, including descriptions of techniques, protocols and characterization of aerosolized mist delivery using a vibrating mesh nebulizer. The disclosure of US 2006/0276483 is incorporated herein by reference.

Accordingly, in one embodiment, a vibrating mesh nebulizer is used to deliver in preferred embodiments an aerosol of the liquid droplets comprising components of the combination, or the composition in liquid droplet form. A vibrating mesh nebulizer comprises a liquid storage container in fluid contact with a diaphragm and inhalation and exhalation valves. In one embodiment, about 1 to about 5 ml of the liquid formulation to be delivered is placed in the storage container and the aerosol generator is engaged producing atomized aerosol of particle sizes selectively between about 1 and about 5 μm volumetric mean diameter.

Thus, for example, in preferred embodiments a nitrite component formulation or a proton source component, one or both of these optionally including one or more organic polyol according to the invention, is placed in a liquid nebulization inhaler and prepared in dosages to deliver from about 7 to about 700 mg from a dosing solution of about 1 to about 5 ml, preferably from about 17.5 to about 700 mg in about 1 to about 5 ml, more preferably from about 17.5 to about 350 mg in about 1 to about 5 ml, preferably about 0.1 to about 300 mg in about 1 to about 5 ml and more preferably about 0.25 to about 90 mg in about 1 to about 5 ml with volumetric mean diameter particles sizes between about 1 to about 5 μm being produced.

By non-limiting example, nebulized liquid comprising components of the combination, or the composition in liquid droplet form may be administered in the described respirable delivered dose in less than about 20 min, preferably less than about 10 min, more preferably less than about 7 min, more preferably less than about 5 min, more preferably less than about 3 min, and in some cases most preferable if less than about 2 min.

By non-limiting example, in other circumstances, a nebulized liquid comprising components of the combination, or the composition in liquid droplet form may achieve improved tolerability and/or exhibit an area-under-the-curve (AUC) shape-enhancing characteristic when administered over longer periods of time. Under these conditions, the described respirable delivered dose in more than about 2 min, preferably more than about 3 min, more preferably more than about 5 min, more preferably more than about 7 min, more preferably more than about 10 min, and in some cases most preferably from about 10 to about 20 min.

An example of separate component formulations may comprise (i) a nitrite salt in aqueous solution having a pH greater than about 6, for example in the range about 6 to about 8, for example about 7; and (ii) a proton source component in aqueous solution, at least the two separate liquid solution components (i) and (ii) being able to be admixed to form an NOx generating composition which may be used to load a nebulizer for delivery to a human patient or a veterinary subject.

For aqueous and other non-pressurized liquid systems, a variety of nebulizers (including small volume nebulizers) are available to aerosolize the components of the combination or the composition. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment, Inc.; Pari Respiratory, Inc. (Midlothian, VA); Mada Medical, Inc.; Puritan-Bennet; Schuco, DeVilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Heathcare, Inc. and DeVilbiss Health Care, Inc. Vibrating mesh nebulizers rely upon either piezoelectric or mechanical pulses to respirable liquid droplets generate. Other examples of nebulizers for use with nitrite, nitrite salt, or nitrite- or nitric oxide-donating compound described herein are described in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6.230.706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303 all of which are hereby incorporated by reference in their entireties.

Commercial examples of nebulizers that can be used with the liquid droplets comprising components of the combination, or the composition in liquid droplet form described herein include Respirgard II®, Aeroneb®, Aeroneb® Pro, AeroEclipse XL® and Aeroneb® Go produced by Aerogen (Aerogen, Inc., Galway, Ireland); AERx® and AERx Essence™ produced by Aradigm; Porta-Neb®, Freeway Freedom™, SideStream, SideStream Plus, Ventstream and I-neb produced by Respironics, Inc. (Murrysville, Pa., USA); and PART LC-Plus®, PARI LC-Star®, PARI LC-Sprint® and e-Flow™ produced by PARI, GmbH (PARI Respiratory Equipment, Inc., Midlothian, Va., USA; PARI GmbH, Starnberg, Germany). Any of these nebulizers can be used either with a face mask or mouth piece, according to manufacturer's specifications. By further non-limiting example, U.S. Pat. No. 6,196,219, is hereby incorporated by reference in its entirety.

In one embodiment, aqueous formulations containing soluble or nanoparticulate drug particles are provided. For aqueous aerosol formulations, the drug may be present at a concentration of about 0.67 mg/mL up to about 700 mg/mL; in certain preferred embodiments the nitrite salt is present at a concentration of from about 0.667 mg nitrite anion per ml to about 100 mg nitrite anion per ml. Such formulations provide effective delivery to appropriate areas of the lung, with the more concentrated aerosol formulations having the additional advantage of enabling large quantities of drug substance to be delivered to the lung in a very short period of time. In one embodiment, a formulation is optimized to provide a well-tolerated formulation. Accordingly, certain preferred embodiments comprise a nitrite salt (such as sodium nitrite, potassium nitrite or magnesium nitrite) and are formulated to have good taste, pH from about 4.7 to about 6.5, osmolarity from about 100 to about 3600 mOsmol/kg, and optionally in certain further embodiments, a permeant ion (e.g., chloride, bromide) concentration from about 30 to about 300 mM.

In one embodiment, the solution or diluent used for preparation of aerosol formulations has a pH range from about 4.5 to about 9.0, preferably from about 4.7 to about 6.5 (e.g., as an acidic admixture), or from about 7.0 to about 9.0 as a single vial configuration. This pH range improves tolerability, as does the inclusion of a taste-masking agent according to certain embodiments as described elsewhere herein. When the aerosol is either acidic or basic, it can cause bronchospasm and cough. Although the safe range of pH is relative and some patients may tolerate a mildly acidic aerosol, while others will experience bronchospasm. Any aerosol with a pH of less than about 4.5 typically induces bronchospasm. Aerosols with a pH from about 4.5 to about 5.5 will cause bronchospasm occasionally. Any aerosol having pH greater than about 8 may have low tolerability because body tissues are generally unable to buffer alkaline aerosols. Aerosols with controlled pH below about 4.5 and over about 8.0 typically result in lung irritation accompanied by severe bronchospasm cough and inflammatory reactions. For these reasons as well as for the avoidance of bronchospasm, cough or inflammation in patients, the optimum pH for the aerosol formulation was determined to be between about pH 5.5 to about pH 8.0.

Consequently, in one embodiment, aerosol formulations for use as described herein are adjusted to pH between about 4,5 and about 7.5 with the most preferred pH range for the acidic admixture from about 4.7 to about 6.5, and the most preferred pH range for the single vial configuration from about 7.0 to about 8.0. By way of non-limiting example, compositions may according to certain embodiments disclosed herein also include a pH buffer or a pH adjusting agent, typically a salt prepared from an organic acid or base, and in preferred embodiments an acidic excipient as described herein (e.g., a non-reducing acid such as citric acid or a citrate salt, such as sodium citrate) or a buffer such as citrate or other buffers described above and with reference to Table 1. These and other representative buffers thus may include organic acid salts of citric acid, ascorbic acid, gluconic acid, carbonic acid, tartaric acid, succinic acid, acetic acid, or phthalic acid, Tris, tromethamine, hydrochloride, or phosphate buffers.

Many patients have increased sensitivity to various chemical tastes, including bitter, salt, sweet, metallic sensations. To create well-tolerated drug products, taste masking may be accomplished through the addition of taste-masking agents and excipients, adjusted osmolality, and sweeteners.

Many patients have increased sensitivity to various chemical agents and have high incidence of bronchospastic, asthmatic or other coughing incidents. Their airways are particularly sensitive to hypotonic or hypertonic and acidic or alkaline conditions and to the presence of any permanent ion, such as chloride. Any imbalance in these conditions or a presence of chloride above a certain concentration value leads to bronchospastic or inflammatory events and/or cough which greatly impair treatment with inhalable formulations. Both of these conditions may prevent efficient delivery of aerosolized drugs into the endobronchial space, absent the advantageous uses of regulated pH, osmolality and taste-masking agent according to certain embodiments disclosed herein.

In some embodiments, the osmolality of aqueous solutions of the nitrite compound (or in distinct embodiments of the nitrite- or nitric oxide-donating compound) disclosed herein are adjusted by providing excipients. In some cases, a certain amount of a permeant ion, such as chloride, bromide or another anion, may promote successful and efficacious delivery of aerosolized nitrite salt. However, it has been discovered that for the nitrite components disclosed herein, the amounts of such permeant ions may be lower than the amounts that are typically used for aerosolized administration of other drug compounds.

Bronchospasm or cough reflexes may not in all cases be ameliorated by the use of a diluent for aerosolization having a given osmolality. However, these reflexes often can be sufficiently controlled and/or suppressed when the osmolality of the diluent is within a certain range. A preferred solution for aerosolization of therapeutic compounds which is safe and tolerated has a total osmolality from about 100 to about 3600 mOsmol/kg with a range of chloride concentration of from about 30 mM to about 300 mM and preferably from about 50 mM to about 150 mM, This osmolality controls bronchospasm, and the chloride concentration, as a permeant anion, controls cough. Because they are both permeant ions, bromide or iodide anions may be substituted for chloride. In addition, bicarbonate may be substituted for chloride ion.

Nanoparticulate drug dispersions can also be freeze-dried to obtain powders suitable for nasal or pulmonary delivery. Such powders may contain aggregated nanoparticulate drug particles having a surface modifier. Such aggregates may have sizes within a respirable range, e.g., about 2 to about 5 microns MMAD.

2. Delivery of the Gas Evolved from the NOx Generating Reaction to the Nose, Mouth, Respiratory Tract or Lung(s)

Inhalers for the delivery of metered amounts of nitric oxide to a patient's lungs are well known. Generally speaking, the nitric oxide is generated off-site and delivered to the hospital or clinic in pressurised cylinders which are connected to specialised delivery devices for use. The INOmax Therapy system may be mentioned as an example (BOC Healthcare, UK, https://www.bochealthcare.co.uk/en/products-and-services/products-and-services-by-category/medical-gases/inomax/inomax.html). The abbreviation INOmax (Inhaled Nitric Oxide) is generally used for the cylinders of the INOmax Therapy system and INOvent for the delivery devices. Evaluations of the INOmax Therapy system have been published, for example Kirmse, et al., Chest, lime 1998, 113(6), pages 1650-1657. The disclosure of this publication is incorporated herein by reference.

The method according to the first aspect of the present invention may suitably be performed in a dedicated NO manufacturing facility, and the gas product according to the second aspect of the present invention provided to users in pressurised cylinders in the normal manner. The pressurised gas cylinders are then used in association with distribution, monitoring, dosing, mixing and delivery apparatus in known manner.

Targets for Antimicrobial Uses

As previously described, the NOx generating reaction of the present invention, and the gas evolved therefrom, have a biocidal or biostatic effect on a potentially wide range of microorganisms, leading to many anti-microbial applications.

The microbes may, for example, be any one or more selected from bacterial cells, viral particles and/or fungal cells, or microparasites, and may be individual cells, organisms or colonies. Bacterial cells, viral particles and/or fungal cells or microparasites may be present on or in a host organism, for example as the gut microbiome of a human or other animal or in a bacterial infection of a human or other animal. The bacterial, and/or fungal cell and/or viral particle and/or microparasite may be in vitro, in vivo or ex vivo.

The present invention may be particularly useful in the treatment or prevention of microbial infections at the site of a skin lesion in a subject. The present invention may be particularly useful in the treatment of prevention of microbial infections in immunosuppressed subjects.

When the microbe is present in a bacterial infection, a fungal infection, viral or microparasitic infection of a human or other animal, the infection may, for example, be in the context of a disease such as the common cold, influenza, tuberculosis, SARS, COVID-19, pneumonia or measles.

1. Bacterial Cells

The bacterium may be a pathogenic bacterial species. The microbial infection may be an infection caused by a pathogenic bacterial species, including Gram positive and Gram negative, aerobic and anaerobic, antibiotic-sensitive and antibiotic-resistant bacteria.

Examples of bacterial species which may be targeted using the present invention include species of the Actinomyces, Bacillus, Bartonella, Bordetalla, Borrelia, Brucella, Campylobacter, Chlamydia, Chlamydophila, Clostridium, Corynebacterium, Enterococcus, Escherichia, Francisella, Haemophilus, Heliobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, or Yersinia genera. Any combination thereof can also be targeted by the present invention.

In particular embodiments, the microbe may be a pathogenic species of Corynebacterium, Mycobacterium, Streptococcus, Staphylococcus, Pseudomonas or any combination thereof.

In more particular embodiments, the microbe to be targeted can be selected from Actinomyces israelii, Bacillus anthracis, Bacteroides fragilis, Bordetella pertussis, Borrelia burgdorferi, Borrelia garinii; Borrelia afzelii; Borrelia recurrentis; Brucella abortus; Brucella canis; Brucella melitensis; Brucella suis; Campylobacter jejuni; Chlamydia pneumoniae; Chlamydia trachomatis; Chlamydophila psittaci; Clostridium botulinum; Clostridium difficile; Clostridium perfringens; Clostridium tetani; Corynebacterium diphtheria; Ehrlichia canis; Ehrlichia chaffeensis; Enterococcus faecalis; Enterococcus faecium; Escherichia coli, such as Enterotoxigenic E. coli (ETEC), Enteropathogenic E. coli, Enteroinvasive E. coli (EIEC), and Enterohemorrhagic (EHEC), including E. coli O157:H7; Francisella tularensis; Haemophilus influenza; Helicobacter pylori; Klebsiella pneumoniae; Legionella pneumophila; Leptospira species; Listeria monocytogenes; Mycobacterium leprae; Mycobacterium tuberculosis; Mycobacterium abscessus; Mycobacterium ulcerans; Mycoplasma pneumoniae; Neisseria gonorrhoeae; Neisseria meningitides; Pseudomonas aeruginosa; Nocardia asteroids; Rickettsia rickettsia; Salmonella typhi; Salmonella typhimurium; Shigella sonnei; Shigella dysenteriae; Staphylococcus aureus; Staphylococcus epidermidis; Staphylococcus saprophyticus; Streptococcus agalactiae; Streptococcus pneumoniae; Streptococcus pyogenes; Streptococcus viridans; Treponema pallidum subspecies pallidum; Vibrio cholera; Yersinia pestis; and any combination thereof.

In particular, the microbe may be selected from Chlamydia pneumoniae, Bacillus anthracis, Corynebacterium diphtheria, Haemophilus influenza, Mycobacterium leprae, Mycobacterium tuberculosis, Mycobacterium abscessus, Mycobacterium ulcerans, Pseudomonas aeruginosa, Staphylococcus aureus, Streptococcus pneumoniae, or any combination thereof.

The microbe may be an antibiotic-resistant or antibiotic-sensitive pathogenic bacterial species or an antibiotic-resistant or antibiotic-sensitive strain of a bacterial species. The use of nitric oxide to treat methicillin resistant Staphylococcus aureus (MRSA) and methicillin sensitive Staphylococcus aureus (MSSA) is described, for example, in WO 02/20026, the disclosure of which is incorporated herein by reference. An example of an antibiotic-resistant or antibiotic-sensitive pathogenic bacterial species which may be killed or treated using the present invention is thus methicillin resistant Staphylococcus aureus (MRSA) or methicillin sensitive Staphylococcus aureus (MSSA).

2. Fungal Cells

The microbe may be a pathogenic fungal species. The microbial infection may be an infection caused by a pathogenic fungal species, including pathogenic yeasts.

Examples of fungal species which may be targeted using the present invention include species of Aspergillus, Blastomyces, Candida (for example Candida auris), Coccidioides, Cryptococcus (in particular, Cryptococcus neofromans or Cryptococcus gattii), Hisoplamsa, Murcomycetes, Pneumocystis (for example Pneumocystis jirovecii), Sporothrix, Talaromyces, or any combination thereof.

Examples of fungal infections include aspergillosis (such as allergic bronchia pulmonary aspergillosis), Tinea pedis (athlete's foot), infections caused by a pathogenic species of Candida, such as vaginal yeast infections, fungal toenail infections and diaper rash, Tinea cruris (jock itch), and Tinea corporis (ringworm).

3. Virus Particles

The microbe may be a virus particle. The infection may be caused by a pathogenic virus.

Examples of viruses which may be targeted using the present invention include influenza viruses, parainfluenza viruses, adenoviruses, noroviruses, rotaviruses, rhinoviruses, coronaviruses, respiratory syncytial virus (RSV), astroviruses, and hepatic viruses. In particular, the compositions of the present invention may be used in the treatment or prevention of an infection caused by one of the group selected from H1N1 influenza virus, Infectious Bovine Rhinotracheitis virus, Bovine Respiratory Syncytial virus, Bovine Parainfluenza-3 virus, SARS-CoV, SARS-CoV-2, and any combination thereof.

In particular, the invention may be applied to treat of a disease or disorder caused by a viral infection. Examples of such diseases which may be targeted by the present invention include respiratory viral diseases, gastrointestinal viral diseases, exanthematous viral diseases, hepatic viral disease, cutaneous viral diseases, hemorrhagic viral diseases, and neurological viral diseases.

Respiratory viral infections include influenza, rhinovirus (i.e. common cold virus), respiratory syncytial virus, adenovirus, coronavirus infections, for example, COVID-19, and severe acute respiratory syndrome (SARS). Gastrointestinal viral diseases include norovirus infections, rotavirus infections, adenovirus infections and astrovirus infections. Exanthematous viral diseases include measles, rubella, chickenpox, shingles, roseola, smallpox, fifth disease and chikungunya virus disease. Hepatic viral diseases include hepatitis A, hepatitis B, hepatitis C, hepatitis D and hepatitis E. Cutaneous viral diseases include warts, such as genital warts, oral herpes, genital herpes and molluscum contagiosum. Hemorraghic viral diseases include Ebola, Lassa fever, denghue fever, yellow fever, Marbug hemorrhagic fever and Crimean-Congo hemorrhagic fever. Neurological viral diseases which may be targeted using the present invention include polio, viral meningitis, viral encephalitis and rabies.

4. Parasitic Microorganisms

The microbe may be a parasitic microorganism (microparasite). The infection may be cause by a pathogenic parasitic microorganism.

Examples of parasitic microorganisms which may be targeted using the present invention include protozoa.

In particular, the invention may target the protozoa groups of Sarcodina (e.g. amoeba, for example Entamoeba such as Entamoeba histolytica or Entamoeba dispar), Mastigophora (e.g. flagellates, for example Giardia and Leishmania), Ciliophora (e.g. ciliates, for example Balantidium), Sporozoa (e.g. Plasmodium and Cryptosporidium), and any combination thereof.

Parasitic infections that may be treated using the present invention include malaria, amoebic dysentery and leishmaniasis (e.g. cutaneous leishmaniasis, mucocutaneous leishmaniasis or visceral leishmaniasis).

Human/Animal Hosts or Subjects

The subject may be an animal or human subject. The term “animal” herein generally can include human; however, where the term “animal” appears in the phrase “an animal or human subject” or the like, it will be understood from the context to refer particularly to non-human animals or that the reference to “human” merely particularises the option that the animal may be a human to avoid doubt.

In particular embodiments, the subject is a human subject. The human subject may be an infant or adult subject.

In particular embodiments, the subject is a vertebrate animal subject. The vertebrate animal may be in the Class Agnatha (jawless fish), Class Chondrichthyes (cartilaginous fish), Class Osteichthyes (bony fish), Class Amphibia (amphibians), Class Reptilia (reptiles), Class Ayes (birds), or Class Mammalia (mammals). In particular embodiments, the subject is an animal subject in the Class Mammalia or Ayes.

In particular embodiments, the subject is a domestic species of animal. The domestic species of animal may be one of:
commensals, adapted to a human niche (e.g., dogs, cats, guinea pigs)
prey or farm animals sought or farmed for food (e.g., cows, sheep, pig, goats); and
animals for primarily draft purposes (e.g., horse, camel, donkey)

Examples of domestic animals include, but are not limited to: alpaca, addax, bison, camel, canary, capybara, cat, cattle (including Bali cattle), chicken, collared peccary, deer (including fallow deer, sika deer, thorold's deer, and white-tailed deer), dog, donkey, dove, duck, eland, elk, emu, ferret, gayal, goat, goose, guinea fowl, guinea pig, greater kudu, horse, llama, mink, moose, mouse, mule, muskox, ostrich, parrot, pig, pigeon, quail, rabbit, rat (including the greater cane rat), reindeer, scimitar oryx, sheep, turkey, water buffalo, yak and zebu.

Organs, Structures and Internal Spaces of Animal/Human Hosts or Subjects

The organ to which the compositions or the multicomponent systems of the present invention are administered are not limited. Examples of organs include the skin and organs of the respiratory system, the genitourinary system, the cardiovascular system, the digestive system, the endocrine system, the excretory system, the lymphatic system, the immune system, the integumentary system, the muscular system, the nervous system, the reproductive system, and the skeletal system.

Examples of organs of the cardiovascular system include the heart, lungs, blood and blood vessels. Examples of organs of the digestive system salivary glands, esophagus, stomach, liver, gallbladder, pancreas, intestines, colon, rectum and anus. Examples of organs of the endocrine system include the hypothalamus, pituitary gland, pineal body or pineal gland, thyroid, parathyroids and adrenals, i.e., adrenal glands. Examples of organs of the excretory system include kidneys, ureters, bladder and urethra. Examples of organs of the lymphatic system include the lymph and the nodes and vessels. Examples of organs of the immune system include tonsils, adenoids, thymus and spleen. Examples of organs of the integumentary system include skin, hair and nails of mammals, as well as scales of fish, reptiles, and birds, and feathers of birds. Examples of organs of the nervous system include brain, spinal cord and nerves. Examples of organs of the reproductive system include the sex organs, such as ovaries, fallopian tubes, uterus, vulva, vagina, testes, vas deferens, seminal vesicles, prostate and penis. Examples of the organs of the skeletal system include bones, cartilage, ligaments and tendons.

Cavities of the human subject include but are not limited to a mouth, nose, ear, throat, respiratory tract, lungs, gastrointestinal tract, dorsal body cavity, such as the cranial cavity or the vertebral cavity, or a ventral body cavity, such as the thoracic cavity, the abdominal cavity or the pelvic cavity.

In vitro Antimicrobial Treatments of Surfaces

The components and compositions of the present invention, and the evolved gas from the NOx generating reaction according to the present invention, may be used to apply antimicrobial treatments in vitro. By “in vitro” is meant that the surface being treated is not a living organism, even if it may be intended ultimately for a medical application.

Examples of such utility include methods for sterilising surgical instruments, hypodermic needles and other medical devices before use, as well as cleaning or treatment of surfaces, whether in a hospital or clinic or anywhere else, to reduce or prevent the spread of a pathogen.

Other examples include methods for sterilising prostheses and implantable devices such as stents (for example coronary stents), surgical screws, rods, plates and splints, orthopaedic implants, cardiac pacemakers, insulin infusion devices, catheters, ostomy appliances, intraocular lenses, cochlear implants, electrical pain-reducing implants, implantable contraceptive devices, neurostimulators, artificial heart valves, electrodes, intravenous drips and drug delivery devices, and the like before locating the device within a subject's body.

If desired, the components or compositions of the present invention may be coated onto the surface of the prosthesis or implantable device, whereby the NO evolved in the NOx generating reaction may perfuse to other tissues or organs or exert other physiological effects in the vicinity of the prosthesis or implanted device.

Techniques for biocompatibilising the surfaces of prostheses or implantable devices, including incorporation of functional coatings, such as coatings comprising the components or compositions of the present invention, are well known to those skilled in the art. See, for example, Gultepe et al., Advanced Drug Delivery Reviews, 8 Mar. 2010, 62(3), pages 305-315; and U.S. Pat. Nos. 5,702,754 and 6,270,788, and the publications referred to therein, the disclosure of all of which are incorporated herein by reference.

Compositions and methods for more general antimicrobial treatment of inanimate surfaces are well known in the art and do not require extensive description here. Antibacterial compositions are used, for example, in the health care industry, food service industry, meat processing industry and in the private sector by individual consumers. Antibacterial cleansing compositions typically contain one or more active antibacterial agent or components thereof, a surfactant, and one or more other ingredients, for example dyes, fragrances, pH adjusters, thickeners, skin conditioners and the like, in an aqueous and/or alcoholic carrier. Broad spectrum antiseptic or antimicrobial compositions aim to reduce the pathogen load of a range of pathogens on a surface. Typically the composition is a liquid (or is made up to be a liquid from a solid pre-mix prior to use), the liquid—after any desired adjustment of concentration, suitably by addition of water—being spread or sprayed onto a surface to be treated, often with the aid of a cloth or other wiping device, and may then be left to dry on or wiped off. The conventional compositions and methods of treatment of surfaces are in principle applicable to be used with the present invention, whereby the active antimicrobial agent is or comprises the NOx generating composition or the components thereof according to the present invention.

For further discussion and examples of known antimicrobial compositions and methods of use which may be used in association with the present invention, we refer for example to U.S. Pat. Nos. 6,110,908; 5,776,430; 5,635,462; 6,107,261; 6,034,133; 6,136,771; 8,034,844; European Patent Application No. EP 0505935; and PCT Patent Applications Nos. WO 98/01110; WO 95/32705; WO 95/09605; and WO 98/55096; the contents of which are incorporated herein by reference in their entirety.

Uses in Improving Wellbeing of Humans and/or Animals

In addition to the medical uses discussed above, the present invention may be used in non-therapeutic applications in human or animal subjects. A non-therapeutic application is distinguished from a therapeutic application in that the subject is healthy or the application does not target for treatment any diagnosed disease, disorder or condition which the subject does have.

Non-therapeutic applications may include treatments which aim to improve the well-being or the feeling of well-being of the subject, or to raise the metabolic efficiency or the immune system activity of the subject, so that the subject is better able to function normally or to fight off a future infection. Non-therapeutic applications also comprise treatments which assist the cognitive functions of the subject or engender feelings of confidence and control.

For use in such non-therapeutic applications the combinations and compositions of the present invention may be formulated analogously to pharmaceutical formulations or in non-pharmaceutical ways. For further details of formulations analogous to pharmaceutical formulations, please see the section above headed “Optional Additional Components”. Non-pharmaceutical formulations may suitably include food additives, nutraceutical formulations, foodstuffs, beverages and beverage additives. The formulations adapted to be added to foodstuffs and beverages may suitably be in the form of liquids or powders. Nutraceutical formulations may suitably be in the form of tablets, capsules or orally ingestible liquids.

As mentioned above in the section headed “Uses in therapy or surgery”, medical and/or surgical uses of the present invention may provide secondary benefits to a patient in terms of enhanced wellbeing or confidence.

Plant Uses

Beneficial effects of nitric oxide on live or dead plants are known. The present invention includes the application of the methods, apparatus, combinations, kits, compositions, uses and the gas evolved therefrom to providing beneficial effects to live or dead plants.

Examples of known uses of nitric oxide and nitric oxide generating systems on plants include the following:
Prevention or delay by nitric oxide of wilting of cut flowers and plants (see Siegel-Itzkovich, B M J, 1999; 319(7205), page 274; also Mur et al., 2013; “Nitric oxide in plants: an assessment of the current state of knowledge”, AoB PLANTS doi:10.1093/aobpla/p1s052 (https://doi.org/10.1093%2Faobpla%2Fpls052));
Regulation by nitric oxide of plant-pathogen interaction, promotion of the plant hypersensitive response, symbiosis with organisms in nitrogen-fixing root nodules, development of lateral and adventitious roots and root hairs, and control of stomatal opening (see Mur et al., 2013; cited above);
Role of nitric oxide in antioxidant and reactive oxygen species responses in plants (see Verma et al., 2013; “Nitric oxide (NO) counteracts cadmium-induced cytotoxic processes mediated by reactive oxygen species (ROS) in Brassica juncea: cross-talk between ROS, NO and antioxidant responses”; in BioMetals);
Role of nitric oxide in signalling pathways of auxin, cytokinin and other plant hormones (see Liu et al., Proceedings of the National Academy of Sciences, 2013; 110(4), pages 1548-1553).

The disclosure of each of the publications cited above is incorporated herein by reference.

Furthermore, the antimicrobial effects of the nitric oxide generating systems of the present invention and the gas evolved therefrom, described above particularly but not exclusively in the sections headed “Uses in therapy or surgery”, “Topical Antimicrobial Uses”, “Nose, Mouth, Respiratory Tract and Lung Uses” and “Targets for Antimicrobial Uses”, are equally applicable to the targeting of microbial infections of plants, and the present invention extends also to such uses.

The above known uses, and all other uses, of nitric oxide and nitric oxide generating systems on plants constitute further aspects of the present invention when used together with the nitric oxide generating reaction using the present invention and/or the nitric oxide, optionally other oxides of nitrogen and/or optionally precursors thereof generated thereby.

The plant being treated may in particular be a crop or domestic plant, namely a plant species cultivated by humans.

Crops include, but are not limited to, crops for food, such as grain, vegetables and fruit, crops for pharmaceutically active ingredients, such as quinine, crops for fibres, such as cotton or flax, crops for other materials, such as rubber and wood, and crops for flowers, such as roses and tulips.

Further examples of crops for human food consumption include, but are not limited to, crops to produce a crop of rice, wheat, sugarcane and other sugar crops, maize (corn), soybean oil, potatoes, palm oil, cassava, legume pulses, sunflower seed oil, rape oil, mustard oil, sorghum, millet, groundnuts, beans, sweet potatoes, bananas, soybeans, cottonseed oil, peanuts, groundnut oil, yams, tomatoes, grapes, onions, apples, coffee, mangos, mangosteens, guavas, chillis, peppers, tea, cucumbers, oranges, walnuts, almonds, carrots, turnips, coconuts, tangerines, lemons, limes, strawberries, and hazelnuts.

### BRIEF DESCRIPTION OF THE DRAWINGS

In the drawings:

FIG. 1 shows a cumulative plot of nitric oxide evolved (nmol NO per mg nitrite) over time in the different reaction conditions of Example 1.

FIGS. 2 to 16 show results from the various tests described in Example 2.

FIG. 17 shows a schematic of the apparatus used for the SIFT-MS measurements.

FIGS. 18 to 21 show results from various tests described in Example 3 with respect to antimicrobial activity against M. abscessus of a combination of known antibiotics, carboxylic acid solutions, carboxylic acid-nitrite solutions and carboxylic acid-nitrite-polyol solutions.

FIG. 22 shows the results from the tests described in Example 4 with respect to the minimum inhibition concentration (MIC) against a large number of clinical isolate cultures for solutions containing citric acid, sodium nitrite and mannitol.

FIG. 23 shows the results from the tests described in Example 5 with respect to antimicrobial activity against Pseudomonas aeruginosa for carboxylic acid-nitrite solutions with and without a polyol.

FIGS. 24 to 27 show the results from the tests described in Example 6 with respect to antimicrobial activity against M. tuberculosis HN 878 in THP-1 cells.

FIG. 28 shows the results from the tests described in Example 7 with respect to cytotoxicity (LDH cytotoxicity assay) and antimicrobial activity against H1N1 Influenza A virus in MDCK cells (a) at MOI=0.002 (●) and MOI=0.02 (▪) at a range of dilutions (the horizontal axis is the nitrite molarity) with the cytotoxicity shown in grey, cytotoxicity scale on the right-hand side (cytoxicity at the measured nitrite concentrations up to and including 0.015 M was ≤1% of LDH control); and (b) plate photographs at MOI=0.002 and nitrite concentrations 0.15 M, 0.015 M and 0.0015 M in comparison with oseltamivir (1 μM). The order of the plates recited in the previous sentence is the same as the order of the plates in the Figure going from left to right (there were two experiments, and the plates of each corresponding experiment are shown one above the other). The far right hand pair of plates, immediately to the right of the oseltamivir pair of plates, is the virus control. The cytotoxicity is shown below each pair of test plates, as the % of LDH control (mean of 3 LDH assays at 24 hours post-infection).

FIG. 29 shows the results of a test of the effectiveness of an acidified solution of sodium nitrite, citric acid buffered to pH 5.8 using sodium hydroxide, and mannitol to kill M. abscessus in comparison with amikacin and negative controls under analogous conditions (described in Example 3).

FIGS. 30 and 31 show in schematic form (FIG. 30) the embodiment of the present invention described in Example 10 for use in treatment of lung infections in a human subject, and (FIG. 31) a view of the point of contact between a liquid NO generating formulation and the lung tissue according to the present invention (right hand side of FIG. 31) in comparison with inhaled gaseous nitric oxide (left hand side of FIG. 31).

FIG. 32 shows the results of the LDH cytotoxicity assay of Example 8 (Runs 1 & 2). The data is expressed as mean+standard deviation (SD) of two experiments. SD shown as the grey error bars. The maximum LDH activity (cells+lysis buffer) was set at 100% and all sample results are relative to this value. The LDH positive control was the positive control from the kit. The black bars (2 hour incubation) are the left-hand bar of each pair of bars in each case, and the red bars (24 hour incubation) are the right-hand bar of each pair of bars in each case.

FIG. 33 shows the results of the antiviral testing against SARS-CoV-2 of Example 8 (Run 1) at MOI 3.0. In Run 1, one virus yield reduction assay was performed using SARS-CoV-2 at four multiplicities of infection (MOIs), confirmed using back titration of the inoculum virus. For cells inoculated with an MOI of 3, 2.1 log 10 TCID50/ml was found in the virus control well after titration. Reduction of SARS-CoV-2 yield might be observed for some of the conditions tested. After 24 hours of incubation, hardly any virus was detected in the lowest three MOIs (i.e. 0.3, 0.03 and 0.003). Possibly, 24 hours of replication on Vero E6 cells is not sufficient for obtaining high levels of progeny virus. The data is expressed as mean+standard deviation (SD) of two titrations. SD shown as the error bars. The horizontal dotted line level with the chloroquine and cell control log 10 TCID50/ml values is the limit of detection (LOD) of the assay.

FIG. 34 shows the results of the antiviral testing against SARS-CoV-2 of Example 8 (Run 2) (a) at MOI 3.0 and (b) at MOI 0.3. The methodology corresponds to the parts of Run 1 at those MOIs, with the exception that the formulations are the Run 2 formulations and incubation was performed for 48 hours rather than 24 hours, in order to increase the level of progeny virus. The data is expressed as mean+standard deviation (SD) of two titrations. SD shown as the error bars. The horizontal dotted line level with the chloroquine and cell control log 10 TCID50/ml values is the limit of detection (LOD) of the assay.

FIG. 35 shows the results of the antiviral testing against SARS-CoV of Example 9 at MOI 3.0. Prior to cell monolayer staining with crystal violet, 2 plates were microscopically checked and scored for cytopathic effect (CPE). A CPE, in the form of cell debris on top of an underlying monolayer, was found to be present in these plates. The results of the two plates, that were microscopically checked, is shown. Data are a single titration per condition. For the remaining plates, no CPE could be scored after crystal violet staining, due to a too dense cell monolayer. The horizontal dotted line level with the cell control log 10 TCID50/ml value is the limit of detection (LOD) of the assay.

### EXAMPLES

The following non-limiting Examples are provided for further illustration of the present invention.

### Materials, Apparatus and Methods Used in Examples 1 and 2

Solutions

Stock solutions of 0.1 and 1 M citric acid (Health Supplies Limited, Thornton Heath, UK), 0.1 M sodium citrate (Fisher Scientific, Loughborough, UK), 1 M sodium nitrite (Sigma Aldrich, Dorset, UK), 0.5 and 1 M sorbitol (Special Ingredients, Chesterfield, UK), 0.5 and 1 M D-mannitol (Sigma Aldrich, Dorset, UK), 3 M sodium hydroxide (Fisher Scientific, Loughborough, UK), and 0.1 and 1 M L-ascorbic acid (ICN Biomedicals Inc., Ohio, US) were prepared by dissolving the appropriate mass in deionised water. Deionised water (18.2 MΩ) was obtained from an Arium Mini lab water system (Sartorius, Germany).

Citric acid/citrate buffer solutions were prepared by two methods:

1. Titrating stock solutions of 0.1 M citric acid and 0.1 M sodium citrate using the volumes described by Sigma Aldrich, 2018 (https://www.sigmaaldrich.com/life-science/core-bioreagents/biological-buffers/learning-center/buffer-reference-center.html);

2. Dissolving a known mass of citric acid, for either a 0.1 M or 1 M preparation, in a small volume of deionised water then titrating a stock solution of 3 M sodium hydroxide and deionised water to achieve the desired buffer solution pH (pH 3 to pH 6.2).

Ascorbic acid/ascorbate buffer solutions were prepared analogously, using ascorbic acid and, for Method 1, sodium ascorbate in place of citric acid and, for Method 1, sodium citrate.

The inclusion of polyols was achieved by dissolving a known mass of sodium nitrite with stock solutions of the polyol (for example, either sorbitol or mannitol).

The order of addition of the ingredients of the buffer solutions and stock is not critical, and any order of mixing can be used.

All standard solutions were used within 48 hours of preparation. Calibration buffer solutions were prepared using phthalate (pH 4) and phosphate (pH 7) tablets (Fisher Scientific UK Ltd, Leicestershire, UK) dissolved in deionised water.

Selected Ion Flow Tube Mass Spectrometry (SIFT-MS) Start-Up and Validation

A Voice200 Selected Ion Flow Tube Mass Spectrometer (SIFT-MS) (Syft Technologies Ltd, New Zealand) was used for all the gas analyses described in this report. This instrument uses helium (BOC, Surrey, UK) as the carrier gas.

Prior to analysis, the SIFT-MS was prepared for use with a simple start up procedure. The instrument was taken out of standby mode and a series of pressure checks were made to ensure that capillary flow is within the acceptable range for operation. This was followed by an automated validation procedure using the manufacturer's calibrant gas standard (Syft Technologies Ltd, New Zealand) containing benzene, toluene, ethylbenzene, and xylene. Finally, an in-house performance check was undertaken using a 10 ppm nitrogen dioxide standard (Air Products PLC, Surrey, UK).

Procedure for the Generation of the NO

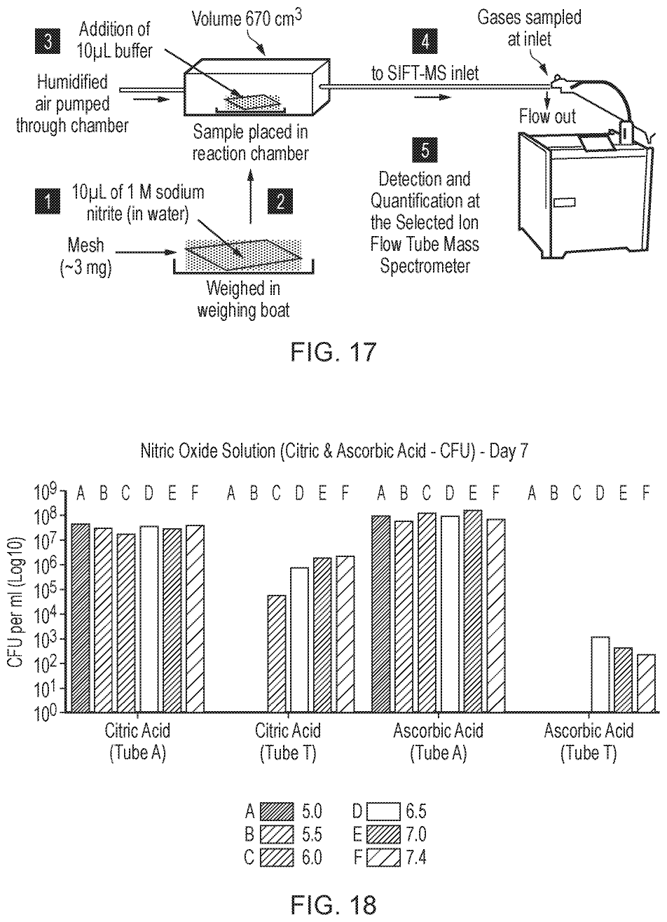

The SIFT-MS equipment, reaction chamber and gas pathway was set up as illustrated in FIG. 17.

The temperature in the reaction chamber was continuously monitored with a HT1 Temperature Smart Sensor (SensorPush, New York, US). The reaction chamber, a 670 mL plastic (bisphenol A free (BPA free)) clip lock tub with silicone seal (Tesco, Welwyn Garden City, UK) was attached to a pump that continuously cycles humid air through the chamber and over the SIFT-MS inlet capillary. Humidification was achieved by pumping air through two Dreschel bottles containing deionised water in a method analogous to that described by Vernon, W., and Whitby, L. (1931) The quantitative humidification of air in laboratory experiments, Trans. Faraday Soc. 27, 248-255. This system was allowed to equalise for 30 minutes before use. A continuous SIFT-MS scan was begun for the real-time detection and quantification of NO, NO₂, and HONO. Once a stable baseline reading was observed (consistent concentration for >2 minutes) for these compounds, the sample was placed in the reaction chamber and monitored for three hours.

After SIFT-MS validation the capillary inlet extension heated to 120° C. was attached to the outlet of the reaction chamber via a T-junction, allowing the SIFT-MS to sample the gases flowing out from the reaction chamber in real time.

The sample was prepared by weighing a circa 0.3 cm×0.3 cm carded non-woven 20 grams per square metre (20 gsm) polypropylene mesh from RKW-Group, Frankenthal, Germany in a weighing boat (˜3 mg). This was reweighed after an addition of a 10 μL droplet of test or control solution onto the centre of the mesh (it was ensured that the droplet soaked into the mesh). Finally, the loaded mesh in the weighing boat was placed in the reaction chamber and a final 10 μL droplet of buffer solution was pipetted onto the centre of the mesh. The reaction chamber was promptly sealed and the generation of nitrogenous species was observable instantaneously at the SIFT-MS interface.

Analysis of Generated Gas

The generated gas was analysed using the selected ion mode of the SIFT-MS and scans were performed in sequential batches each lasting 1000 seconds. The following product masses were repeatedly scanned for: 30 m/z for nitrous acid, 48 m/z for nitrous acid, 46 m/z for nitrogen dioxide, and 30 m/z for nitric oxide. These measurements were achieved using all three of the positive precursor ions: hydronium (H₃O⁺, nitrosium (NO⁺, and dioxygenyl (O₂+). The air flowed through the chamber at 660 ml/min and the SIFT-MS inlet sampled this air stream at a flow rate of 2.7 ml/min.

### pH Measurements for All Examples

A Five Easy pH meter (Mettler Toledo, Switzerland) with a glass electrode, LE438 probe, was used for all pH measurements. The accuracy of this electrode was ensured with a second pH meter; the hand-held 205 probe (Testo, Alton, US). Fresh calibrant buffer solutions were used for daily calibration of the pH meters.

### Example 1

Generation of Nitric Oxide Using 1 M/c. pH 3 Citric Acid Contacting a Mesh Containing Imbibed 1 M Sodium Nitrite with and without 1 M Polyols

The SIFT-MS equipment, reaction chamber and gas pathway was set up as described above and illustrated in FIG. 17.

Two test solutions of 1 M sodium nitrite containing respectively 1 M mannitol and 1 M sorbitol were imbibed into the mesh as described above to make two test meshes.

A control solution of 1 M sodium nitrite with no polyol was imbibed into the mesh as described above to make a control mesh.

A buffer solution of 1 M citric acid/citrate buffer prepared by either of the two methods 1 and 2 described above and having a pH of about 3 was added to each of the test and control meshes in each test to initiate gas generation as described above.

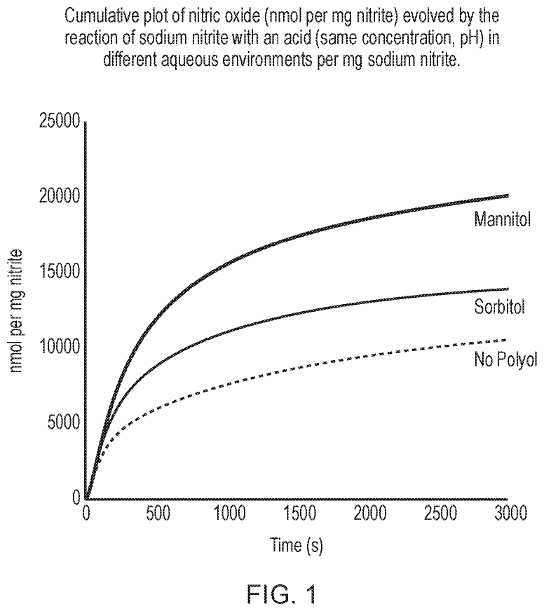

The results are shown in FIG. 1.

The data show that the 1 M sodium nitrite imbibed mesh contacted with 1 M/c. pH 3 citric acid generated markedly greater amounts of nitric oxide when the mesh also contained 1 M mannitol or 1 M sorbitol (mannitol has a greater effect than sorbitol) than when no polyol was present.

### Example 2

Investigation of the Effects of Different Carboxylic Acids, Acid Concentration, pH and Polyols on the Generation of Nitric Oxide

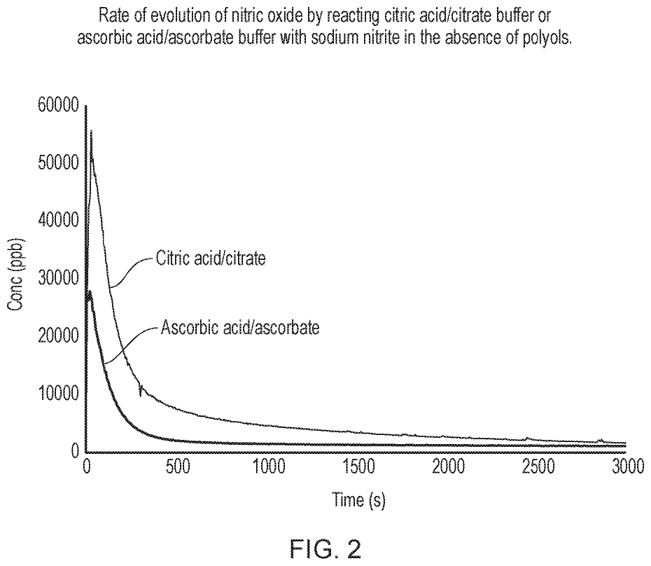

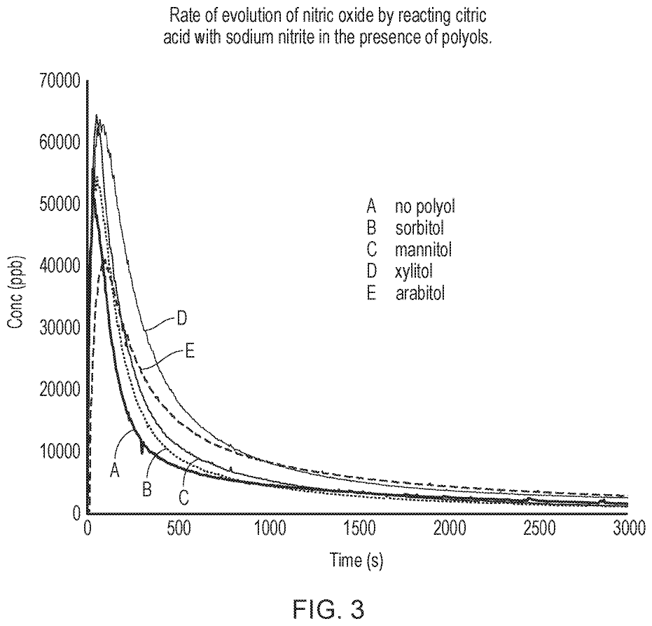

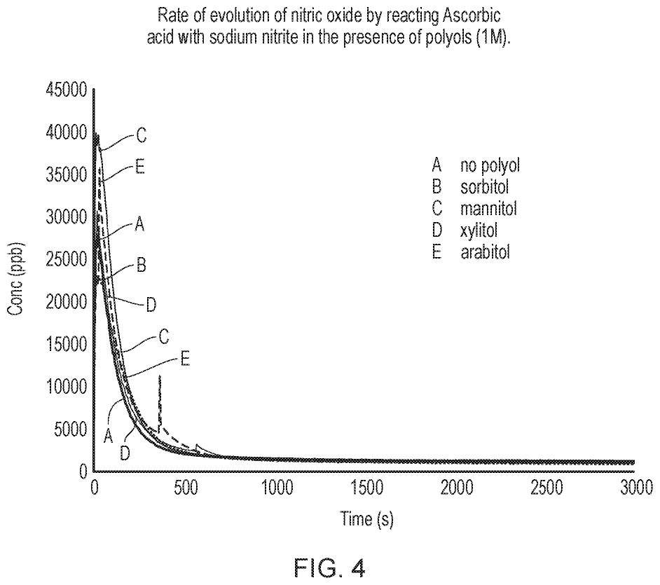

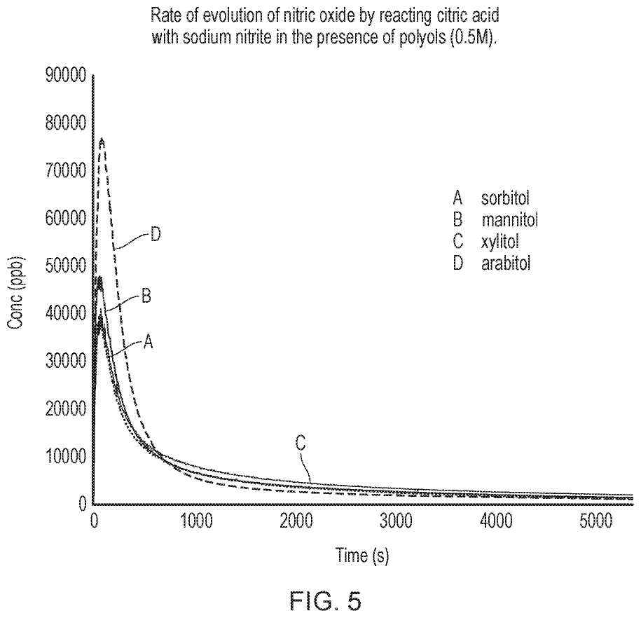

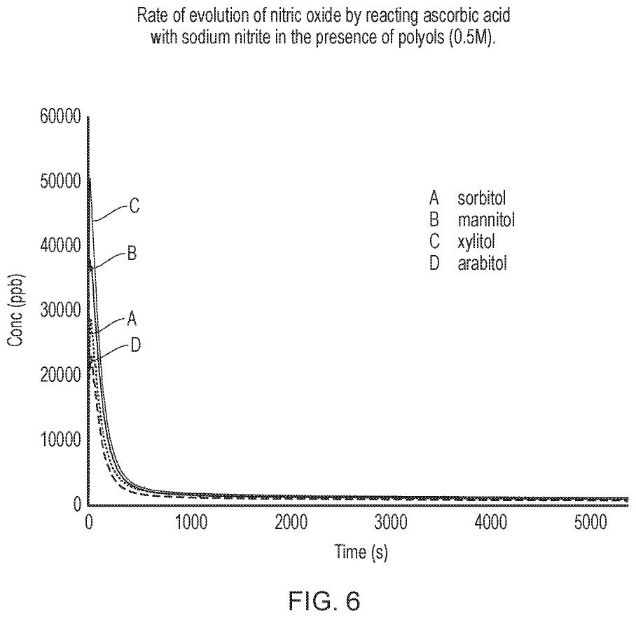

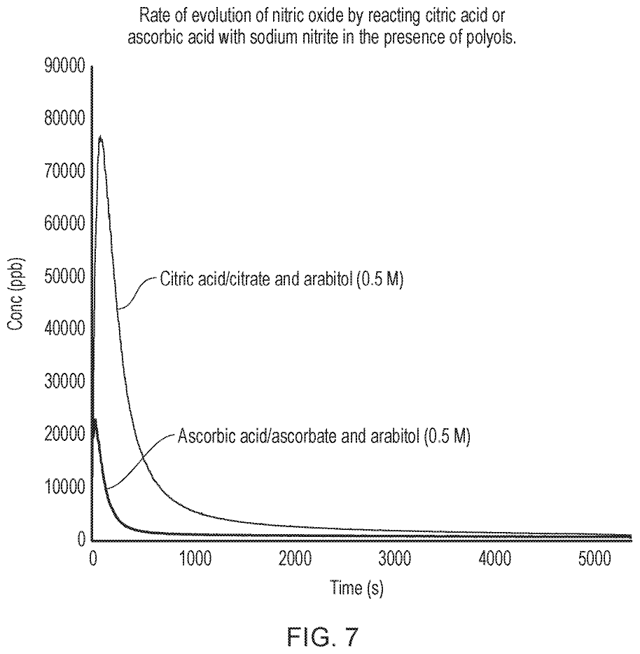

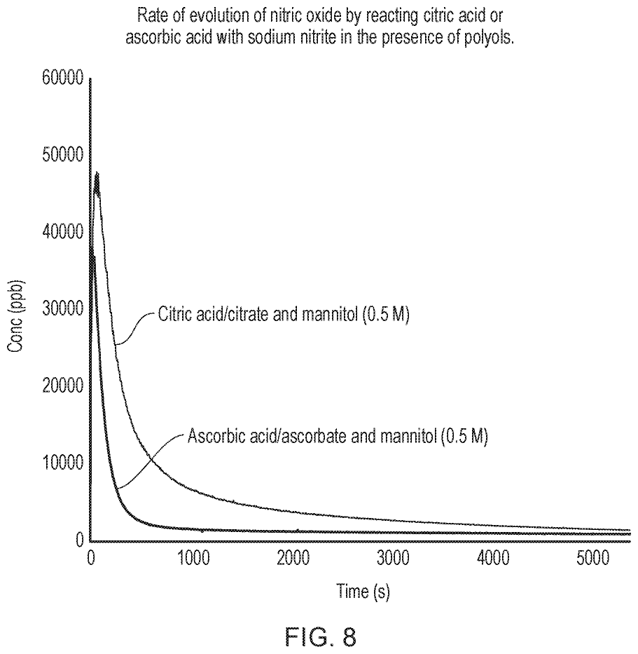

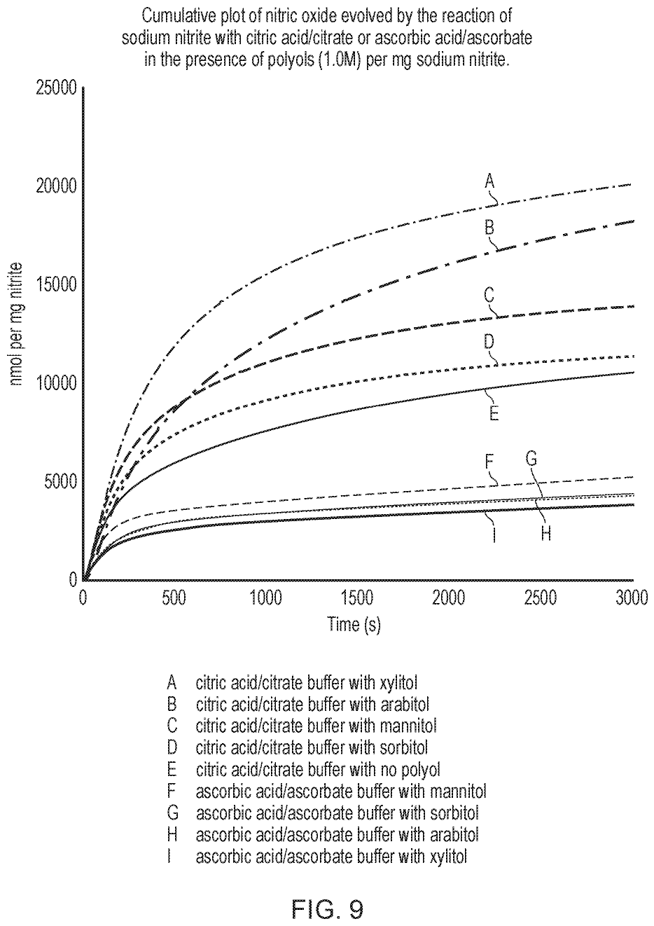

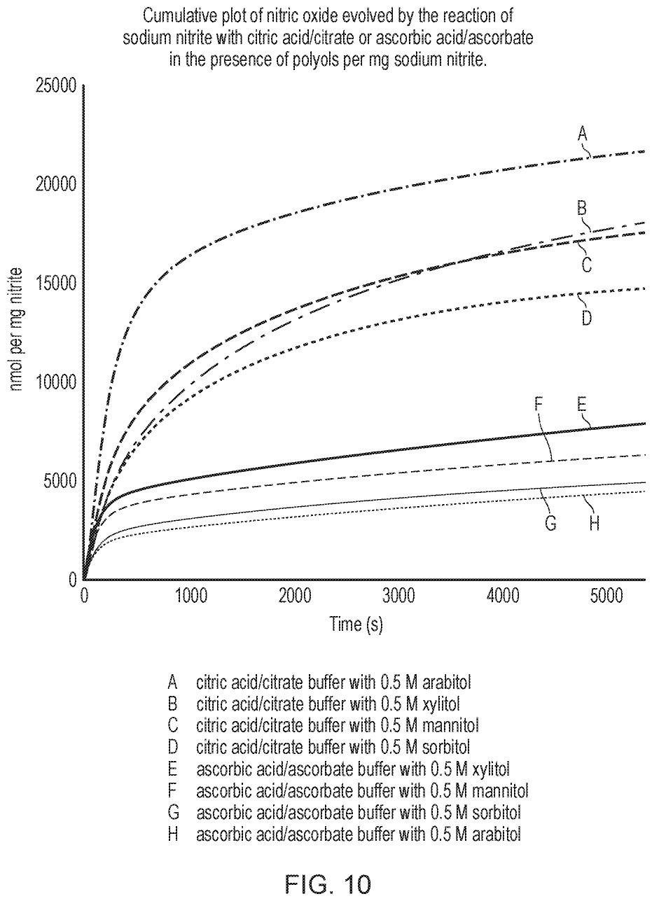

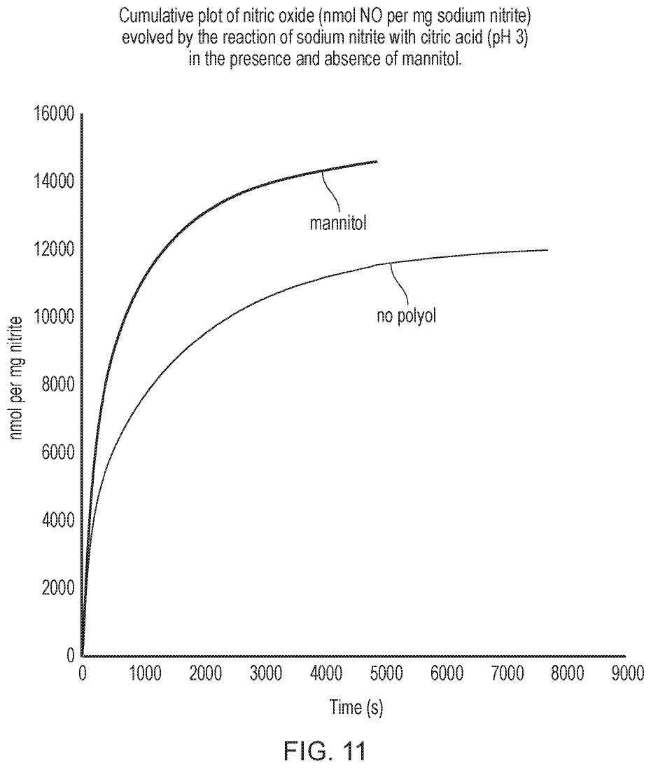

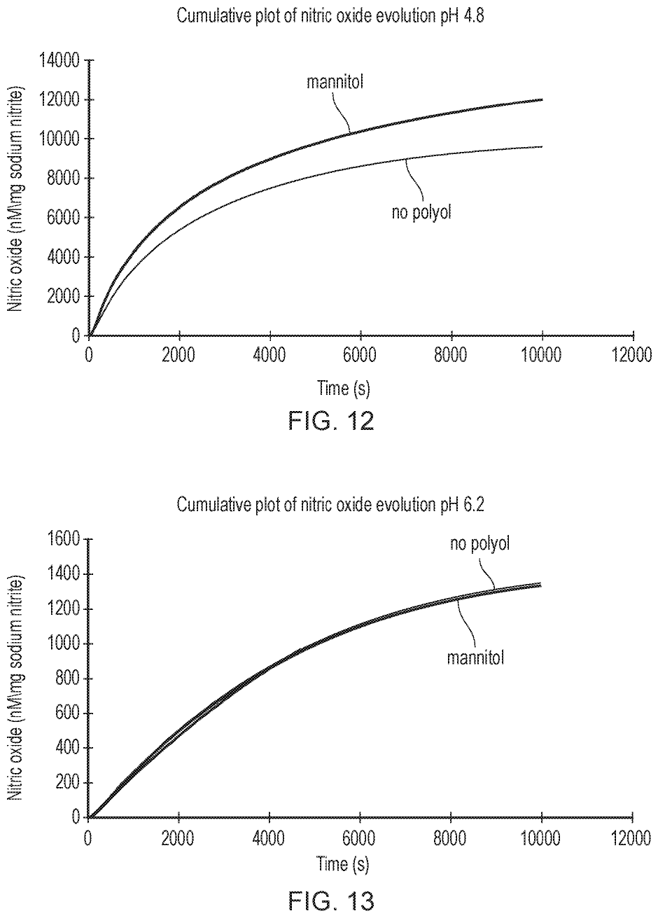

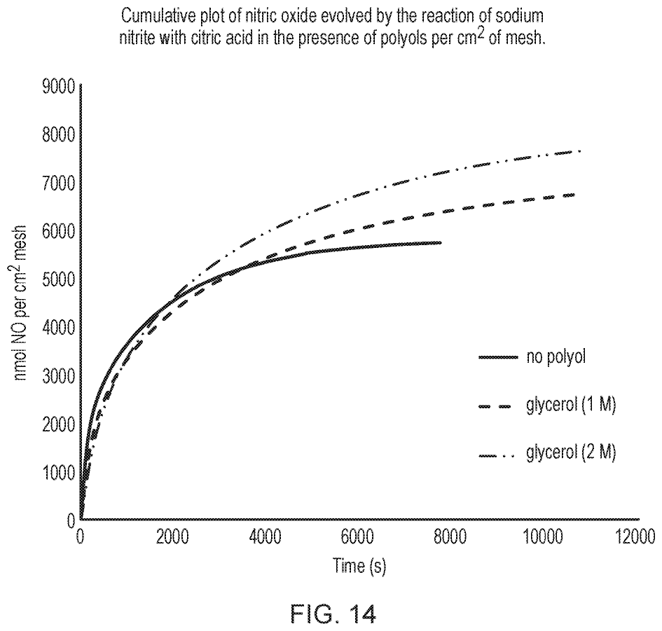

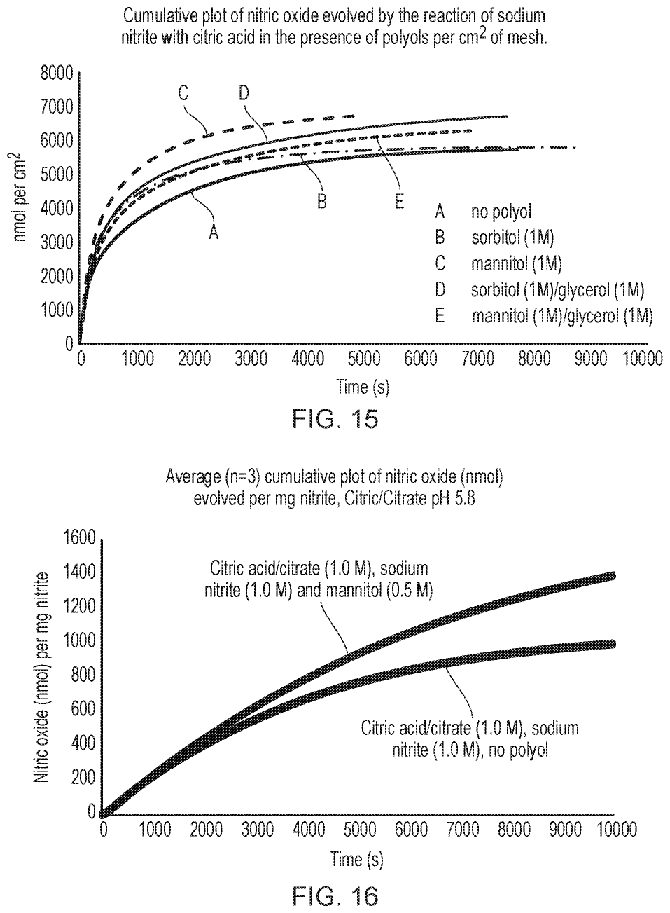

Samples were prepared as above, varying the organic acid, pH and polyol as follows:

| | | | Buffer added to mesh |
|---|---|---|---|
| | | | (where alternative buffers |
| | | | are indicated they are used |
| | Test solution | Control solution | in separate runs, as |
| | imbibed into mesh | imbibed into mesh | reported in the relevant |
| Experiment | in each test run | in control run | Figure) |
| A (FIG. 2) | 1 M sodium nitrite | — | 1 M citric acid/citrate |
| | | | (pH about 3) |
| | | | 1 M ascorbic acid/ascorbate |
| | | | (pH about 3) |
| B (FIG. 3) | 1 M sodium nitrite | 1 M sodium nitrite | 1 M citric acid/citrate (pH about 3) |
| | containing 1 M sorbitol | | |
| | 1 M sodium nitrite | | |
| | containing 1 M | | |
| | mannitol | | |
| | 1 M sodium nitrite | | |
| | containing 1 M | | |
| | xylitol | | |
| | 1 M sodium nitrite | | |
| | containing 1 M | | |
| | arabitol | | |
| C (FIG. 4) | 1 M sodium nitrite | 1 M sodium nitrite | 1 M ascorbic acid/ascorbate |
| | containing 1 M | | (pH about 3) |
| | sorbitol | | |
| | 1 M sodium nitrite | | |
| | containing 1 M | | |
| | mannitol | | |
| | 1 M sodium nitrite | | |
| | containing 1 M | | |
| | xylitol | | |
| | 1 M sodium nitrite | | |
| | containing 1 M | | |
| | arabitol | | |
| D (FIG. 5) | 1 M sodium nitrite | — | 1 M citric acid/citrate |
| | containing 0.5 M | | (pH about 3) |
| | sorbitol | | |
| | 1 M sodium nitrite | | |
| | containing 0.5 M | | |
| | mannitol | | |
| | 1 M sodium nitrite | | |
| | containing 0.5 M | | |
| | xylitol | | |
| | 1 M sodium nitrite | | |
| | containing 0.5 M | | |
| | arabitol | | |
| E (FIG. 6) | 1 M sodium nitrite | — | 1 M ascorbic acid/ascorbate |
| | containing 0.5 M | | (pH about 3) |
| | sorbitol | | |
| | 1 M sodium nitrite | | |
| | containing 0.5 M | | |
| | mannitol | | |
| | 1 M sodium nitrite | | |
| | containing 0.5 M | | |
| | xylitol | | |
| | 1 M sodium nitrite | | |
| | containing 0.5 M | | |
| | arabitol | | |
| F (FIG. 7) | 1 M sodium nitrite | — | 0.5 M citric acid/citrate |
| | containing 1 M | | (pH about 3) |
| | arabitol | | 0.5 M ascorbic |
| | | | acid/ascorbate (pH about 3) |
| G (FIG. 8) | 1 M sodium nitrite | | 0.5 M citric acid/citrate |
| | containing 1 M | | (pH about 3) |
| | mannitol | | 0.5 M ascorbic |
| | | | acid/ascorbate (pH about 3) |
| H (FIG. 9) | 1 M sodium nitrite | 1 M sodium nitrite | 1 M citric acid/citrate |
| | containing 1 M | | (pH about 3) |
| | sorbitol | | 1 M ascorbic acid/ascorbate |
| | 1 M sodium nitrite | | (pH about 3) |
| | containing 1 M | | |
| | mannitol | | |
| | 1 M sodium nitrite | | |
| | containing 1 M | | |
| | xylitol | | |
| | 1 M sodium nitrite | | |
| | containing 1 M | | |
| | arabitol | | |
| L (FIG. 10) | 1 M sodium nitrite | | 1 M citric acid/citrate |
| | containing 0.5 M | | (pH about 3) |
| | sorbitol | | 1 M ascorbic acid/ascorbate |
| | 1 M sodium nitrite | | (pH about 3) |
| | containing 0.5 M | | |
| | mannitol | | |
| | 1 M sodium nitrite | | |
| | containing 0.5 M | | |
| | xylitol | | |
| | 1 M sodium nitrite | | |
| | containing 0.5 M | | |
| | arabitol | | |
| J (FIG. 11) | 1 M sodium nitrite | 1 M sodium nitrite | 0.5 M citric acid/citrate |
| | containing 0.5 M | | (pH about 3) |
| | mannitol | | |
| K (FIG. 12) | 1 M sodium nitrite | 1 M sodium nitrite | 0.5 M citric acid/citrate |
| | containing 0.5 M | | (pH about 4.8) |
| | mannitol | | |
| L (FIG. 13) | 1 M sodium nitrite | 1 M sodium nitrite | 0.5 M citric acid/citrate |
| | containing 0.5 M | | (pH about 6.2) |
| | mannitol | | |
| M (FIG. 14) | 1 M sodium nitrite | 1 M sodium nitrite | 1 M citric acid/citrate |
| | containing 1 M | | (pH about 2) |
| | glycerol | | |
| | 1 M sodium nitrite | | |
| | containing 2 M | | |
| | glycerol | | |
| N (FIG. 15) | 1 M sodium nitrite | 1 M sodium nitrite | 1 M citric acid/citrate |
| | containing 1 M | | (pH about 2) |
| | mannitol | | |
| | 1 M sodium nitrite | | |
| | containing 1 M | | |
| | sorbitol | | |
| | 1 M sodium nitrite | | |
| | containing 1 M | | |
| | mannitol and 1 M | | |
| | glycerol | | |
| | 1 M sodium nitrite | | |
| | containing 1 M | | |
| | sorbitol and 1 M | | |
| | glycerol | | |
| O (FIG. 16) | 1 M sodium nitrite | 1 M sodium nitrite | 1 M citric acid/citrate |
| | containing 0.5 M | | (pH 5.8) |
| | mannitol | | |

The SIFT-MS equipment, reaction chamber and gas pathway was set up as described above and illustrated in FIG. 17.

Test solutions as described above were imbibed into the mesh as described above to make the test meshes.

Where used, a control solution of 1 M sodium nitrite with no polyol was imbibed into the mesh as described above to make a control mesh.

The or each buffer solution as described above prepared by either of the two methods 1 and 2 described above and having the pH described above was added to each of the test and, if used, control meshes in each test to initiate gas generation as described above.

The results are shown in FIGS. 2 to 13. “Normal” in the Figures refers to no polyol being present.

FIG. 2 compares the rate of NO evolution as produced by citric acid/citrate buffer or ascorbic acid/ascorbate buffer (pH circa 3) in the absence of a polyol. The graphs clearly show that citric acid/citrate buffer generates a higher initial burst and the evolution last at for longer at a higher level than for ascorbic acid/ascorbate buffer. The citric acid/citrate buffer trace peaks at about 55000 ppb whereas the ascorbic acid/ascorbate buffer trace peaks at about 28000 ppb.

FIG. 3 relates to a citric acid/citrate buffer and nitrite system with and without polyols. Polyol concentration is 1 M. The rates of evolution, initial burst and consequent release over time are altered in the presence of polyols when compared to no polyol. Xylitol and mannitol produce the highest peak, followed by sorbitol, then no polyol, and then arabitol. In the 500-1000s region xylitol and arabitol have the highest outputs, followed by mannitol, sorbitol and then no polyol. Peak burst mannitol=xylitol (about 64000 ppb)>sorbitol (about 53000 ppb)>no polyol (about 50000 ppb)>arabitol (about 40000 ppb).

FIG. 4 relates to an ascorbic acid/ascorbate buffer and nitrite system, with and without polyols. Polyol concentration is 1 M. Peak burst mannitol (about 40000 ppb)>arabitol (about 35000 ppb)>xylitol=no polyol (about 30000 ppb)>sorbitol (about 23000 ppb), i.e. a different sequence to the citric acid/citrate buffer system of FIG. 3.

FIG. 5 relates to a citric acid/citrate buffer and nitrite system, with and without polyols (the “no polyol” line, which has a peak burst approximately the same as the mannitol line, has been omitted for clarity). Polyol concentration is 0.5 M. Peak burst arabitol (about 76000 ppb)>>no polyol=mannitol (about 48000 ppb)>xylitol=sorbitol (about 40000 ppb). It will be seen that this is a different sequence compared to the analogous 1 M polyol citric acid/citrate buffer system (FIG. 3), showing that the polyol effect is polyol-concentration dependent.

FIG. 6 relates to an ascorbic acid/ascorbate buffer and nitrite system, with and without polyols (the “no polyol” line, which has a peak burst approximately the same as the sorbitol line, has been omitted for clarity. Polyol concentration is 0.5 M. Peak burst xylitol (about 50000 ppb)>mannitol (about 38000 ppb)>sorbitol=no polyol (about 30000 ppb)>arabitol (about 23000 ppb). Again, a different sequence is observed in comparison with the analogous citric acid/citrate buffer (0.5 M polyol) and ascorbic acid/ascorbate (1 M polyol) systems (FIGS. 5 and 4 respectively). The polyol effect is thus demonstrated to be polyol-chemistry/stereo-chemistry and polyol-molarity dependent.

FIGS. 7 and 8 compare the rate of NO evolution with citric acid/citrate buffer or ascorbic acid/ascorbate buffer and the presence of a polyol (0.5 M). These graphs emphasise some of the differences observed in the FIGS. 2 to 6. The citric acid/citrate buffer trace in FIG. 7 peaks at about 76000 ppb whereas the ascorbic acid/ascorbate buffer trace peaks at about 22000 ppb. The citric acid/citrate buffer trace in FIG. 8 peaks at about 48000 ppb whereas the ascorbic acid/ascorbate buffer trace peaks at about 38000 ppb.

FIG. 9 compares cumulative outputs for 1 M polyol concentrations. The differences at say 3000s for ascorbic acid/ascorbate buffer are small, in order mannitol>sorbitol=arabitol>xylitol. For citric acid/citrate buffer at 3000s the order is xylitol>arabitol>mannitol>sorbitol>no polyol. The data show that the output of nitric oxide can be increased by up to, or even more than, about 100%, for example as between no polyol (curve E, which obtains a cumulative nitric oxide evolution of about 10000 nmol per mg nitrite after 3000 s, which is even then still rising) and xylitol (curve A, which obtains a cumulative nitric oxide evolution of about 20000 nmol per mg nitrite after the same time, which also is still rising).

FIG. 10 compares cumulative outputs for 0.5 M polyol concentrations. For citric acid/citrate buffer at 3000s the order is arabitol>mannitol=xylitol>sorbitol>no polyol (the “no polyol” line for citric acid/citrate buffer, lying below the sorbitol line, has been omitted for clarity). For ascorbic acid/ascorbate buffer at 3000s the order is xylitol>mannitol>sorbitol>arabitol. Again this order is different compared to 1 M polyol (FIG. 9).

FIGS. 11 to 13 compare the cumulative plots for citric acid/citrate buffer 1M and sodium nitrite (1M), with and with mannitol (0.5M) and at different pH. As the pH increases the differences become smaller and at pH 6.2 the differences have disappeared. So it is seen from these experiments that the polyol effect is also pH dependent.

FIG. 14 shows the cumulative NO (nmol/cm² mesh area) output for citric acid/citrate buffer (1M, pH circa 2) with and without glycerol (1M and 2M) present in the 1M sodium nitrite solution. Over the first 2000s the NO outputs for 1M and 2M glycerol are slightly lower than for no polyol present. At longer times the glycerol containing formulations have greater output with the 2M glycerol having the greater output.

FIG. 15 shows the cumulative NO (nmol/cm² mesh area) output for citric acid/citrate buffer (1M, pH circa 2) and 1M sodium nitrite solutions, with or without polyols present in the nitrite solution. The plots show that the inclusion of glycerol in mannitol/nitrite solutions reduces the output compared to when no glycerol is present. Surprisingly, however, unlike the case for mannitol, the inclusion of glycerol in sorbitol/nitrite solutions enhances the NO output compared to the output when no glycerol is present.

When glycerol was used a 1M glycerol solution was first made and used to make 1M sorbitol or 1M mannitol solution which in turn was used to make 1M nitrite solution.

FIG. 16 shows the cumulative NO output (mol/mg nitrite) for citric acid/citrate buffer (1M, pH 5.8), with and without mannitol (0.5M) present in the sodium nitrite (1M) solution. The plots show that the inclusion of the polyol gives rise to a greater NO output after circa 2000s reaction time.

FIG. 16 shows that, at physiologically important pH levels of greater than about 5, particularly greater than about 5.5, mannitol enhances the generation of nitric oxide in comparison with the same system without mannitol, providing cumulative levels of 1400 nmol NO per mg nitrite after 10000 s (167 minutes).

### Example 3

Activity against M. abscessus Cultures with Various Organic Acid and Nitrite Solutions With and Without Polyols

Materials

4.7 g Middlebrook 7H9 broth base (Sigma-Aldrich) was reconstituted with 900 ml of distilled water and autoclaved at 121° C. for 15 minutes. Middlebrook ADC growth supplement (Sigma-Aldrich) was added to the autoclaved 7H9 solution (50 ml per 450 ml, total of 100 ml added).

1M Sodium nitrite (Emsure): Dissolve 6.9 g of sodium nitrite powder in 100 ml of distilled water in a clean screw top glass bottle. Autoclave the mixture at 121° C. for 15 minutes.

1M Citric acid (Sigma-Aldrich): Dissolve 19.2 g of Citric acid powder in 100 ml of distilled water in a clean screw top glass bottle. Autoclave the mixture at 121° C. for 15 minutes.

1M Ascorbic acid (Sigma-Aldrich): Add 17.6 g of Ascorbic acid powder to a sterile glass bottle. Dissolve thoroughly in 100 ml of sterilised distilled water. Due to its short half-life it was prepared on a daily basis, using strict sterile techniques. It was not autoclaved due to its inherent instability but was filtered through a 0.2μ filter before use.

1M Sodium citrate tribasic dihydrate (Sigma-Aldrich): Dissolve 29.4 g of sodium citrate powder in 100 ml of distilled water in a clean screw top glass bottle. Autoclave the mixture at 121° C. for 15 minutes. 1M L-Ascorbic acid sodium salt (Acros Organics): Dissolve 19.8 g of sodium ascorbate powder in 100 ml of distilled water in a clean screw top glass bottle. Autoclave the mixture at 121° C. for 15 minutes.

For the experiments with polyols, D-mannitol (Sigma-Aldrich) was used. The polyol was added to the sodium nitrite stock solution described above to form the following stock solutions:

Stock solution A— 1M sodium nitrite & 0.5M mannitol

Stock solution B— 1.5M sodium nitrite & 0.5M mannitol

A stock solution of 1.5M citric acid was also prepared.

The molarity of each component was adjusted for dilution factors to ensure the correct final molarity of each experimental solution.

Mycobacterium abscessus (MAB)

Laboratory reference strain Mycobacterium abscessus ATCC 19977 lux was used for all experimental conditions in this example.

Methodology 50 ml falcon tubes were labelled Tube T (test suspension), Tube A (acid control) and Tube C (control).

8 ml of 7H9+ADC supplement was added to each tube. 100 μl of MAB suspension (grown previously to approximately 3-4 McFarland standard) was then added. The baseline relative light unit (RLU) reading of the MAB suspension was taken. The contents were mixed by vortexing.

Tube Contents when a Polyol (Mannitol) was Not Present

Tube T: 1 ml of sodium nitrite (1M) solution were added to the tube, immediately followed by 1 ml citric acid solution (1M) or ascorbic acid solution (1M) to give a final concentration of 0.1M in 10 ml. The contents were mixed by gentle inversion and incubated for 24 hours at 37° C.

Tube A: 1 ml of citric acid solution (1M) or ascorbic acid solution (1M) were added to the tube, and 1 ml of sterile distilled water to produce a final volume of 10 ml to test a 0.1M concentration to acid. The contents were mixed by gentle inversion and incubated for 24 hours at 37° C.

Tube C: 2 ml of sterile distilled water were added to the tube to make a total volume of 10 ml. This is the control to assess growth under optimal conditions. The contents were mixed by gentle inversion and incubated for 24 hours at 37° C.

Tube T Contents when a Polyol (Mannitol) was Present

When mannitol was present the tube T contents were as follows:
1. Tube T: 1 ml sodium nitrite (1M) & mannitol (0.5M) and 1 ml of citric acid (1M)
2. Tube T: 1 ml sodium nitrite (1.5M) & mannitol (0.5M) and 1 ml of citric acid (1M)
3. Tube T: 1 ml sodium nitrite (1M) & mannitol (0.5M) and 1 ml of citric acid (1.5M)

RLUs were measured at 30 minutes, 60 minutes and 24 hours incubation to assess the activity of the T, A and C solutions.

Following 24 hours of incubation Tube C, Tube A and Tube T were plated on to Columbia Blood Agar (VWR Chemicals). The plates were incubated at 37° C. for 72 hours. Colony forming units (CFU) were read at day 3, 5 and 7 of incubation. All work was undertaken in a CL2 biological safety cabinet within a CL2 laboratory facility.

The results are shown in FIGS. 18 to 21.

FIG. 18 shows that a solution of 0.1M citric acid and 0.1M nitrite (Tube T) is effective at eliminating the M. abscessus culture after 7 days pH of 5 and 5.5 and reducing the M. abscessus cultures compared to the 0.1M citric acid only solution (Tube A) at pH values of 6.0, 6.5, 7.0 and 7.4. FIG. 18 also shows that a solution of 0.1M ascorbic acid and 0.1M nitrite (Tube T) is effective at eliminating the M. abscessus culture after 7 days at pH values of 5.0, 5.5, and 6.0, and reducing the M. abscessus cultures compared to the ascorbic acid only solution (Tube A) at pH values of 6.5, 7.0 and 7.4.

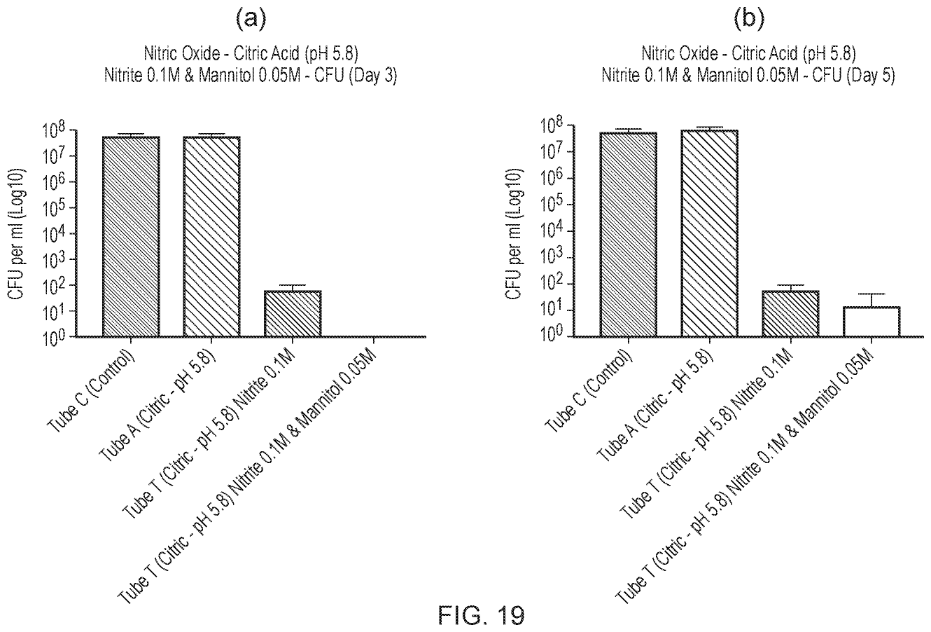

FIG. 19 a) shows that a solution of 0.1 M citric acid and 0.1 M nitrite is effective at reducing the CFU of the M. abscessus culture after three days of incubation and a solution of 0.1 M citric acid and 0.1 M nitrite with 0.05 M mannitol is effective at almost entirely eliminating the M. abscessus culture after three days of incubation. FIG. 19 b) shows that a solution of 0.1 M citric acid and 0.1 M nitrite without mannitol is effective at maintaining a reduced CFU of M. abscessus after five days of incubation. The Figure also shows that the solution of 0.1 M citric acid and 0.1 M nitrite with 0.05 M mannitol is effective at reducing the CFU of M. abscessus culture after five days of incubation.

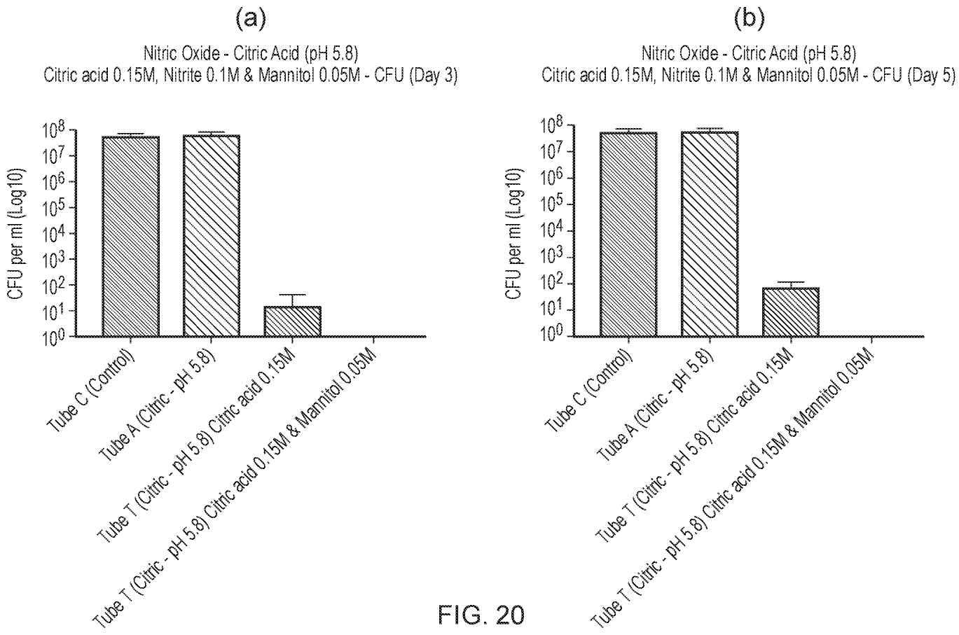

FIG. 20 a) shows that a solution of 0.15 M citric acid and 0.1 M nitrite is effective at reducing the CFU of the M. abscessus culture after three days of incubation and a solution of 0.15 M citric acid and 0.1 M nitrite with 0.05 M mannitol is effective at eliminating the M. abscessus culture after three days of incubation. FIG. 20 b) shows that the solution of 0.15 M citric acid and 0.1 M nitrite without mannitol is effective at maintaining a reduced CFU of M. abscessus after five days of incubation. The figure also shows that the solution of 0.15 M citric acid and 0.1 M nitrite with 0.05 M mannitol is effective at eliminating the M. abscessus culture after five days of incubation.

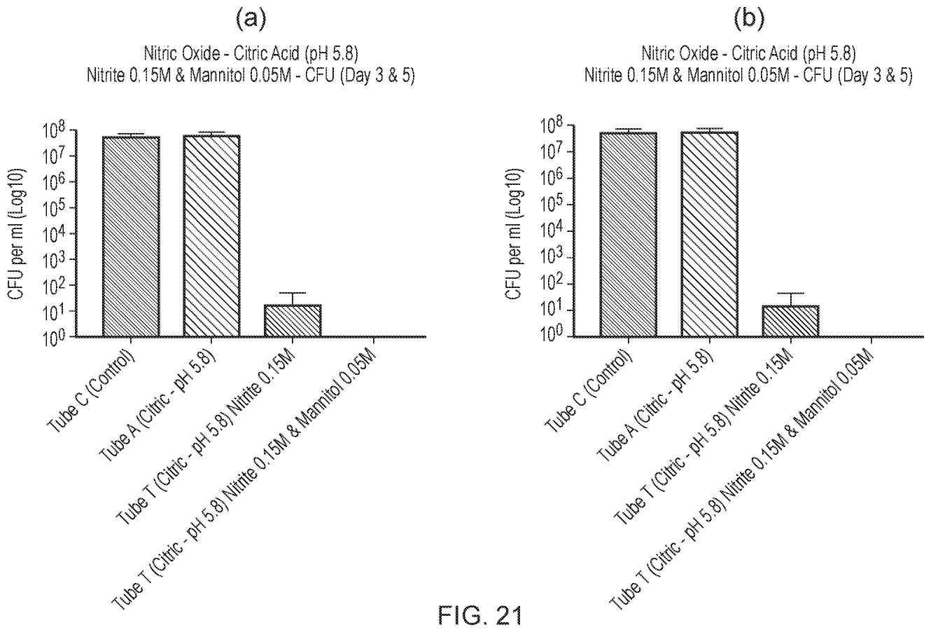

FIG. 21 shows that a solution of 0.1 M citric acid and 0.15 M nitrite is effective at reducing the CFU of the M. abscessus culture after three days of incubation and maintaining the reduction of CFU of the M. abscessus culture after 5 days of incubation. The figure also shows that a solution of 0.1 M citric acid and 0.15 M nitrite with 0.05 M mannitol is effective at eliminating the M. abscessus culture after three and five days of incubation.

### Example 4

Minimum inhibition concentrations (MIC) of carboxylic acid-nitrite-polyol solutions against Mycobacterium abscessus (Mabs) and Mycobacterium tuberculosis (Mtb) in a range of clinical isolate cultures

Healthy Volunteers

Peripheral blood samples were taken from healthy volunteers who had provided written informed consent (ethical approval reference REC No. 12/WA/0148).

Mycobacterial Strains

Mycobacterium abscessus (ATCC 19977) and Mycobacterium tuberculosis (H37RV) strains both contained a bacterial luciferase (lux) gene cassette (luxCDABE) which enabled measurement of relative light units (RLU), as well as conventional colony forming unit (CFU) measurement of bacterial survival.

General Reagents

| | Reference | Supplier |
|---|---|---|
| 24 Well Cell Culture Cluster | 3526 | Costar Coming, USA |
| CD14 microbeads, human | 130-150-201 | Miltenyi Biotec, UK |
| Citric acid | 791725 | Sigma, UK |
| Columbia Blood Agar plates | 100253ZF | vWR, UK |
| Decanal | D7384 | Sigma, UK |
| Dulbecco's Modified Eagle | D6429 | Sigma, UK |
| Medium-High Glucose | | |
| FLUOstar Omega | | BMG Labtech, UK |
| Foetal Bovine Serum | P30-3702 | Pan-Biotech, UK |
| GloMax-96 Luminometer | | Promega, UK |
| Mannitol | M4125 | Sigma, UK |
| Middlebrook 7H11 agar plates | PP4080 | E & O Labs, UK |
| Middlebrook 7H9 broth | M02178 | Sigma, UK |
| Mycobacterium abscessus | 19977 | ATCC |
| Mycobacterium tuberculosis | H37RV | ATCC |
| Penicillin Streptomycin | P0781 | Sigma, UK |
| Recombinant Human GM-CSF | 300-03 | PeproTech EC, UK |
| Recombinant Human IFNγ | 300-02 | PeproTech EC, UK |
| Sodium Nitrite | 1.06549.0500 | Merck, Germany |

Treatment Conditions

Treatment 1: Citric acid 0.15M, sodium nitrite 0.1M and mannitol 0.05M

Treatment 2: Citric acid 0.1M, sodium nitrite 0.15M and mannitol 0.05M

Broth Microdilution Minimum Inhibitory Concentration (MIC)

The MIC for each treatment against M. abscessus and M. tuberculosis was undertaken according to the guidelines (M07-A9) produced by the Clinical and Laboratory Standards Institute for antimicrobial susceptibility testing. Doubling dilutions of each treatment was made across the plates, and the plates incubated at 37° C., and read at day 3 and 7 for Mabs, and days 14 and 21 for Mtb. Testing was undertaken in duplicate.

All work was undertaken in a CL2 biological safety cabinet within a CL2 laboratory facility.

It was found that the minimum inhibitory concentration for a 1.5 M citric acid, 1 M sodium nitrite and 0.5 M mannitol solution against M. abscessus is 4.7 mM. It was further found that the minimum inhibitory concentration for a 1.5 M citric acid, 1 M sodium nitrite and 0.5 M mannitol solution against M. tuberculosis is 2.3 mM.

It was found that the minimum inhibitory concentration for a 1 M citric acid, 1.5 M sodium nitrite and 0.5 M mannitol solution against M. abscessus is 3.1 mM. It was further found that the minimum inhibitory concentration for a 1 M citric acid, 1.5 M sodium nitrite and 0.5 M mannitol solution against M. tuberculosis is 1.6 mM.

Minimal inhibitory concentration (MIC) was also carried out by broth microdilution using isolates Nos. 570, 571, 573, 575, 578, 579, 580, 581, 582, 583, 584, 585, 589, 591, 592, 593, 594, 595, 596, 597, 598, 599, 600, 601, 602, 603, 604, 605, 606, 607, 608, 616, 617, 619, 812, 825, 829, 839, 845, 848, 853, 857, 858, 873, 894, 898, 909, 919, 928, 932, 942, 944, 955, 956, 959, 963, 964, 965, 968, 975, 980, 982, 985, 993, 995, 1000, 1001, 1007, 1011, 1017, 1023, 1024, 1026, 1027, 1042, 1043, 1045, 1047, 1049, 1054, 1063, 1066, 1067, 1070, 1072, 1073, 1074, 1075, 1076, 1077, 1078, 1079, 1082, 1086, 1094, 1096, 1101, 1103, 1104 and 1106 from the Floto Laboratory, Cambridge University, UK (https://www.flotolab.com/) M. abscessus clinical isolate library. Each individual isolate was assessed in duplicate.

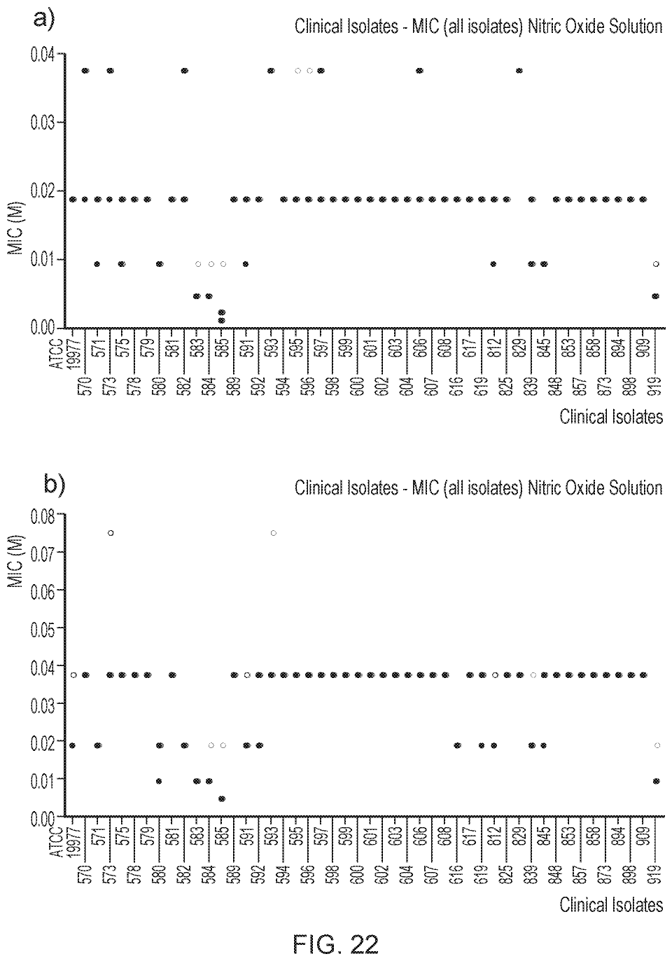

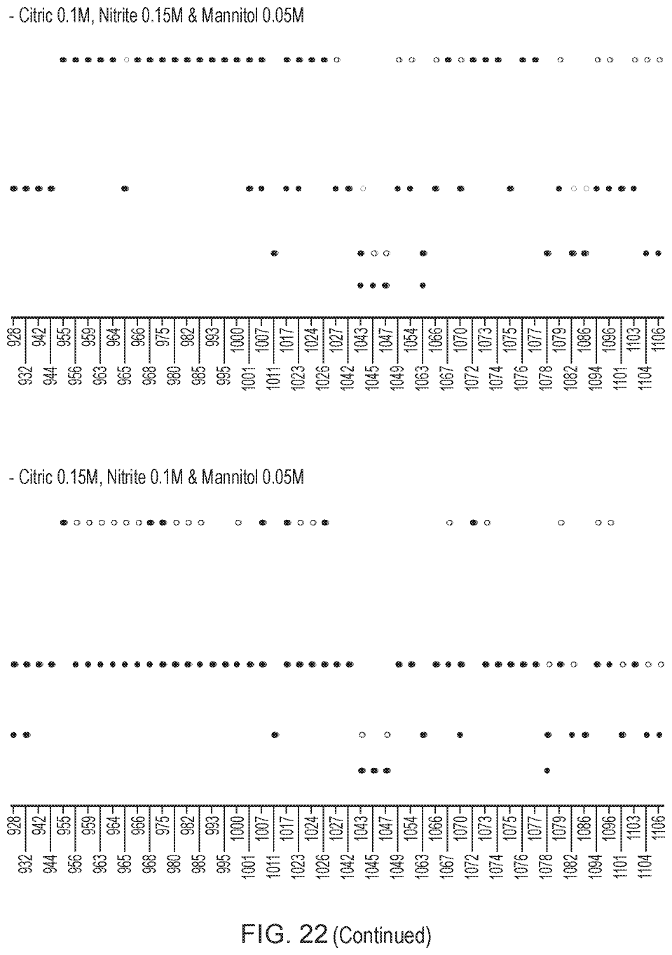

The results for the tests on the clinical isolates are shown in FIGS. 22 a) and b). The graphs show the MIC of nitric oxide against M. abscessus in duplicate with readings taken after three, four and five days of incubation of the isolates. The plates were also read at day 7 of incubation but there was no difference seen, compared to day 5. The laboratory strain ATCC 19977 lux was used as a control in both experiments and shows comparative results to the clinical isolates.

FIG. 22 shows that citric acid-nitrite-mannitol solutions have an effect across a broad range of clinical isolates. The minimum inhibition concentrations for a majority of clinical isolates were within 0.02 M for the 0.1 M citric acid, 0.15 M nitrite and 0.05 M mannitol solutions (FIG. 22a) and the minimum inhibition concentrations for a majority of clinical isolates were within 0.04 M for the 0.15 M citric acid, 0.1 M nitrite and 0.05 M mannitol solutions (FIG. 22b).

In both figures the MIC on certain samples differed on different days. Those are the samples with more than one dot shown above the identification code of the isolate sample. Generally speaking, in that situation the higher MIC was observed on later days of incubation than the lower MIC. Overall, the combination with the lower citric acid (0.1 M) and the higher sodium nitrite (0.15 M) (FIG. 22(a)) is more effective than the combination with the higher citric acid (0.15 M) and the lower sodium nitrite (0.1 M) (FIG. 22(b)).

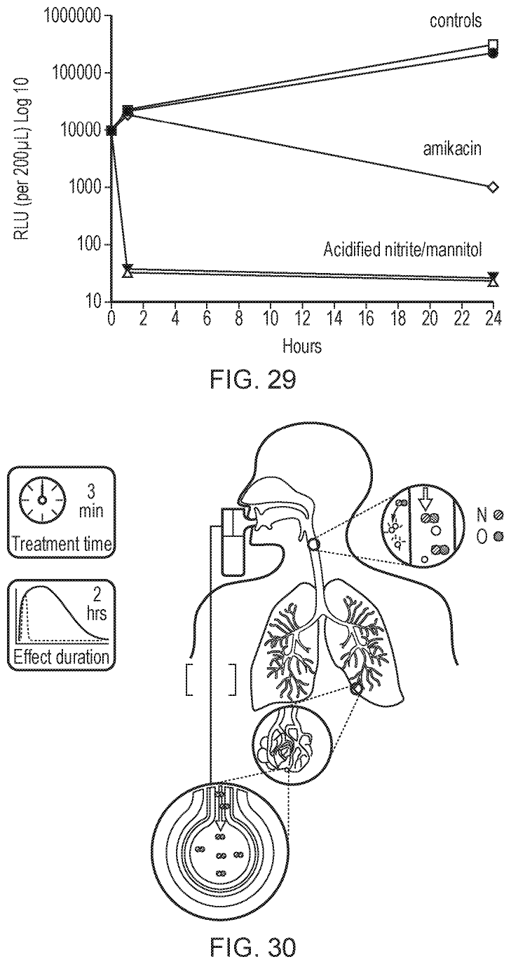

Additional data showing in vitro killing of M. abscessus by carboxylic acid-nitrite-polyol solutions is shown in FIG. 29. In this figure, the M. abscessus killing effectiveness of an aqueous formulation of sodium nitrite, citric acid buffered to pH 5.8 using sodium hydroxide solution, and mannitol is demonstrated in comparison with amikacin and negative controls over a 24 hour period under analogous conditions.

### Example 5

Antimicrobial Activity Against Pseudomonas aeruginosa for Carboxylic Acid-Nitrite Solutions with and without a Polyol

Equipment and Media

UKAS calibrated pipettes (100-1000 μL range)—Proline® Plus

UKAS calibrated multichannel pipettes (P300 and P20)—Gilson®, UK

Universal tubes—SLS, UK

Calibrated balance—HR-100A

Microbiological incubator—Heratherm™, ThermoFisher Scientific, UK

Tryptone Soya Agar (TSA)— Southern Group Laboratories, UK

Tryptone Soya Broth (TSB)—Acumedia®, SLS, UK

Malt Agar—Acumedia®, Acumedia®, SLS, UK

Brain Heart Infusion Broth (BHIB)—Acumedia®, SLS, UK

Sabouraud Dextrose Broth (SDB)—Acumedia®, SLS, UK

Dey-Engley Neutraliser (DE-N)— Acumedia®, SLS, UK

Citric Acid—Sigma, UK

Sodium Nitrite—Sigma, UK Mannitol—Sigma, UK

Sorbitol—Sigma, UK

Test Microorganisms

Pseudomonas aeruginosa NCTC 13618—Isolated from a cystic fibrosis patient

Formulations

| | | | |
|---|---|---|---|
| Formulation 1 | Liquid | Citric Acid pH 5.2 | sodium nitrite |
| Formulation 2 | Liquid | Citric Acid pH 6.0 | sodium nitrite |
| Formulation 3 | Liquid | Citric Acid pH 5.2 | sodium nitrite with |
| | | | mannitol |
| Formulation 4 | Liquid | Citric Acid pH 6.0 | sodium nitrite with |
| | | | mannitol |
| Formulation 5 | Liquid | Citric Acid pH 5.2 | sodium nitrite with |
| | | | sorbitol |
| Formulation 6 | Liquid | Citric Acid pH 6.0 | sodium nitrite with |
| | | | sorbitol |
| Positive control | Liquid | N/A | N/A |
| Negative control | Liquid | N/A | N/A |

Concentration 1-1 M Citric acid plus 1 M sodium nitrite (with or without 0.5 M polyol)

Concentration 2-0.5 M Citric acid plus 1 M sodium nitrite (with or without 0.5 M polyol)

Concentration 3-0.5 M Citric acid plus 0.5 M sodium nitrite (with or without 0.5 M polyol)

Dey-Engley Neutraliser Validation

Twenty-four-hour cultures of Pseudomonas aeruginosa were harvested from Tryptone Soya Agar (TSA) and used to prepare a 1×108±5×107 CFUmL⁻¹ suspension. This was further diluted in Brain Heart Infusion Broth (BHIB) to prepare a 1×105±5×104 CFUmL⁻¹ working suspension.

The starting inoculum was confirmed by serial dilution and spread plating. The neutraliser validation was performed using control (9 mL Phosphate Buffered Saline (PBS) and 1 mL inoculum), toxicity (9 mL Dey-Engley neutraliser (DE-N) and 1 mL inoculum), and neutraliser efficacy (8 mL neutraliser, 1 mL test agent and 1 mL inoculum) samples. Following a 5-minute treatment, 200 μL of suspension was removed from each tube, serially diluted and 100 μL was plated onto TSA. Agar plates were incubated at 37±2° C. for 18-24 hours.

Antimicrobial Efficacy Against Planktonic Organisms

Twenty-four-hour cultures of P. aeruginosa were harvested from TSA and used to prepare a 1×10⁸±5×107 CFUmL⁻¹ suspension. This was further diluted in BHIB to prepare a 1×10⁶±5×10⁴ CFUmL⁻¹ working suspension. Universal tubes were filled with 8 mL bacterial solution.

One ml of citric acid solution and 1 mL of sodium nitrite solution were added to each test agent to give the required concentration as described above. Solutions were incubated at 37±2° C. for 24 hours. Following the incubation period, 1 mL was removed from each tube and transferred to a tube containing 9 mL neutraliser. Viable organisms were quantified using serial dilutions and plate counting.

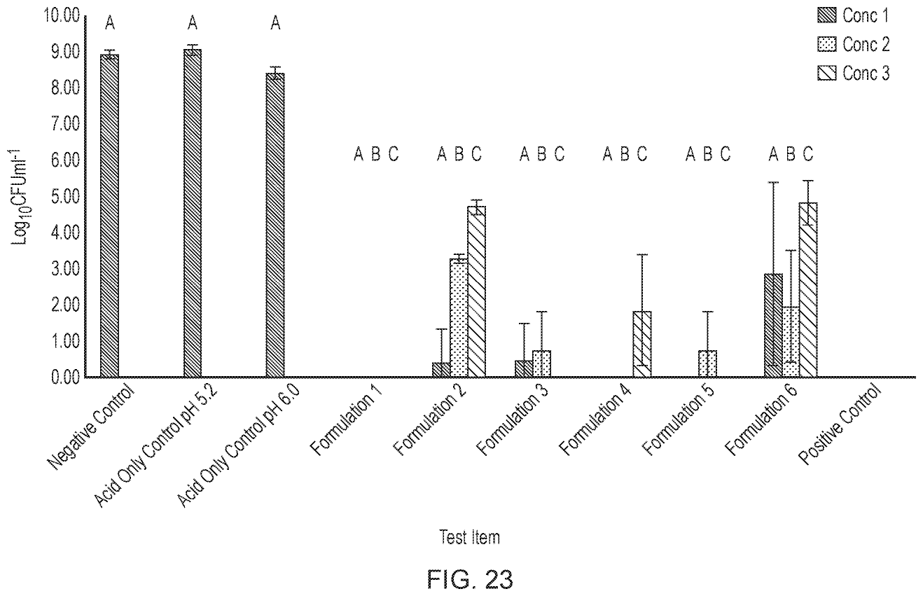

The results are shown in FIG. 23.

The data show the antimicrobial effectiveness against Pseudomonas of:
citric acid (1 M) mixed with nitrite (1 M) with and without polyol (0.5 M) (“Conc. 1”);
citric acid (0.5 M) mixed with nitrite (1 M) with and without polyol (0.5 M) (“Conc. 2”); and
citric acid (1 M) mixed with nitrite (0.5 M) with and without polyol (0.5 M) (“Conc. 3”).

The citric acid solution is at pH 5.2 (for Formulations 1, 3 and 5) and 6.0 (for Formulations 2, 4 and 6). Formulations 1 and 2 contain no polyol; Formulations 3 and 4 include mannitol; and Formulations 5 and 6 contain sorbitol.

Good efficacy is shown for all formulations at pH 5.2. At pH 6, the formulations comprising mannitol are marginally more effective.

### Example 6

The efficacy of formulations including nitrite salt, organic acid and polyol against M. tuberculosis HN 878 in THP-1 cells was evaluated.

Formulations

Formulations were prepared as set out in the following table. Where the preparation method is stated as “concentrate”, denoted by the suffix FC in the Sample Reference, this means that the formulation was initially made up as a concentrated pre-mix containing all three ingredients sodium nitrite (0.75M), polyol (0.25M) and acid (0.5M), and then diluted with distilled water to arrive at the desired concentration of each as stated in the table. Where the preparation method is stated as “dilute”, denoted by the suffix FD in the Sample Reference, this means that the formulation was initially made up as a pre-mix containing all three ingredients at the desired concentration initially, namely sodium nitrite (0.15M), polyol (0.05M) and acid (0.1M), and then diluted with distilled water to arrive at the desired concentration of each as stated in the table.

Within each formulation, a range of concentrations of the sodium nitrite was prepared by serial dilution, namely 16, 8, 4, 2, 1, 0.5, 0.25 and 0.125 μg/ml for the in vitro bacterial inhibition assays against M. tuberculosis HN878.

| | | | Test Mixture (final | | |
|---|---|---|---|---|---|
| | | | molarity post dilution) | | |
| | | | Sodium | | |
| | Sample Reference | Preparation Method | Nitrite | Polyol | Acid |
| Formulation 1 | 30RESP001FC | concentrate | sodium | mannitol | citric acid/ |
| | | | nitrite | 0.05 M | citrate 0.1 M |
| | | | 0.15 M | | |
| | 30RESP001FD | dilute | sodium | mannitol | citric acid/ |
| | | | nitrite | 0.05 M | citrate 0.1 M |
| | | | 0.15 M | | |
| Formulation 2 | 30RESP002FC | concentrate | sodium | lactitol | citric acid/ |
| | | | nitrite | 0.05 M | citrate 0.1 M |
| | | | 0.15 M | | |
| | 30RESP002FD | dilute | sodium | lactitol | citric acid/ |
| | | | nitrite | 0.05 M | citrate 0.1 M |
| | | | 0.15 M | | |
| Formulation 3 | 30RESP003FC | concentrate | sodium | mannitol | citric acid/ |
| | | | nitrite 0.1 M | 0.05 M | citrate 0.1 M |
| | 30RESP003FD | dilute | sodium | mannitol | citric acid/ |
| | | | nitrite 0.1 M | 0.05 M | citrate 0.1 M |
| Formulation 4 | 30RESP004FC | concentrate | sodium | mannitol | ascorbic acid/ |
| | | | nitrite 0.1 M | 0.05 M | ascorbate |
| | | | | | 0.1 M |
| | 30RESP004FD | dilute | sodium | mannitol | ascorbic acid/ |
| | | | nitrite 0.1 M | 0.05 M | ascorbate |
| | | | | | 0.1 M |

MIC macrophage testing was performed using a THP-1 macrophage (1) compound screening assay.

Macrophage Preparation and Culture: THP-1 cells were expanded for 2 weeks. Thereafter, THP-1 cells were suspended in complete DMEM media for macrophages at a concentration of 5×10⁵ cells/mL. The cells were seeded into 24 well tissue culture plates, 2 mL per well (1×10⁶ per well). One 24-well plate of cells allows for a range of 7 drug concentrations plus untreated controls to be tested in triplicate. In addition to the drug assay plates, one extra plate was seeded (or at least 3 additional wells) for determining bacterial uptake on the day of infection. The cells were incubated at 37° C. at 5% CO₂ in a humidified chamber. DMEM antiobiotic/antimycotic-free complete media were not changed during the 3 day assay.

Complete DMEM Media for Macrophages:

Dulbecco's Modification of Eagle's Medium (Cellgro 15-017-cv) supplemented with:

Heat-inactivated fetal calf serum (Atlas Biologicals, Fort Collins, Colo., F-0500-A) (10%)

L929-conditioned medium (10%)

L-glutamine (Sigma G-7513) (2 mM)

HEPES buffer (Sigma H-0887) (10 mM) Antibiotic/antimycotic (Sigma A-9909) (1X)

MEM non-essential amino acids (Sigma M-7145) (1X)

2-mercaptoethanol (Sigma M-6250) (50 nM)

L-929 Conditioned Media:

L-929 (CCL-1) cells from ATCC were seeded at 4.7×10⁵ cells in 55 mL of DMEM+10% fetal calf serum in a 75 cm² flask. Cells were allowed to grow for THP-1 cells 3 days. On day 3, the supernatant was collected and filtered through a 0.45-μm filter, aliquotted, and frozen at −20° C. The cell-free filtrate was used in the DMEM media for THP-1 infection.

Infection of THP-1 cells:

On day 0, the media was removed from the cells and replaced with 0.2 ml of antibiotic/antimycotic-free DMEM containing M. tuberculosis HN878 at a MOI of 1 macrophage to 10 bacteria ratio. The tissue culture plates were placed inside closed Ziploc baggies for transport back to the incubator. Once inside the incubator, the baggies were unzipped. The cells were incubated with the bacteria for 2 hours. After infection, the bacteria attached to the outside of the cells were removed, each well was washed once with phosphate buffered saline (PBS), and 2 mL of antibiotic/antimycotic-free complete DMEM media with various drug concentrations was added. To prepare the drug concentrations, serial 2 fold dilutions were performed by adding 10 ml of the previous suspension to 10 ml complete medium plus serum in the next tube. Tissue culture plates were returned to the incubator at 37° C.+5% CO₂ (drugs remained in wells for 3 days). Each drug concentration was tested in triplicate wells.

Plating of cell lysates and evaluation of cell viability for THP-1 cells was performed after 2 hours, 1, 2 and 5 days after infection. Tissue culture medium was removed from all wells, and cells were washed twice with 1 ml PBS. Next, 1 ml of sterile double distilled water+0.05% Tween-80 was added to each well; cells were left at room temperature for 5-10 min. Cell lysates were serially diluted 1:10 in sterile saline in 24-well tissue culture plates. Diluted cell lysates were plated onto 7H11/OADC agar through the 1/1,000 dilution step. (Each 24-well TC plate of cells requires four 24-well TC plates for making the serial dilutions, and 24 agar ‘quad’ plates). Plates were incubated at 32° C. for 30 days and colonies were enumerated to determine CFU/ml.

Results:

In vitro THP-1 HN878 Optical Density Results

Minimum Inhibitory Concentration (MIC), reported as the most dilute composition (i.e. the greatest dilution level of the particular formulation on the scale denoted as 16, 8, 4, 2, 1, 0.5, 0.25, 0.125 μg/ml) which inhibits the bacteria

| | MIC | MIC | MIC | MIC |
|---|---|---|---|---|
| | (μg/ml) | (μg/ml) | (μg/ml) | (μg/ml) |
| Compound | Day 0 | Day 1 | Day 2 | Day 5 |
| Formulation 1 | 16 | 16 | 16 | 16 |
| (30RESP001FC) | | | | |
| Concentrate | | | | |
| Formulation 1 | 16 | 16 | 16 | 16 |
| (30RESP001FD) | | | | |
| Dilute | | | | |
| Formulation 2 | 8 | 8 | 8 | 16 |
| (30RESP002FC) | | | | |
| Concentrate | | | | |
| Formulation 2 | 16 | 8 | 16 | 16 |
| (30RESP002FD) | | | | |
| Dilute | | | | |
| Formulation 3 | 8 | 8 | 8 | 8 |
| (30RESP003FC) | | | | |
| Concentrate | | | | |
| Formulation 3 | 8 | 16 | 16 | 16 |
| (30RESP003FD) | | | | |
| Dilute | | | | |
| Formulation 4 | 4 | 4 | 4 | 0.125 |
| (30RESP004FC) | | | | |
| Concentrate | | | | |
| Formulation 4 | 16 | 16 | 0.25 | 0.25 |
| (30RESP004FD) | | | | |
| Dilute | | | | |

The results are shown in FIGS. 24 to 27.

FIG. 24: the efficacy of 30RESP001FC and FD (concentrate and dilute) against M. tuberculosis HN878 was evaluated in THP-1 cells. The efficacy of formulations 30RESP001FC (concentrate) (A), and 30RESP001FD (dilute), (B), after 2 hours (Day 0), 1, 2 and 5 days after infection and treatment with 16 μg/ml (▴), 8 μg/ml ( [symbol] ), 4 μg/ml (⋄), 2 μg/ml (∘), 1 μg/ml (□), 0.5 μg/ml (♦), 0.25 μg/ml (▴), and 0.125 ομg/ml (▾) were evaluated for intracellular killing of M. tuberculosis HN878 ( [symbol] ) in THP-1 macrophages. In each of the plots in FIG. 24, the ▴ and [symbol] plot lines for treatment with 16 μg/ml and 8 μg/ml, respectively, can be distinguished from the ▴ and ▾ plot lines for treatment with 0.25 μg/ml and 0.125 μg/ml, respectively, because the treatments with 16 μg/ml and 8 μg/ml are more efficacious. In other words, the plot lines for treatment with 16 μg/ml and 8 μg/ml show significantly lower CFU values than treatment with 0.25 μg/ml and 0.125 μg/ml, particularly at day 5. Similar, the □ plot line for treatment with 1 μg/ml can easily be distinguished from the [symbol] plot line for no treatment because the treatment at 1 μg/ml is more efficacious. The [symbol] plot line for no treatment has CFU values that rise and remain above 1×10⁴ after day 1.

The 30RESP001FC and FD compositions referred to in the above MIC table and in FIG. 24 described as “16 μg/ml” comprise 0.15 M sodium nitrite, 0.05 M mannitol and 0.1 M citric acid/citrate (final molarity post-dilution), with the 8, 4, 2, 1, 0.5, 0.25 and 0.125 μg/ml compositions each respectively a 50% dilution (i.e. halving the concentration) of the previous composition in the said order 16 to 0.125 μg/ml.

THP-1 macrophages were infected with M. tuberculosis at a MOI of 1:10 and the numbers of intracellular bacteria were determined using the bacterial colony count method (CFU) immediately after 2 hours (Day 0), 1, 2 and 5 days after infection. Values shown are the mean±SD from one independent experiment. In particular, an increased efficacy relative to the untreated control was present in the treatment with 30RESP001FC and FD (concentrate and dilute) 16 μg/ml, and 8 μg/ml, against M. tuberculosis HN878 (*, p<0.05).

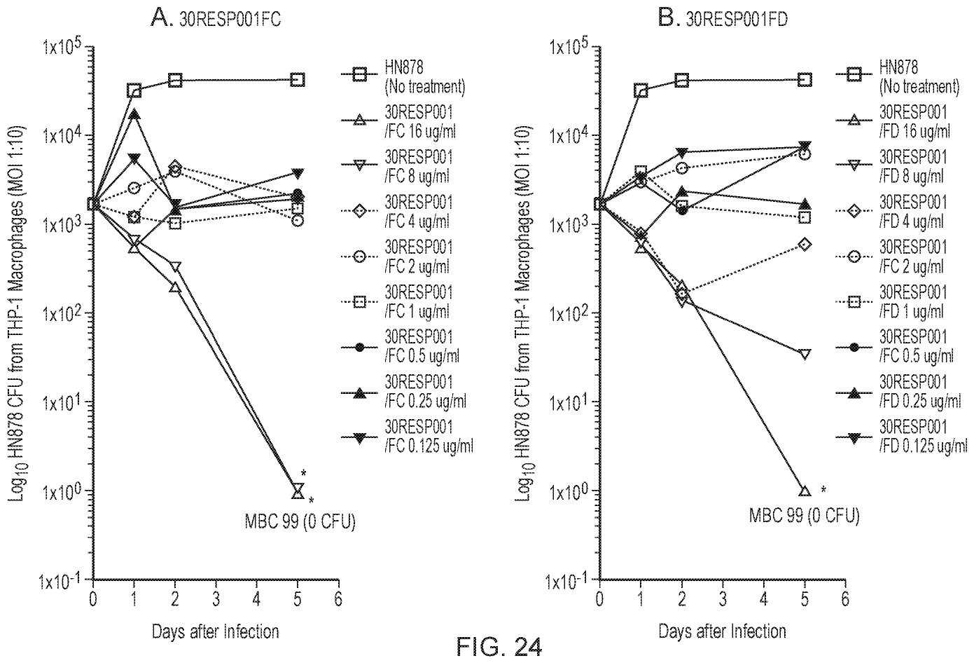

FIG. 25: the efficacy of 30RESP002FC and FD (concentrate and dilute) against M. tuberculosis HN878 was evaluated in THP-1 cells. The efficacy of formulations of 30RESP002FC (concentrate) (A), and 30RESP002FD (dilute), (B), after 2 hours, 1, 2 and 5 days after infection and treatment with 16 μg/ml (▴), 8 μg/ml ( [symbol] ) 4 μg/ml (⋄), 2 μg/ml (∘), 1 μg/ml (□), 0.5 μg/ml (♦), 0.25 μg/ml (▴) and 0.125 μg/ml (▾) were evaluated for intracellular killing of M. tuberculosis HN878 (o) in THP-1 macrophages. In each of the plots in FIG. 25, the ▴ and [symbol] plot lines for treatment with 16 μg/ml and 8 μg/ml, respectively, can be distinguished from the ▴ and ▾ plot lines for treatment with 0.25 μg/ml and 0.125 μg/ml, respectively, because the treatments with 16 μg/ml and 8 μg/ml are more efficacious. In other words, the plot lines for treatment with 16 μg/ml and 8 μg/ml show significantly lower CFU values than treatment with 0.25 μg/ml and 0.125 μg/ml, particularly at day 5. Similar, the □0 plot line for treatment with 1 μg/ml can easily be distinguished from the [symbol] plot line for no treatment because the treatment at 1 μg/ml is more efficacious. The [symbol] plot line for no treatment has CFU values that rise and remain above 1×10⁴ after day 1.

The 30RESP002FC and FD compositions referred to in the above MIC table and in FIG. 25 described as “16 μg/ml” comprise 0.15 M sodium nitrite, 0.05 M lactitol and 0.1 M citric acid/citrate (final molarity post-dilution), with the 8, 4, 2, 1, 0.5, 0.25 and 0.125 μg/ml compositions each respectively a 50% dilution (i.e. halving the concentration) of the previous composition in the said order 16 to 0.125 μg/ml.

THP-1 macrophages were infected with M. tuberculosis at a MOI of 1:10 and the numbers of intracellular bacteria were determined using the bacterial colony count method (CFU) immediately after 2 hours, 1, 2 and 5 days after infection. Values shown are the mean±SD from one independent experiment. Increased efficacy relative to the untreated control was present in the treatment with 30RESP002FC (concentrate) 16 μg/ml, and 30RESP002FD (dilute) 16 μg/ml and 8 μg/ml, against M. tuberculosis HN878 (*, p<0.05).

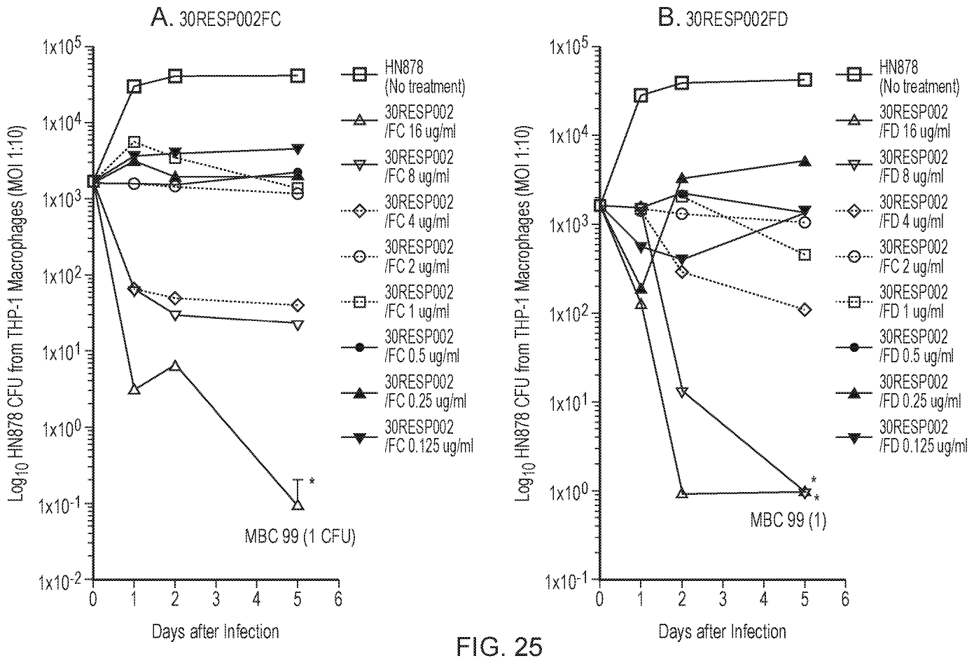

FIG. 26: the efficacy of 30RESP003FC and FD (concentrate and dilute) against M. tuberculosis HN878 was evaluated in THP-1 cells. The efficacy of 30RESP003FC (concentrate) (A), and 30RESP003FD (dilute), (B), after 2 hours (Day 0), 1, 2 and 5 days after infection and treatment with 16 μg/ml (▴), 8 μg/ml ( [symbol] ) 4 μg/ml (⋄), 2 μg/ml (∘), 1 μg/ml (□), 0.5 μg/ml (⋄), 0.25 μg/ml (▴), and 0.125 μg/ml (▾) were evaluated for intracellular killing of M. tuberculosis HN878 ( [symbol] ) in THP-1 macrophages. In each of the plots in FIG. 26, the ▴ and [symbol] plot lines for treatment with 16 μg/ml and 8 μg/ml, respectively, can be distinguished from the ▴ and ▾ plot lines for treatment with 0.25 μg/ml and 0.125 μg/ml, respectively, because the treatments with 16 μg/ml and 8 μg/ml are more efficacious. In other words, the plot lines for treatment with 16 μg/ml and 8 μg/ml show significantly lower CFU values than treatment with 0.25 μg/ml and 0.125 μg/ml, particularly at day 5. Similar, the □ plot line for treatment with 1 μg/ml can easily be distinguished from the [symbol] plot line for no treatment because the treatment at 1 μg/ml is more efficacious. The [symbol] plot line for no treatment has CFU values that rise and remain above 1×10⁴ after day 1.

The 30RESP003FC and FD compositions referred to in the above MIC table and in FIG. 26 described as “16 μg/ml” comprise 0.1 M sodium nitrite, 0.05 M mannitol and 0.1 M citric acid/citrate (final molarity post-dilution), with the 8, 4, 2, 1, 0.5, 0.25 and 0.125 μg/ml compositions each respectively a 50% dilution (i.e. halving the concentration) of the previous composition in the said order 16 to 0.125 μg/ml.

THP-1 macrophages were infected with M. tuberculosis at a MOI of 1:10 and the numbers of intracellular bacteria were determined using the bacterial colony count method (CFU) immediately after 2 hours, 1, 2 and 5 days after infection. Values shown are the mean±SD from one independent experiment. Increased efficacy relative to the untreated control was present in the treatment with 30RESP003FC (concentrate) 16 μg/ml and 8 μg/ml and 30RESP003FD 16 μg/ml, against M. tuberculosis HN878 (*, p<0.05).

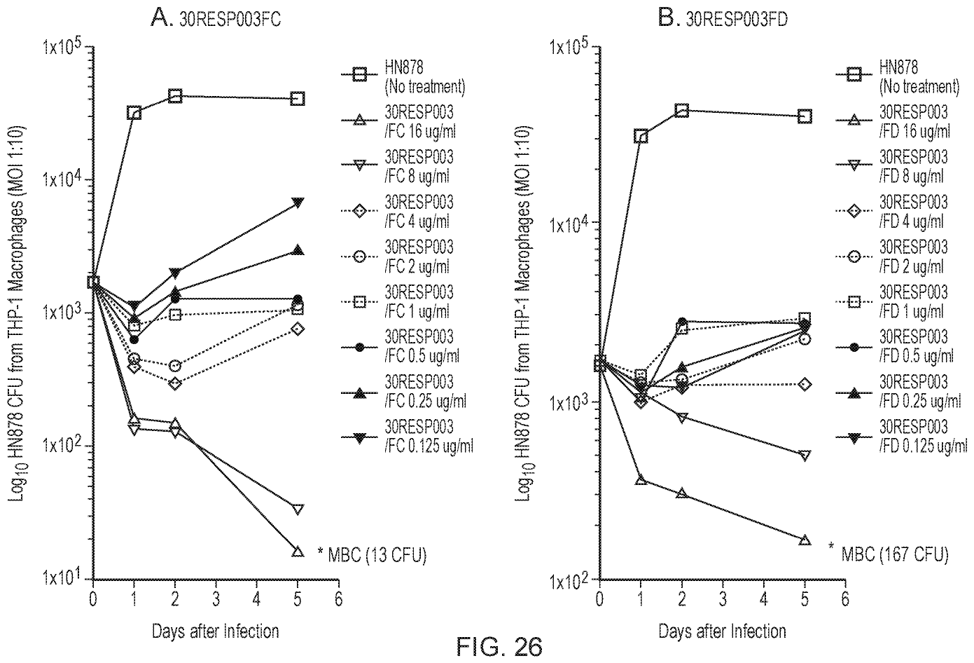

FIG. 27: the efficacy of 30RESP004FC and FD (concentrate and dilute) against M. tuberculosis HN878 was evaluated in THP-1 cells. The efficacy of formulations of 30RESP004FC (concentrate) (A), and 30RESP004FD (dilute), (B), after 2 hours (Day 0), 1, 2 and 5 days after infection and treatment with 16 μg/ml (▴), 8 μg/ml ( [symbol] ) 4 μg/ml (⋄), 2 μg/ml (∘), 1 μg/ml (□), 0.5 μg/ml (⋄), 0.25 μg/ml (▴) and 0.125 μg/ml (▾) were evaluated for intracellular killing of M. tuberculosis HN878 ( [symbol] ) in THP-1 macrophages. In each of the plots in FIG. 27, the ♦ and [symbol] plot lines for treatment with 16 μg/ml and 8 μg/ml, respectively, can be distinguished from the ▴ and ▾ plot lines for treatment with 0.25 μg/ml and 0.125 μg/ml, respectively, because the treatments with 16 μg/ml and 8 μg/ml are more efficacious. In other words, the plot lines for treatment with 16 μg/ml and 8 μg/ml show significantly lower CFU values than treatment with 0.25 μg/ml and 0.125 μg/ml, particularly at day 5. Similar, the □ plot line for treatment with 1 μg/ml can easily be distinguished from the [symbol] plot line for no treatment because the treatment at 1 μg/ml is more efficacious. The [symbol] plot line for no treatment has CFU values that rise and remain above 1×10⁴ after day 1.

The 30RESP004FC and FD compositions referred to in the above MIC table and in FIG. 27 described as “16 μg/ml” comprise 0.1 M sodium nitrite, 0.05 M mannitol and 0.1 M ascorbic acid/ascorbate (final molarity post-dilution), with the 8, 4, 2, 1, 0.5, 0.25 and 0.125 μg/ml compositions each respectively a 50% dilution (i.e. halving the concentration) of the previous composition in the said order 16 to 0.125 μg/ml.

THP-1 macrophages were infected with M. tuberculosis at a MOI of 1:10 and the numbers of intracellular bacteria were determined using the bacterial colony count method (CFU) immediately after 1, 2 and 5 days after infection. Values shown are the mean±SD from one independent experiment. Increased efficacy relative to the untreated control was present in the treatment with 30RESP004FC (concentrate) 16 μg/ml and 8 μg/ml, against M. tuberculosis HN878 (*, p<0.05).

It is concluded that the formulations show in vitro inhibition of M. tuberculosis HN878 at suitable dosages above MIC.

It will also be noted that the manner of making the Formulations has an effect on their in vitro antibacterial efficacy against M. tuberculosis HN878 in the tests of Example 6.

This is illustrated by comparing the efficacy of the 8 μg/ml concentration of Formulation 1 as between its FC and FD versions (FIG. 24A versus 24B). The efficacy of the FC version increases strongly for at least 5 days after incubation, whereas the efficacy of the FD version increases less strongly for the same time period. This is in contrast to the 16 μg/ml concentration, which shows very similar and good efficacy over the same period, as between the FC and FD versions.

Different behaviour is observed with Formulation 2 (FIG. 25A versus 25B). The efficacy of the 16 μg/ml concentration of the FD version increases more strongly than the FC version for the first 2 days after incubation and then does not change, although by 5 days after incubation the efficacy is good in the FD version and very good in the FC. In the case of the 8 μg/ml concentration, the efficacy of the FD version increases strongly to good efficacy for at least 5 days after incubation, whereas the efficacy of the FC version increases less strongly for the same time period.

It is thus shown that, at least at higher concentrations, the stage at which the water is added to arrive at the final inhibitory formulation, can materially affect the antibacterial action of the formulation both in terms of the initial antibacterial action and the extent of bacterial killing over 5 days. Generally speaking, although not universally, making the formulation initially as a concentrated pre-mix with the sodium nitrite, polyol and acid ingredients in their desired relative molar proportions but at a higher concentration than desired for use (e.g. at least 3 times, for example at least 5 times more concentrated than desired for use, for example from about 3 to about 80 times more concentrated than desired for use) and only then diluting the concentrate to obtain the formulation for use, leads to a better antibacterial action over the period of 0 to 5 days after incubation.

### Example 7

Cytotoxicity and Antiviral Activity of Carboxylic Acid-Nitrite-Polyol Solutions Against H1N1 Influenza a Virus

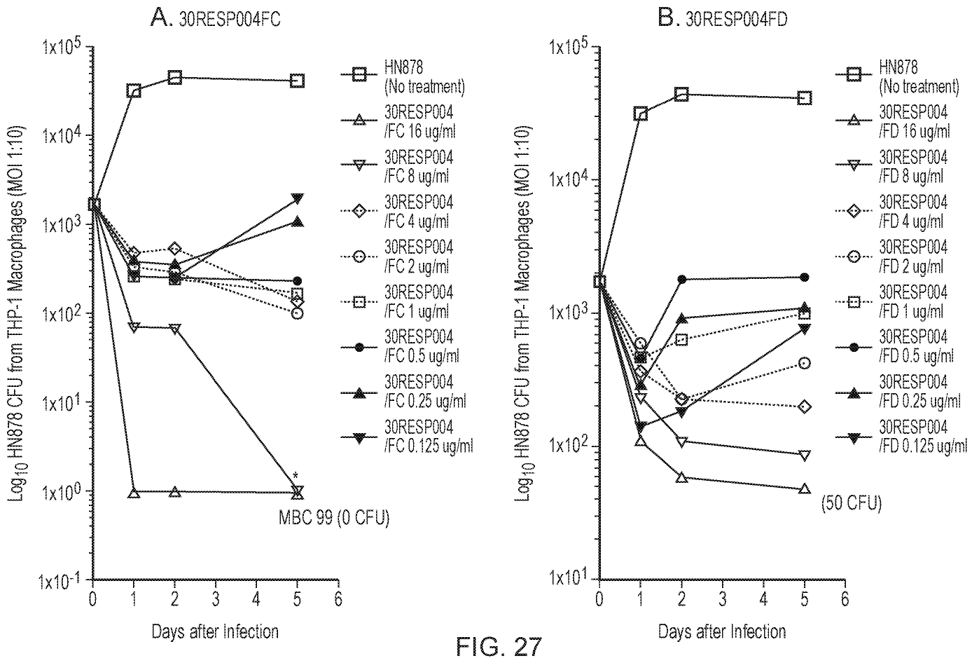

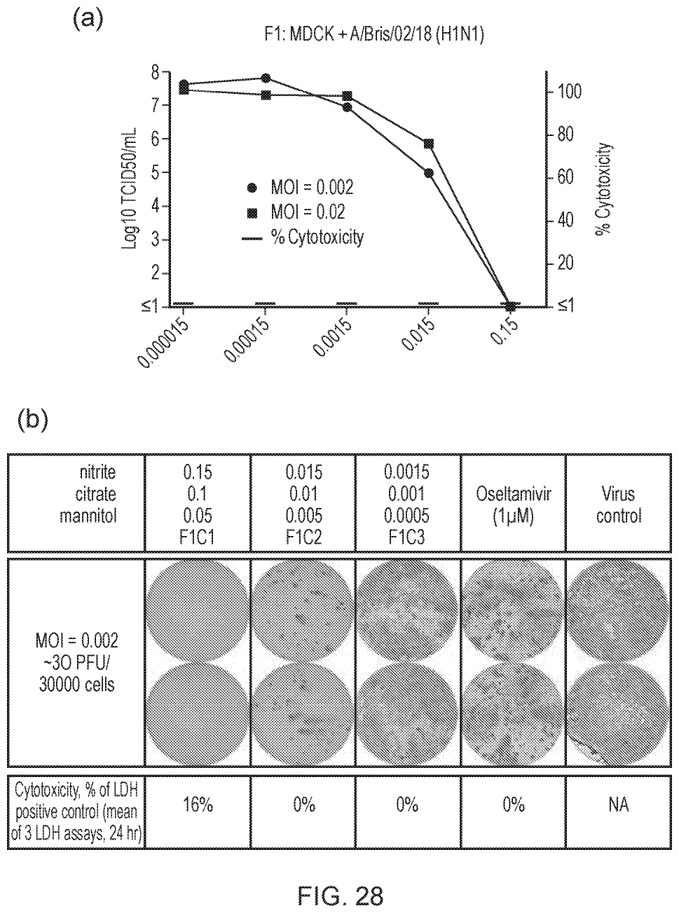

Test formulations designated F1C₁, F1C₂ and F1C₃ corresponding respectively to Formulation 30RESP001FC in Example 6, a 10-fold dilution thereof and a 100-fold dilution thereof, were used with oseltamivir solution (1 μM) and virus control to obtain comparative cytotoxicity and H1N1 Influenza A virus killing effect after 24 hours in MDCK cells. The cytotoxicity was assayed by LDH cytotoxicity assay analogously to Example 8. Antimicrobial activity against H1N1 Influenza A virus in MDCK cells was measured at MOI=0.002 (●) and MOI=0.02 (▪) at a range of dilutions (the horizontal axis is the nitrite molarity) with the cytotoxicity shown in grey, cytotoxicity scale on the right-hand side (cytoxicity at the measured nitrite concentrations up to and including 0.015M was ≤1% of LDH control). Plate photographs were obtained at MOI=0.002 and nitrite concentrations 0.15M, 0.015M and 0.0015M in comparison with oseltamivir (1 μM). The results are shown in FIG. 28. The order of the plates recited in the last-but-one sentence is the same as the order of the plates in the Figure going from left to right (there were two experiments, and the plates of each corresponding experiment are shown one above the other). The far right hand pair of plates, immediately to the right of the oseltamivir pair of plates, is the virus control. The cytotoxicity is shown below each pair of test plates, as the % of LDH control (mean of 3 LDH assays at 24 hours post-infection).

The results show that, at a suitable dose of the nitrite/citric acid/polyol formulation there is complete eradication of the virus, and it is clearly superior to oseltamivir. Similar antiviral activity of nitrite/citric acid/polyol formulations has been shown with rhinovirus and respiratory syncytial virus (RSV).

These results indicate that therapeutic and prophylactic treatments for respiratory viral infections in human and animal subjects are provided by nitrite/acid/polyol formulations in accordance with the present invention.

### Example 8

Cytotoxicity and Antiviral Activity of Carboxylic Acid-Nitrite-Polyol Solutions Against Coronavirus SARS-CoV-2

Materials

Test Formulation F1 (pH 5.8)

Six test concentrations of Formulation 1 (F1), being an aqueous solution of sodium nitrite, citric acid at pH 5.8 and mannitol (a polyol) were prepared by the method described below from stock solutions of 1.5M sodium nitrite, 0.91M citric acid/citrate buffer at pH 5.8, and 0.5M mannitol solution to give the following test compositions:

Formulation 1 (F1)

| | Concentration | Concentration of | |
|---|---|---|---|
| | of sodium | citric acid in test | Concentration |
| | nitrite in test | preparation (M) | of polyol in test |
| Test agent | preparation (M) | pH 5.8 | preparation (M) |
| F1 test cone 1 | 1.5 × 10⁻¹ M | 0.91 × 10⁻¹ M | 5.0 × 10⁻² M |
| (F1C1) | | | mannitol |
| F1 test cone 2 | 5.0 × 10⁻² M | 0.30 × 10⁻¹ M | 1.5 × 10⁻² M |
| (F1C2) | | | mannitol |
| F1 test cone 3 | 1.5 × 10⁻² M | 0.91 × 10⁻² M | 5.0 × 10⁻³ M |
| (F1C3) | | | mannitol |
| F1 test cone 4 | 1.5 × 10⁻³ M | 0.91 × 10⁻³ M | 5.0 × 10⁻⁴ M |
| (F1C4) | | | mannitol |
| F1 test cone 5 | 1.5 × 10⁻⁴ M | 0.91 × 10⁻⁴ M | 5.0 × 10⁻⁵ M |
| (F1C5) | | | mannitol |
| F1 test cone 6 | 1.5 × 10⁻¹ M | 0.91 × 10⁻¹ M | 2.5 × 10⁻² M |
| (F1C6) | | | mannitol |

Controls used with F1

A pH 5.8 control formulation was prepared from 0.1 M citric acid+assay buffer+cells.

A negative control was assay buffer+cells.

Positive controls were chloroquine+cells.

Test Formulation F2 (pH 5.4)

Six test concentrations of Formulation 2 (F2), being an aqueous solution of sodium nitrite, citric acid at pH 5.4 and mannitol (a polyol) were prepared by the method described below from stock solutions of 1.5M sodium nitrite, 0.91M citric acid/citrate buffer at pH 5.4, and 0.5M mannitol solution to give the following test compositions:

Formulation 2 (F2)

| | Concentration of | Concentration of | |
|---|---|---|---|
| | sodium | citric acid in test | Concentration of |
| | nitrite in test | preparation (M) | polyol in test |
| Test agent | preparation (M) | pH 5.4 | preparation (M) |
| F2 test cone 1 | 1.5 × 10⁻¹ M | 0.91 × 10⁻¹ M | 5.0 × 10⁻² M |
| (F2C1) | | | mannitol |
| F2 test cone 2 | 5.0 × 10⁻² M | 0.30 × 10⁻¹ M | 1.5 × 10⁻² M |
| (F2C2) | | | mannitol |
| F2 test cone 3 | 1.5 × 10⁻² M | 0.91 × 10⁻² M | 5.0 × 10⁻³ M |
| (F2C3) | | | mannitol |
| F2 test cone 4 | 1.5 × 10⁻³ M | 0.91 × 10⁻³ M | 5.0 × 10⁻⁴ M |
| (F2C4) | | | mannitol |
| F2 test cone 5 | 1.5 × 10⁻⁴ M | 0.91 × 10⁻⁴ M | 5.0 × 10⁻⁵ M |
| (F2C5) | | | mannitol |
| F2 test cone 6 | 1.5 × 10⁻¹ M | 0.91 × 10⁻¹ M | 2.5 × 10⁻² M |
| (F2C6) | | | mannitol |

Controls used with F2

A pH 5.4 control formulation was prepared from 0.1 M citric acid+assay buffer+cells.

A negative control was assay buffer+cells.

Positive controls were chloroquine+cells.

Chemical Reagents

Sodium nitrite:

Grade: Sodium nitrite extra pure Ph Eur, USP. Sodium nitrite CAS No. 7632-00-0, EC Number 231-555-9., extra pure Ph Eur, USP, from Sigma Aldrich, Product code 1.065441000.

Citric acid:

Grade: Citric acid anhydrous powder EMPROVE® ESSENTIAL Ph Eur, BP, JP, USP, E 330, FCC, from Sigma Aldrich, Product code 1.002425000.

D-Mannitol:

Grade: D-Mannitol that meets EP, FCC, USP testing specifications, from Sigma Aldrich, Product code M8429-100G.

Chloroquine phosphate:

Grade: Pharmaceutical Secondary Standard, from Sigma Aldrich, Product code PHR1258-1G.

Preparation of the Stock Solutions

To prepare the citric acid solution, one adds 90 ml of distilled water to 19.2 g citric acid, followed by 10 ml of 3M sodium hydroxide and then dilute with distilled water to adjust the pH (to 160 ml for pH 5.4 or 190 ml for pH 5.8). In an alternative method, one adds 20 ml of distilled water to 19.2 g citric acid, followed by 1.2 g solid sodium hydroxide and after that adjust the pH with 10M sodium hydroxide and distilled water to 100 ml. The solution is sterilised by syringe filtration using a 0.22ⱼun filter.

To prepare a 1.0 M sodium nitrite solution, 100 mL of distilled water was added to 6.9 g sodium nitrite. To prepare a 1.5 M sodium nitrite solution, 100 mL of distilled water was added to 10.35 g sodium nitrite.

When specified, 9.1 g of mannitol was added to give a concentration of 0.5 M. Sterilise solutions by syringe filtration using a 0.22 μm filter.

Preparation of the Formulations

The pH of the buffered citric acid solution is controlled to the desired value, prior to mixing with the nitrite and mannitol solutions. The pH stated for a formulation is the pH of the buffered citric acid solution as made up before mixing with the nitrite and mannitol solutions.

One suitable way to make up the formulations is as follows: Sodium nitrite (1.5 M) containing 0.5 M mannitol is added to a mixing vessel, immediately followed by the pH controlled citric acid solution in a 1:1 mix (nitrite+polyol: citric acid). The solutions are mixed by gentle inversion. Once mixed, the mixture is held for 5 minutes in a sealed container (e.g. a 50 ml falcon tube) at ambient temperature. The resulting solution containing 0.75 M nitrite, mannitol 0.25 M, and citric is then diluted 5-fold in assay buffer (1.2-fold concentrated) to give a final test concentration of nitrite 0.15 M, mannitol 0.05 M, and for example citric acid 0.1 M in the assay. Serial dilutions of the 1:1 mix (for example: a mix starting as nitrite 0.75M, mannitol 0.25M, citric acid 0.5M) are made with distilled water and/or the assay buffer medium. All formulation concentrations can be stored at ambient temperature. Solutions are made fresh for each run.

Additional Controls

As additional controls were used S-nitroso-N-acetylpenicillamine (SNAP) at a range of concentrations known to be suitable for its purpose and denoted SNAP50, SNAP100, SNAP200, SNAP300 and SNAP400. SNAP is a known NO donor serving as a positive NO donating control in these tests to provide verification that NO is not cytotoxic in vitro. To control out any potential effect on the assay of the N-acetylpencillamine (NAP) portion of the SNAP molecule, corresponding concentrations of NAP were used as an NO blank control and denoted NAP50, NAP100, NAP200, NAP300 and NAP400.

Virus

SARS-CoV-2 clinical isolate.

Cell Line

Vero E6.

Assays

LDH assay (cytotoxicity):

CyQUANT™ LDH Cytotoxicity Assay Kit, Invitrogen; Cat No. C₂₀₃₀₀ and C₂₀₃₀₁. Tissue culture infectious dose (TCID50) was determined (virus titration) using cytopathic effect (CPE) scoring as readout.

The cytotoxicity of the nitrite formulations (all concentrations), pH 5.8 or pH 5.4 citrate control, negative controls and positive controls (chloroquine, as described by Keyaerts, E, Biochem Biophys Res Commun, 323, 264-268 (2004), the contents of which are incorporated herein by reference) was tested at 2 hr and 24 hr post nitrite/control addition on the Vero E6 cells. LDH release was measured as the readout at the 2 hr and 24 her time points. Each compound/formulation was tested three times per run.

Sars-Cov-2 Inhibition:

At time 0 hr, Vero E6 cells were infected with virus in presence of the formulation or controls and incubated for 1 hour. After this incubation period the inoculum was removed and the cells were washed. Fresh formulation or controls were then added to the washed cells. At 24 hours post infection, Vero E6 cell supernatants were harvested and titrated, and the virus titration was incubated for 6 days prior to readout to determine any virus yield reduction. Separate tests were performed at four MOIs including 3.0 and 0.3, although only those two MOIs were titrated. The readout was by crystal violet (cell monolayer) staining, followed by CPE scoring.

Results

The results are shown in FIGS. 32 to 34.

FIG. 32 shows the results of the LDH cytotoxicity assay (combined graph from Runs 1 and 2, using respectively Test Formulations 1 and 2). The data is expressed as mean+standard deviation (SD) of two experiments. SD shown as the grey error bars. The maximum LDH activity (cells+lysis buffer) was set at 100% and all sample results are relative to this value. The LDH positive control was the positive control from the kit. The black bars (2 hour incubation) are the left-hand bar of each pair of bars in each case, and the red bars (24 hour incubation) are the right-hand bar of each pair of bars in each case.

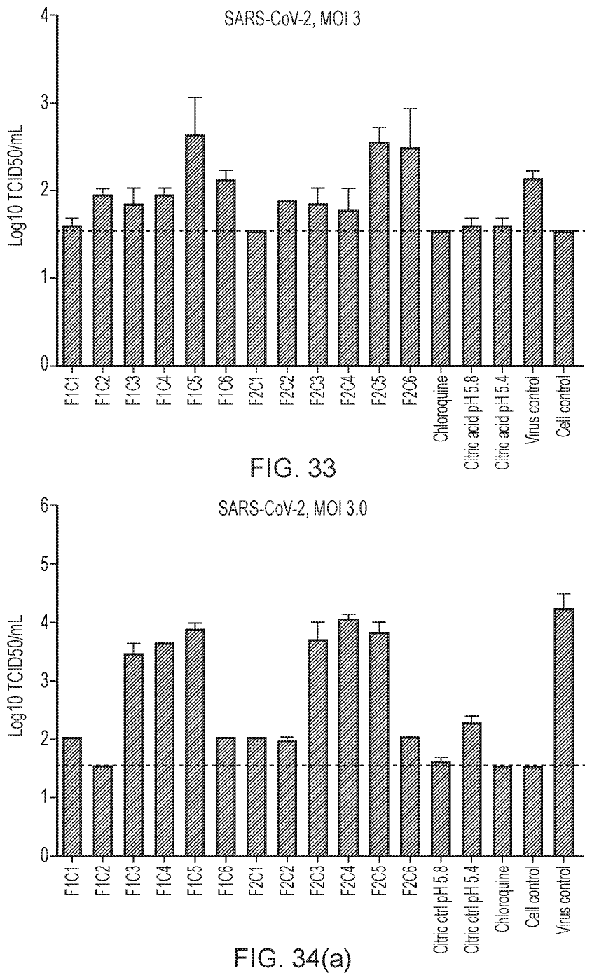

FIG. 33 shows the results of the antiviral testing against SARS-CoV-2 of Run 1 at MOI 3.0. In Run 1, one virus yield reduction assay was performed using SARS-CoV-2 at four multiplicities of infection (MOIs), confirmed using back titration of the inoculum virus. For cells inoculated with an MOI of 3, 2.1 log 10 TCID50/m1 was found in the virus control well after titration. Reduction of SARS-CoV-2 yield might be observed for some of the conditions tested. After 24 hours of incubation, hardly any virus was detected in the lowest three MOIs (i.e. 0.3, 0.03 and 0.003). Possibly, 24 hours of replication on Vero E6 cells is not sufficient for obtaining high levels of progeny virus. The data is expressed as mean+standard deviation (SD) of two titrations. SD shown as the error bars. The horizontal dotted line level with the chloroquine and cell control log 10 TCID50/ml values is the limit of detection (LOD) of the assay.

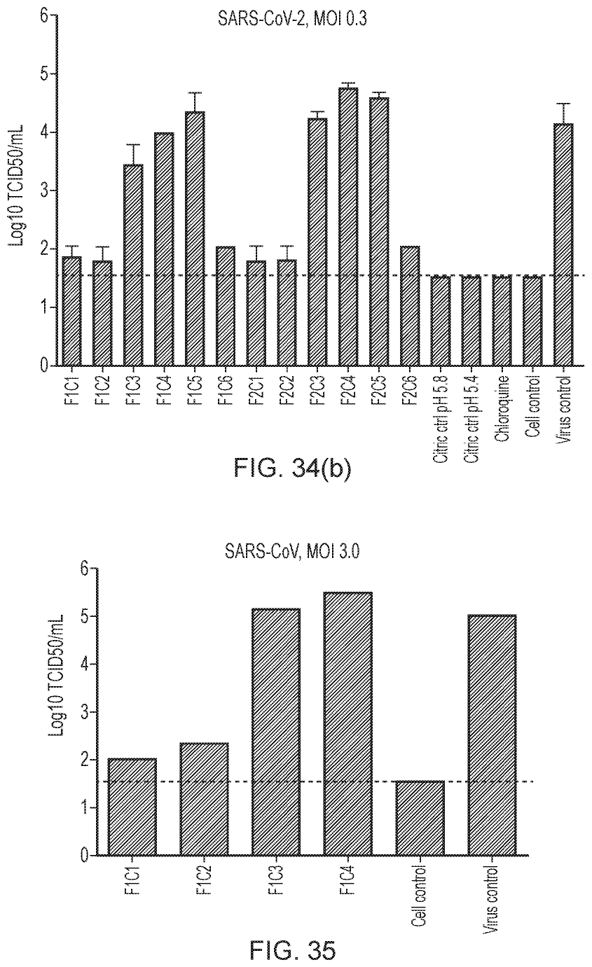

FIG. 34 shows the results of the antiviral testing against SARS-CoV-2 of Run 2 (a) at MOI 3.0 and (b) at MOI 0.3. The methodology corresponds to the parts of Run 1 at those MOIs, with the exception that the formulations are the Run 2 formulations (Test Formulation 2 at its various concentrations) and incubation was performed for 48 hours rather than 24 hours, in order to increase the level of progeny virus. The data is expressed as mean+standard deviation (SD) of two titrations. SD shown as the error bars. The horizontal dotted line level with the chloroquine and cell control log 10 TCID50/ml values is the limit of detection (LOD) of the assay.

Discussion

The NO generating aqueous formulations are not cytotoxic on the LDH assay (FIG. 32). Particularly at the higher concentrations of nitrite, acid and polyol the in vitro antiviral action against SARS-Cov-2 is impressive and comparable with chloroquine (FIGS. 33 and 345).

The NO generating aqueous formulations are effective at a surprisingly high pH. pH 5.4 and 5.8 were tested, but lower pH down to 5.2 or even below would also be expected to have efficacy.

Furthermore, the data reveal that organic carboxylic acids (such as citric acid buffered to pH 5.4 or 5.8), in the absence of an NO generating formulation, have a surprising low cytotoxicity and high in vitro antiviral action against SARS-CoV-2 (FIGS. 32 to 34; “citric acid pH 5.8” and “citric acid pH 5.4” bars). The relatively high pH for a carboxylic acid formulation makes such formulations attractive as intrapulmonary active agents as they will be expected to be non-toxic to airway and lung tissue surfaces. Since SARS-Cov-2 belongs to the same coronavirus family as SARS-Cov and there are similarities between the viruses, it is reasonable to predict also that such organic carboxylic acids will show corresponding efficacy against SARS-CoV virus, the coronavirus that is responsible for severe acute respiratory syndrome (SARS), of which there was a well documented outbreak in 2002 and 2003.

### Example 9

Antiviral Activity of Carboxylic Acid-Nitrite-Polyol Solutions Against Coronavirus SARS-CoV

To investigate analogies between the antiviral activity provided by the present invention against SARS-CoV-2 and that provided by the present invention against SARS-CoV, the following experiment was performed.

Formulations F1C₁, F1C₂, F1C₃ and F1C₄ were tested for antiviral activity against SARS-CoV at MOI 3.0. The methodology was analogous to the antiviral testing described in Example 8. Prior to cell monolayer staining with crystal violet, 2 plates were microscopically checked and scored for cytopathic effect (CPE). A CPE, in the form of cell debris on top of an underlying monolayer, was found to be present in these plates.

The results of the two plates, that were microscopically checked, is shown in FIG. 35. Data are a single titration per condition. For the remaining plates, no CPE could be scored after crystal violet staining, due to a too dense cell monolayer. The horizontal dotted line level with the cell control log 10 TCID50/ml value is the limit of detection (LOD) of the assay.

As shown in FIG. 35, at least the formulations F1C₁ and F1C₂ provided good in vitro antiviral activity against SARS-CoV.

### Example 10

Inhaler for Human Use

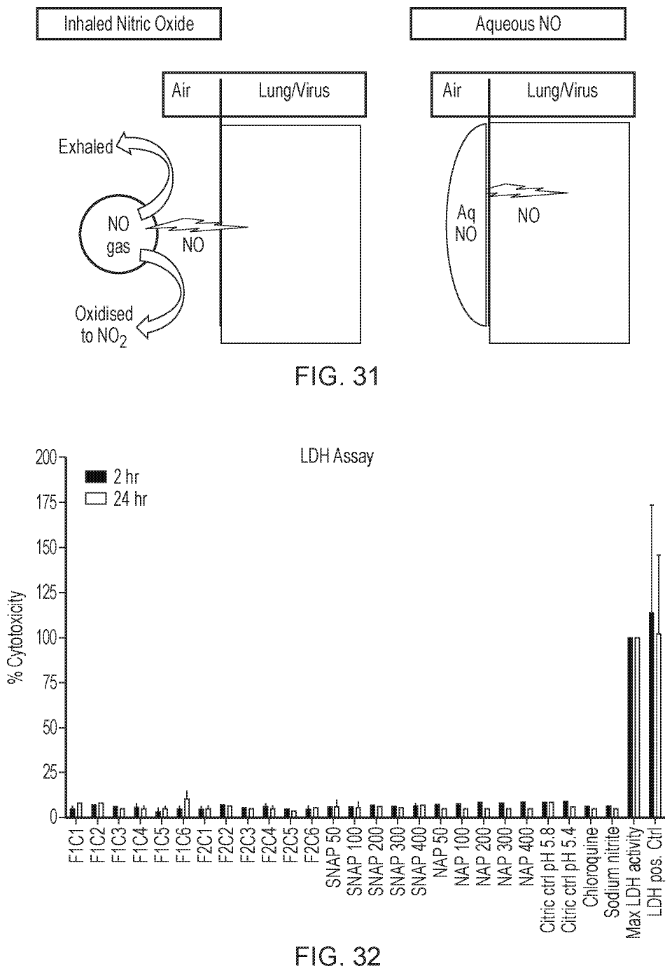

An embodiment of an inhaler for human use employing a liquid composition according to the present invention is shown schematically in FIGS. 30 and 31.

The inhaler is suitably powered by a compressed gas and configured to deliver one dose of entrained droplets of the nitrite/acid/polyol formulation from a reservoir in the inhaler in response to one manual actuation of the inhaler, in generally conventional manner. The subject typically inhales at the same time as actuating the inhaler, as is conventionally done by asthma sufferers when using their inhalers. As shown in FIG. 30, a treatment time of about 3 minutes per dose should be suitable, giving a duration of effect of up to about 2 hours with a suitable dose of the active composition.

The airborne droplets travel into the subject's infected lungs, where they contact the infected (e.g. virus-infected) membranes of the lungs. FIG. 31 shows on the right hand side the effect of the present invention in depositing multiple droplets of the aqueous nitric oxide (NO) generating composition (“Aqueous NO”) on the lining of the lungs. FIG. 31 shows on the left hand side the corresponding effect if—instead of the aqueous nitric oxide (NO) generating composition—gaseous nitric oxide is inhaled by the subject (“Inhaled Nitric Oxide”).

As shown, the efficacy is likely to be much reduced if Inhaled Nitric Oxide would be used. Not only is a proportion of the inhaled nitric oxide breathed out by the subject before it can pass into the bloodstream through the membrane lining of the lungs, but another proportion of the inhaled nitric oxide is oxidised to toxic nitrogen dioxide (NO₂) by oxygen in the inhaled air. The nitrogen dioxide has an adverse effect on the subject's lungs, in addition to depleting the availability of gaseous nitric oxide for treating the subject.

As a result, a more efficient and effective delivery of nitric oxide to the lungs of the patient and into the patient's bloodstream via the lungs is achieved by using a nitrite/acid/polyol formulation in accordance with the present invention.

### CONCLUSION

The foregoing broadly describes the present invention without limitation. Variations and modifications as will be readily apparent to those skilled in the art are intended to be included within the scope of the appended claims. To the extent that the laws of any particular jurisdiction in or for which a patent is granted to this invention provide for enforcement of the patent against unauthorised use of technology which is equivalent to the appended claims, the proprietor intends that the patent covers such equivalent technology.

Equivalents of the protective scope of the appended claims are also covered by the claims to the extent permitted by applicable law. For example, generally speaking the order of mixing the components or portions of components of the NOx generating reaction described herein is not critical, provided that the NOx generating reaction is not prematurely initiated. Any order of mixing of essential and non-essential components of any combination, kit or composition of the present invention is intended to be covered. If one or more component is used in liquid form, e.g. as solutions, then the effect of the admixture of that component or those components on the concentration of solutes (including but not limited to that component or those components) in the reaction mixture or any component part of the reaction mixture is likely to be different, compared with the case where that one or more component would be used in solid form or in a liquid form at a different volume or concentration. The use of all equivalent concentrations and/or physical forms (solid, liquid, solutions) of components to form the combinations, kits and compositions of the present invention, and all equivalent steps and orders of steps to prepare the said combinations, kits and compositions, even if not described or specifically claimed herein, is within the scope of the present claims to the extent permitted by applicable law.

1. A method for generating nitric oxide, optionally other oxides of nitrogen and/or optionally precursors thereof for the treatment of wounds, skin lesions and/or burns, comprising reacting one or more nitrite salt with a proton source comprising one or more acid selected from organic carboxylic acids and organic non-carboxylic reducing acids under reaction conditions suitable to generate nitric oxide, optionally other oxides of nitrogen and/or optionally precursors thereof, wherein the reaction is performed in the process of one or more organic polyol, wherein one or more of the nitrite salt, proton source or organic polyol is present in solution in an aqueous carrier, for example an aqueous liquid or gel;
characterised by: the one or more organic polyol does not comprise propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol.

2. The method according to claim 1 further characterised by one or more of the following:
(a) the one or more organic polyol is present in a reaction output enhancing amount;
(b) the proton source is not solely a hydrogel comprising pendant carboxylic acid groups covalently bonded to a three-dimensional polymeric matrix;
(c) the one or more organic polyol is not solely glycerol;
(d) the one or more organic polyol is not solely glycerol when one or more viscosity increasing agent is used;
(e) the one or more organic polyol is not solely glycerol when one or more plasticizer is used;
(f) the one or more organic polyol is not solely polyvinyl alcohol;
(g) the one or more organic polyol is not solely polyvinyl alcohol when one or more viscosity increasing agent is used; and
(h) any one or more of (b) to (g) above, wherein the words “is not solely” are replaced by “does not comprise”.

3. A combination for generating nitric oxide, optionally other oxides of nitrogen and/or optionally precursors thereof by reaction of one or more nitrite salt with a proton source, the combination comprising:
one or more nitrite salt;
(ii) a proton source comprising one or more acid selected from organic carboxylic acids and organic non-carboxylic reducing acids; and
(iii) one or more organic polyol;
wherein one or more of the nitrite salt, proton source or organic polyol is present in solution in an aqueous carrier, for example an aqueous liquid or gel;
characterised by the one or more organic polyol does not comprise propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol.

4. The combination according to claim 3 further characterised by one or more of the following:
(a) the one or more organic polyol is present in a reaction output enhancing amount;
(b) the proton source is not solely a hydrogel comprising pendant carboxylic acid groups covalently bonded to a three-dimensional polymeric matrix;
(c) the one or more organic polyol is not solely glycerol;
(d) the one or more organic polyol is not solely glycerol when one or more viscosity increasing agent is used;
(e) the one or more organic polyol is not solely glycerol when one or more plasticizer is used;
(f) the one or more organic polyol is not solely polyvinyl alcohol;
(g) the one or more organic polyol is not solely polyvinyl alcohol when one or more viscosity increasing agent is used;
(h) any one or more of (b) to (g) above, wherein the words “is not solely” are replaced by “does not comprise”.

5. A kit for generating nitric oxide, optionally other oxides of nitrogen and/or optionally precursors thereof by reaction of one or more nitrite salt with a proton source, the kit comprising:
one or more nitrite salt;
(ii) a proton source comprising one or more acid selected from organic carboxylic acids and organic non-carboxylic reducing acids; and
(iii) one or more organic polyol;
wherein one or more of the nitrite salt, proton source or organic polyol is present in solution in an aqueous carrier, for example an aqueous liquid or gel;
characterised by the one or more organic polyol does not comprise propylene glycol, polyethylene glycol, glycerin monostearate (glyceryl stearate), trihydroxyethylamine, D-pantothenyl alcohol, panthenol, panthenol in combination with inositol, butanediol, butenediol, butynediol, pentanediol, hexanediol, octanediol, neopentyl glycol, 2-methyl-1,3-propanediol, ethylene glycol, diethylene glycol, triethylene glycol, tetraethylene glycol, dipropylene glycol, dibutylene glycol, butane-1,2,3-triol, butane-1,2,4-triol, hexane-1,2,6-triol, hexylene glycol, caprylyl glycol, glycols other than those listed here, hydroquinone, butylated hydroquinone, 1-thioglycerol, erythorbate, ethylhexylglycerin, any combination thereof, or any combination of any of the above with glycerol and/or polyvinyl alcohol.

6. The kit according to claim 5 further characterised by one or more of the following:
(a) the one or more organic polyol is present in a reaction output enhancing amount;
(b) the proton source is not solely a hydrogel comprising pendant carboxylic acid groups covalently bonded to a three-dimensional polymeric matrix;
(c) the one or more organic polyol is not solely glycerol;
(d) the one or more organic polyol is not solely glycerol when one or more viscosity increasing agent is used;
(e) the one or more organic polyol is not solely glycerol when one or more plasticizer is used;
(f) the one or more organic polyol is not solely polyvinyl alcohol;
(g) the one or more organic polyol is not solely polyvinyl alcohol when one or more viscosity increasing agent is used; and
(h) any one or more of (b) to (g) above, wherein the words “is not solely” are replaced by “does not comprise”.

7.-64. (canceled)
